(19) United States
(12) Patent Application Publication
(10) Pub. No.: US 2007/0293515 A1
(43) Pub. Date: Dec. 20, 2007

(54) 1,3-Disubstituted Heteroaryl Nmda/Nr2b Antagonists

(76) Inventors: Mark E. Layton, Harleysville, PA (US); Kevin J. Rodzinak, Schwenksville, PA (US); Michael J. Kelly III, Wayne, PA (US); Philip E. Sanderson, Valley Forge, PA (US)

Correspondence Address: MERCK AND CO., INC, P O BOX 2000, RAHWAY, NJ, 07065-0907, US

(21) Appl. No.: 11/659,454

(22) PCT Filed: Jul. 29, 2005
(86) PCT No.: PCT/US05/27160

§ 371 Date: Feb. 2, 2007

Related U.S. Application Data
(60) Provisional application No. 60/598,521, filed on Aug. 3, 2004.

(51) Int. Cl. A61K 31/519; A61P 25/04; C07D 487/04

(52) U.S. Cl. 514/262.1; 544/262

(57) ABSTRACT

Compounds represented by Formula I: 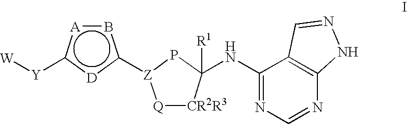
(wherein A, B, D, P, Q, R¹, R², R³, W and Y are described herein) or pharmaceutically acceptable salts thereof, are effective as NMDA/NR2B antagonists useful for treating neurological conditions such as, for example, pain, Parkinson's disease, Alzheimer's disease, epilepsy, depression, anxiety, ischemic brain injury including stroke, and other conditions.

### FIELD OF THE INVENTION

This invention relates to 1,3 disubstituted heteroaryl compounds. In particular, this invention relates to 1,3 disubstituted heteroaryl compounds that are NMDA/NR2B antagonists useful for the treatment of neurological conditions such as pain, Parkinson's disease, Alzheimer's disease, epilepsy, depression, anxiety, ischemic brain injury including stroke, and other conditions.

### BACKGROUND OF THE INVENTION

Ions such as glutamate play a key role in processes related to chronic pain and pain-associated neurotoxicity—primarily by acting through N-methyl-D-aspartate (“NMDA”) receptors. Thus, inhibition of such action—by employing ion channel antagonists, particularly NMDA antagonists—can be beneficial in the treatment and control of Parkinon's disease and pain.

NMDA receptors are heteromeric assemblies of subunits, of which two major subunit families designated NR1 and NR2 have been cloned. Without being bound by theory, it is generally believed that the various functional NMDA receptors in the mammalian central nervous system (“CNS”) are only formed by combinations of NR1 and NR2 subunits, which respectively express glycine and glutamate recognition sites. The NR2 subunit family is in turn divided into four individual subunit types: NR2A, NR2B, NR2C, and NR2D. T. Ishii, et al., J. Biol. Chem., 268:2836-2843 (1993), and D. J. Laurie et al., Mol. Brain. Res., 51:23-32 (1997) describe how the various resulting combinations produce a variety of NMDA receptors differing in physiological and pharmacological properties such as ion gating properties, magnesium sensitivity, pharmacological profile, as well as in anatomical distribution.

For example, while NR1 is found throughout the brain, NR2 subunits are differentially distributed. In particular, it is believed that the distribution map for NR2B lowers the probability of side effects while treating Parkinson's disease or pain. Thus, it would be desirable to provide novel NM-DA antagonists that target the NR2B receptor.

### SUMMARY OF THE INVENTION

The present invention relates to 1,3-disubstituted-heteroaryl compounds represented by Formula I: 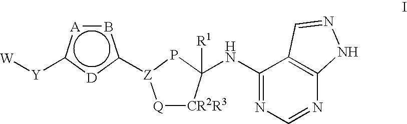
(wherein A, B, D, P, Q, R¹, R², R³, W and Y are described herein) or pharmaceutically acceptable salts thereof. This invention further provides methods to treat and prevent neurological conditions, including pain, Parkinson's disease, Alzheimer's disease, epilepsy, depression, anxiety, ischemic brain injury including stroke, and other conditions, utilizing the present compounds and compositions.

### DETAILED DESCRIPTION OF THE INVENTION

The compounds of this invention are represented by Formula I: 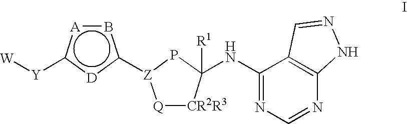
and pharmaceutically acceptable salts and individual enantiomers and stereoisomers thereof, wherein:
W is aryl or heteroaryl, unsubstituted or substituted with 1-5 substituents independently selected from halogen, C₁₋₆ alkyl optionally substituted with one or more substituents selected from halogen, hydroxyl and C₁₋₄ alkoxy, C₁₋₄ alkoxy, C₃₋₆ cycloalkyl, cyano and N(R⁴)₂;
Y is absent or is selected from C₁₋₃ alkyl optionally substituted with one or more substituents selected from halogen, hydroxyl and C₁₋₄ alkoxy, cyclopropyl and C(O);
Z is N and P and Q are C(R⁴)₂, or Z is CR⁵ and P and Q are C(R⁵)₂;
A, B and D are each independently selected from O, CR⁶, S and NR⁶;
R¹, R⁴ and R⁶ are each independently selected from hydrogen or C₁₋₄ alkyl optionally substituted with one or more of halogen and hydroxyl; and
R², R³, and R⁵ are each independently selected from hydrogen, halogen, hydroxyl, C₁₋₄ alkyl optionally substituted with one or more substituents selected from halogen and hydroxyl, C₁₋₄ alkoxy, cyano and N(R⁴)₂.

A further embodiment of the present invention includes compounds represented by Formula Ia: 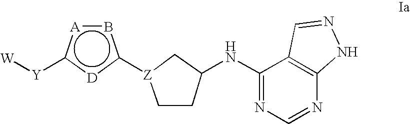
and pharmaceutically acceptable salts and individual enantiomers and stereoisomers thereof, wherein:
W is aryl or heteroaryl, unsubstituted or substituted with 1-5 substituents independently selected from halogen, C₁₋₆ alkyl optionally substituted with one or more substituents selected from halogen, hydroxyl and C₁₋₄ alkoxy, C₁₋₄ alkoxy, C₃₋₆ cycloalkyl, cyano and N(R⁴)₂;
Y is absent or is selected from C₁₋₃ alkyl optionally substituted with one or more substituents selected from halogen, hydroxyl and C₁₋₄ alkoxy, cyclopropyl and C(O);
Z is N or Z is CR⁵;
A, B and D are each independently selected from O, CR⁶, S and NR⁶;
R⁴ and R⁶ are each independently selected from hydrogen or C₁₋₄ alkyl, where said alkyl is unsubstituted or is substituted with halogen, hydroxyl or C₁₋₄ alkoxy; and
R⁵ is independently selected from hydrogen, halogen, hydroxyl, C₁₋₄ alkyl optionally substituted with one or more substituents selected from halogen and hydroxyl, C₁₋₄ alkoxy, cyano and N(R⁴)₂.

Another embodiment of this invention includes compounds represented by Formula Ib: 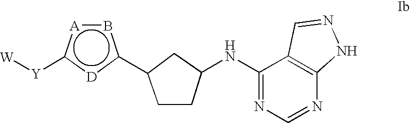
and pharmaceutically acceptable salts and individual enantiomers and stereoisomers thereof, wherein:
W is aryl or heteroaryl, unsubstituted or substituted with 1-5 substituents independently selected from halogen, C₁₋₆ alkyl optionally substituted with one or more of halogen, hydroxyl and C₁₋₄ alkoxy, C₁₋₄ alkoxy, C₃₋₆ cycloalkyl, cyano, and N(R⁴)₂;
Y is absent or is selected from C₁₋₃ alkyl unsubstituted or substituted with one or more substituents selected from halogen, hydroxyl and C₁₋₄ alkoxy, cyclopropyl and C(O);
A, B and D are each independently selected from O, CR⁶, S, and NR⁶;
R⁴ and R⁶ are each independently hydrogen, C₁₋₄ alkyl unsubstituted or substituted with one or more substituents selected from halogen and hydroxyl.

In yet another embodiment, the compounds of this invention are represented by Formula Ic: 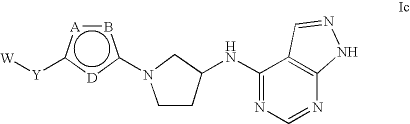
and pharmaceutically acceptable salts and individual enantiomers and stereoisomers thereof, wherein:
W is aryl or heteroaryl, unsubstituted or substituted with 1-5 substituents independently selected from, halogen, C₁₋₆ alkyl unsubstituted or substituted with one or more of halogen, hydroxyl or C₁₋₄ alkoxy, C₁₋₄ alkoxy, C₃₋₆ cycloalkyl, cyano and N(R⁴)₂;
Y is absent or is selected from C₁₋₃ alkyl unsubstituted or substituted with one or more substituent selected from halogen, hydroxyl and C₁₋₄ alkoxy, cyclopropyl and C(O);
A, B and D are each independently selected from O, CR⁶, S, and NR⁶; and
R⁴ and R⁶ are each independently hydrogen, C₁₋₄ alkyl unsubstituted or substituted with one or more substituent selected from halogen and hydroxyl.

As used herein, “alkyl” is intended to mean linear, branched and cyclic structures having no double or triple bonds. Thus C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl and hexyl. Similarly, C₁₋₄alkyl is defined to identify the group as having 1, 2, 3 or 4 carbons in a linear or branched arrangement, such that C₁₋₄alkyl specifically includes methyl, ethyl, n-propyl, iso-propyl, n-butyl, iso-butyl and tert-butyl. “Cycloalkyl” is an alkyl, part or all of which forms a ring of three or more atoms.

As used herein, “aryl” is intended to mean any stable monocyclic or bicyclic carbon ring of up to 7 members in each ring, wherein at least one ring is aromatic. Examples of such aryl elements include phenyl, napthyl and tolyl.

The term “heteroaryl”, as used herein except where noted, represents a stable 5- to 7-membered monocyclic- or stable 9- to 10-membered fused bicyclic heterocyclic ring system which contains an aromatic ring, any ring of which may be saturated, such as piperidinyl, partially saturated, or unsaturated, such as pyridinyl, and which consists of carbon atoms and from one to four heteroatoms selected from the group consisting of N, O and S, and wherein the nitrogen and sulfur heteroatoms may optionally be oxidized, and the nitrogen heteroatom may optionally be quaternized, and including any bicyclic group in which any of the above-defined heterocyclic rings is fused to a benzene ring. The heterocyclic ring may be attached at any heteroatom or carbon atom which results in the creation of a stable structure. Examples of such heteroaryl groups include, but are not limited to, pyridine, pyrimidine, pyrazine, thiophene, oxazole, thiazole, 1,2,4-oxadiazole, 1,3,4-oxadiazole, 1,2,4-thiadiazole, 1,3,4-thiadiazole, and 1,2,4-triazole.

In the structures depicted throughout this application a hydrogen atom on an unsubstituted nitrogen atom may be either expressly shown or implicit.

The same convention also applies to all generic structures and structures depicting individual species. Of course, nitrogen atoms may also be substituted with atoms and/or moieties other than hydrogen, as set forth elsewhere in this application.

As appreciated by those of skill in the art, halo or halogen as used herein are intended to include chloro, fluoro, bromo and iodo. Similarly, C₁₋₆, as in C₁₋₆alkyl is defined to identify the group as having 1, 2, 3, 4, 5 or 6 carbons in a linear or branched arrangement, such that C₁₋₆alkyl specifically includes methyl, ethyl, n-propyl, iso-propyl, n-butyl, iso-butyl, tert-butyl, pentyl, and hexyl. Likewise, C₀, as in C₀alkyl is defined to identify the presence of a direct covalent bond.

The term “substituted” is intended to include substitution at any or all positions. Thus, substitution can be made at any of the groups. For example, substituted aryl(C₁₋₆)alkyl includes substitution on the aryl group as well as substitution on the alkyl group.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. Formula I (including Formula Ia, Ib and Ic) is shown without a definitive stereochemistry at certain positions. The present invention includes all stereoisomers of Formula I and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

The independent syntheses of these diastereomers or their chromatographic separations may be achieved as known in the art by appropriate modification of the methodology disclosed herein. Their absolute stereochemistry may be determined by the x-ray crystallography of crystalline products or crystalline intermediates which are derivatized, if necessary, with a reagent containing an asymmetric center of known absolute configuration.

If desired, racemic mixtures of the compounds may be separated so that the individual enantiomers are isolated. The separation can be carried out by methods well known in the art, such as the coupling of a racemic mixture of compounds to an enantiomerically pure compound to form a diastereomeric mixture, followed by separation of the individual diastereomers by standard methods, such as fractional crystallization or chromatography. The coupling reaction is often the formation of salts using an enantiomerically pure acid or base. The diasteromeric derivatives may then be converted to the pure enantiomers by cleavage of the added chiral residue. The racemic mixture of the compounds can also be separated directly by chromatographic methods utilizing chiral stationary phases, which methods are well known in the art.

Alternatively, any enantiomer of a compound may be obtained by stereoselective synthesis using optically pure starting materials or reagents of known configuration by methods well known in the art.

As used herein, “pharmaceutically acceptable salts” refer to derivatives wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

When the compound of the present invention is basic, salts may be prepared from pharmaceutically acceptable non-toxic acids, including inorganic and organic acids. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. Particularly preferred are citric, hydrobromic, hydrochloric, maleic, phosphoric, sulfuric, fumaric, and tartaric acids. It will be understood that, as used herein, references to the compounds of Formula I are meant to also include the pharmaceutically acceptable salts.

The term “pharmaceutically acceptable salts” refers to salts prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like. Particularly preferred are citric, hydrobromic, hydrochloric, maleic, phosphoric, sulfuric, and tartaric acids.

The subject treated in the present methods is generally a mammal, preferably a human being, male or female, in whom antagonism of NMDA NR2B receptor activity is desired. The term “therapeutically effective amount” means the amount of the subject compound that will elicit the biological or medical response of a tissue, system, animal or human that is being sought by the researcher, veterinarian, medical doctor or other clinician. As used herein, the term “treatment” refers both to the treatment and to the prevention or prophylactic therapy of the mentioned conditions, particularly in a patient who is predisposed to such disease or disorder.

The term “composition” as used herein is intended to encompass a product comprising the specified ingredients in the specified amounts, as well as any product which results, directly or indirectly, from combination of the specified ingredients in the specified amounts. Such term in relation to pharmaceutical composition, is intended to encompass a product comprising the active ingredient(s), and the inert ingredient(s) that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing a compound of the present invention and a pharmaceutically acceptable carrier. By “pharmaceutically acceptable” it is meant the carrier, diluent or excipient must be compatible with the other ingredients of the formulation and not deleterious to the recipient thereof.

The terms “administration of” and or “administering a” compound should be understood to mean providing a compound of the invention or a prodrug of a compound of the invention to the individual in need of treatment.\

The pharmaceutical compositions of the present invention comprise a compound represented by Formula I (and/or pharmaceutically acceptable salt(s) thereof) as an active ingredient, a pharmaceutically acceptable carrier, and, optionally, other therapeutic ingredients or adjuvants. The instant compositions include those suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

The present invention is further directed to a method for the manufacture of a medicament for the antagonism of NMDA/NR2B receptor activity in humans and animals comprising combining a compound of the present invention with a pharmaceutical carrier or diluent.

In practice, the compounds represented by Formula I, or pharmaceutically acceptable salts thereof, of this invention can be combined as the active ingredient in intimate admixture with a pharmaceutical carrier according to conventional pharmaceutical compounding techniques. The carrier may take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compound represented by Formula I, and/or pharmaceutically acceptable salt(s) thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

Thus, the pharmaceutical compositions of this invention may include a pharmaceutically acceptable carrier and a compound or a pharmaceutically acceptable salt of Formula I. The compounds of Formula I, or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.5 mg to about 5 g of the active ingredient and each cachet or capsule preferably containing from about 0.5 mg to about 5 g of the active ingredient.

The pharmaceutical compositions of the present invention comprise a compound represented by Formula I (or pharmaceutically acceptable salts thereof) as an active ingredient, a pharmaceutically acceptable carrier, and optionally one or more additional therapeutic agents or adjuvants. The instant compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, utilizing a compound represented by Formula I of this invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in moulds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound described by Formula I, and/or pharmaceutically acceptable salts thereof, may also be prepared in powder or liquid concentrate form.

The utility of the compounds in accordance with the present invention as antagonists of NMDA/NR2B receptor activity may be demonstrated by methodology known in the art. Inhibition of the binding to NMDA receptors and functional antagonism of calcium efflux through NMDA channels were determined as follows:

Cell-Based Functional Assay to Determine IC₅₀ of NR2B Antagonists

The ability of selected compounds to inhibit NR1a/NR2B NMDA receptor, as measured by NR1a/NR2B receptor-mediated Ca²⁺ influx, was assessed by the following calcium flux assay procedure:

NR1a/NR2B receptor transfected L(tk−) cells were plated in 96-well format at 3×10⁴ cells per well and grown for one to two days in normal growth medium (Dulbeccos MEM with Na pyruvate, 4500 mg glucose, pen/strep, glutamine, 10% FCS and 0.5 mg/mL geneticin). NR1a/NR2B-expression in these cells was induced by the addition of 4-20 nM dexamethasone in the presence of 500 μM ketamine for 16-24 hours. Solutions of NR2B antagonists were prepared in DMSO and serially diluted with DMSO to yield 10 solutions differing by 3-fold in concentration. A 96-well drug plate was prepared by diluting the DMSO solution 250-fold into assay buffer (Hanks Balanced Salt Solution (HBSS) Mg²⁺ free (Gibco #14175-079) containing 20 mM HEPES, 2 mM CaCl₂, 0.1% BSA and 250 □M Probenecid (Sigma # P-8761)). After induction, the cells were washed twice (Labsystem cell washer, 3 fold dilutions leaving 100 □L) with assay buffer and loaded with 4 □M of the calcium fluorescence indicator fluo-3 AM (Molecular Probes # P-1241) in assay buffer containing Pluronic F-127 (Molecular Probes # P-3000) and 100M ketamine at 37° C. for one hour. The cells were then washed eight times with assay buffer leaving 100 μL of buffer in each well. Fluorescence intensity was immediately measured in a FLIPR (Fluorometric Imaging Plate Reader, Molecular Devices) using an excitation of 488 nm and emission at 530 nm. Five seconds after starting the recording of fluorescence intensity, 50 □L of agonist solution (40 □M glutamate/glycine, the final concentration 10 □M) was added and after one minute, when fluorescence signal was stable, 50 □L of NR2B antagonists and control solutions from the drug plate were added and the fluorescence intensity recorded for another 30 minutes. The IC₅₀ values were determined by a non-linear least squares fitting of the endpoint fluorescence values to Equation #1 below.

$$Endpoint Florescent = \frac{( Y max - Y min )}{1 + {( [ Drug ] / {IC}_{50} )}^{nH}} + Y min \qquad Equation \#1$$

where, Ymin is average endpoint fluorescence of the control wells containing 1 □M of AMD-2 and Ymax is the average endpoint fluorescence of wells containing 0.1% DMSO in assay buffer.

### Binding Assay to Determine K₁ NR2B Antagonists

The radioligand binding assay was performed at room temperature in 96-well microtiter plates with a final assay volume of 1.0 mL in 20 mM Hepes buffer (pH 7.4) containing 150 mM NaCl. Solutions of NR2B antagonists were prepared in DMSO and serially diluted with DMSO to yield 20 μL of each of 10 solutions differing by 3-fold in concentration. Non-specific binding (NSB) was assessed using AMD-1 (10 μM final concentration), and total binding (TB) was measured by addition of DMSO (2% final concentration). Membranes expressing NR^{1a}/NR2B receptors (40 □M final concentration) and tritiated AMD-2 (1 nM final concentration) were added to all wells of the microtiter plate. After 3 hours of incubation at room temperature, samples are filtered through Packard GF/B filters (presoaked in 0.05% PEI, polyethyleninine Sigma P-3143) and washed 10 times with 1 mL of cold 20 mM Hepes buffer per wash. After vacuum drying of the filter plates, 40 μL of Packard Microscint-20 was added and bound radioactivity determined in a Packard TopCount. The apparent dissociation constant (K₁), the maximum percentage inhibition (% Iₘₐₓ), the minimum percentage inhibition (% Iₘᵢₙ) and the hill slope (nH) were determined by a non-linear least squares fitting the bound radioactivity (CPM bound) to Equation #2 below.

$$\begin{matrix} CPM Bound = \frac{( SB ) ( \% I max - \% I min ) / 100}{( 1 + {( [ Drug ] / ( {K}_{I} ( 1 + [ AMD - 2 ] / {K}_{D} ) ) )}^{nH} )} + NSB + ( SB ) ( 100 - \% {I}_{max} ) / 100 & Equation \#2\end{matrix}$$

where, K_{D} is the apparent dissociation constant for the radioligand for the receptor as determined by a hot saturation experiment and SB is the specifically bound radioactivity determined from the difference of TB and NSB control wells.
Synthesis of AMD-1 and AMD-2 may be accomplished according to the following reaction schemes:

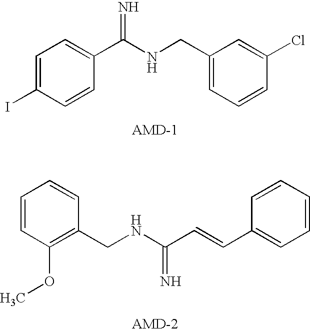

The precursor E for the synthesis of radiolabelled AMD-1 can be synthesized in accordance with the following procedure:

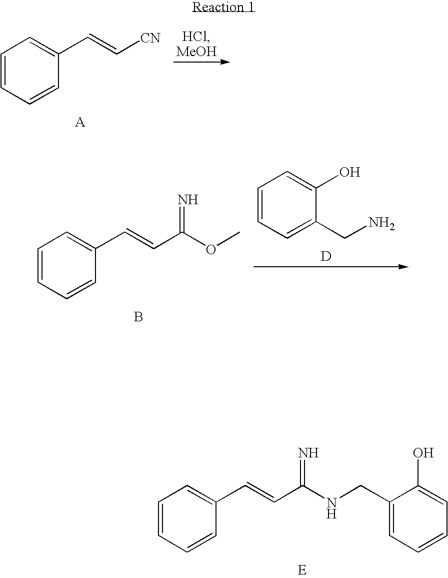

In accordance with the procedures of Reaction 1, hydrogen chloride is bubbled through a solution of cinnamonitrile A in methanol at room temperature. The volatiles are removed under reduced pressure and the resulting residue is triturated with ether and filtered to yield the intermediate imidate B. Imidate B is dissolved in methanol at ambient temperature, treated with amine D (commercially available from Acros Chemicals) at ambient temperature and stirred under argon. The volatiles are removed under reduced pressure and the residue purified by preparative HPLC or trituration with ether to afford amidine E.
TITRATED AMD-2 CAN BE SYNTHESIZED ACCORDING TO THE FOLLOWING PROCEDURE:

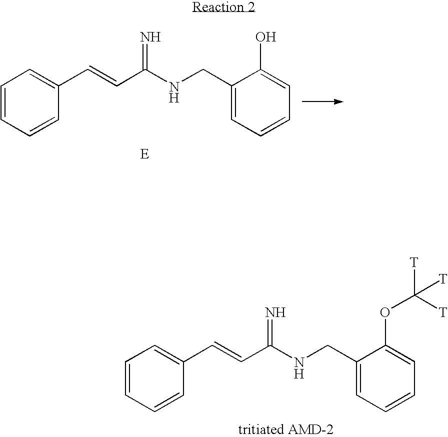

Tritiated AMD-2 was prepared by the following procedure, illustrated above in Reaction 2: The precursor E (2 mg, 0.008 mmol) dissolved in dimethylformamide (0.6 mL) and potassium carbonate (1.2 mg) for 1 h. High specific activity tritiated methyl iodide (50 mCi, 0.0006 mmol, in toluene 1 mL, commercially available from American Radiolabeled Chemicals) was added at room temperature and stirred for 2 hours. The reaction mixture was filtered using a Whatman PTFE 0.45 cm syringeless filter device to remove any insoluble potassium carbonate, washed with Abs. ethanol (2 mL, commercially available from Pharmco), and the combined filtrates were concentrated to dryness at room temperature using a rotary evaporator; this also removed any unreacted tritiated methyl iodide. The residue was purified by HPLC chromatography on a Phenomenx Luna C8 semi-prep column (Luna 5 micro C8(2), 250×10.0 mm) using a gradient system of 20/80 acetonitrile/water with 0.1% trifluoroacetic acid to 100% acetonitrile with 0.1% trifluoroacetic acid in 20 min. Total activity of the product was 8 mCi. Further purification was effected by absorption onto a Waters C-18 Sep-pak column (Waters Sep-Pak PLUS C18) and elution with water followed by absolute ethanol. The product was diluted with absolute ethanol (1 mL) before submission for final analysis.

AMD-1 can be synthesized according to the general procedure described by C. F. Claiborne et al (Bioorganic & Med. Chem. Letters 13, 697-700 (2003).

Unlabelled AMD-2 is prepared as follows:

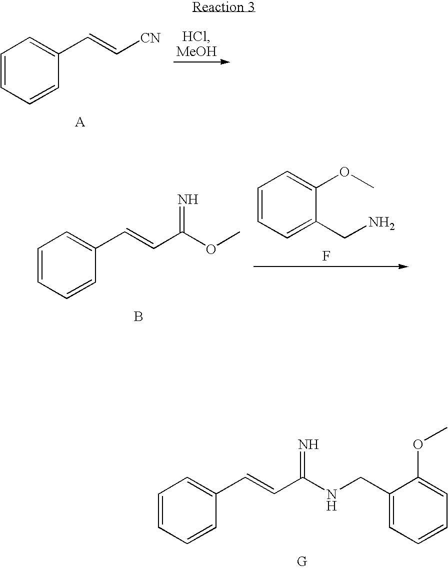

In accordance with Reaction 3, hydrogen chloride is bubbled through a solution of cinnamonitrile A in methanol at room temperature. The volatiles are removed under reduced pressure and the resulting residue is triturated with ether and filtered to yield the intermediate imidate B. Imidate B is dissolved in methanol at ambient temperature, treated with amine F at ambient temperature and stirred under argon. The volatiles are removed under reduced pressure and the residue purified by preparative HPLC or trituration with ether to afford amidine G.

The compounds of this invention exhibit IC₅₀ and K₁ values of less than 50 μM in the functional and binding assays, respectively. It is advantageous that the IC₅₀ and K₁ values be less than 5 μM in the functional and binding assays, respectively. It is more advantageous that the IC₅₀ and K, values be less than 1 μM in the functional and binding assays, respectively. It is still more advantageous that the IC₅₀ and K₁ values be less than 0.1 μM in the functional and binding assays, respectively.

The present compounds are NMDA NR2B receptor antagonists, and as such are useful for the treatment and prophylaxis of diseases and disorders mediated through the NR2B receptor. Such diseases and disorders include, but are not limited to, Parkinson's disease, neuropathic pain (such as postherpetic neuralgia, nerve injury, the “dynias”, e.g., vulvodynia, phantom limb pain, root avulsions, painful diabetic neuropathy, painful traumatic mononeuropathy, painful polyneuropathy), central pain syndromes (potentially caused by virtually any lesion at any level of the nervous system), and postsurgical pain syndromes (eg, postmastectomy syndrome, postthoracotomy syndrome, stump pain)), bone and joint pain (osteoarthritis), repetitive motion pain, dental pain, cancer pain, myofascial pain (muscular injury, fibromyalgia), perioperative pain (general surgery, gynecological), chronic pain, dysmennorhea, as well as pain associated with angina, and inflammatory pain of varied origins (e.g. osteoarthritis, rheumatoid arthritis, rheumatic disease, teno-synovitis and gout), headache, migraine and cluster headache, depression, anxiety, schizophrenia, stroke, traumatic brain injury, Alzheimer's disease, cerebral ischemia, amyotrophic lateral sclerosis, Huntington's disease, sensorineural hearing loss, tinnitus, glaucoma, neurological damage caused by epileptic seizures or by neurotoxin poisoning or by impairment of glucose and/or oxygen to the brain, vision loss caused by neurodegeneration of the visual pathway, Restless Leg Syndrome, multi-system atrophy, non-vascular headache, primary hyperalgesia, secondary hyperalgesia, primary allodynia, secondary allodynia, or other pain caused by central sensitization. Compounds of formula I may be used to prevent dyskinesias, particularly the side effects accompanying normal doses of L-Dopa. Furthermore, compounds of formula I may be used to decrease tolerance and/or dependence to opioid treatment of pain, and for treatment of withdrawal syndrome of e.g., alcohol, opioids, and cocaine.

The compounds of this invention are also useful for treating or preventing HIV- and HIV treatment-induced neuropathy, chronic pelvic pain, neuroma pain, complex regional pain syndrome, chronic arthritic pain and related neuralgias, treating or preventing chronic lower back pain, and treating or preventing pain resulting from, or associated with, traumatic nerve injury, nerve compression or entrapment, postherpetic neuralgia, trigeminal neuralgia, diabetic neuropathy, cancer and chemotherapy.

It is understood that compounds of this invention can be administered at prophylactically effective dosage levels to prevent the above-recited conditions, as well as to prevent other conditions mediated through the NMDA NR2B receptor.

Compounds of Formula I may be used in combination with other drugs that are used in the treatment/prevention/suppression or amelioration of the diseases or conditions for which compounds of Formula I are useful. Such other drugs may be administered, by a route and in an amount commonly used therefor, contemporaneously or sequentially with a compound of Formula I. When a compound of Formula I is used contemporaneously with one or more other drugs, a pharmaceutical composition containing such other drugs in addition to the compound of Formula I is preferred. Accordingly, the pharmaceutical compositions of the present invention include those that also contain one or more other active ingredients, in addition to a compound of Formula I. Examples of other active ingredients that may be combined with a compound of Formula I, either administered separately or in the same pharmaceutical compositions, include, but are not limited to: (1) non-steroidal anti-inflammatory agents; (2) COX-2 inhibitors; (3) bradykinin B 1 receptor antagonists; (4) sodium channel blockers and antagonists; (5) nitric oxide synthase (NOS) inhibitors; (6) glycine site antagonists; (7) potassium channel openers; (8) AMPA/kainate receptor antagonists; (9) calcium channel antagonists; (10) GABA-A receptor modulators (e.g., a GABA-A receptor agonist); (11) matrix metalloprotease (MMP) inhibitors; (12) thrombolytic agents; (13) opioids such as morphine; (14) neutrophil inhibitory factor (NIF); (15) L-Dopa; (16) carbidopa; (17) levodopa/carbidopa; (18) dopamine agonists such as bromocriptine, pergolide, pramipexole, ropinirole; (19) anticholinergics; (20) amantadine; (21) carbidopa; (22) catechol O-methyltransferase (“COMT”) inhibitors such as entacapone and tolcapone; (23) Monoamine oxidase B (“MAO-B”) inhibitors; (24) opiate agonists or antagonists; (25) 5HT receptor agonists or antagonists; (26) NMDA receptor agonists or antagonists; (27) NK1 antagonists; (28) selective serotonin reuptake inhibitors (“SSRI”) and/or selective serotonin and norepinephrine reuptake inhibitors (“SSNRI”); (29) tricyclic antidepressant drugs, (30) norepinephrine modulators; (31) lithium; (32) valproate; and (33) neurontin (gabapentin).

Creams, ointments, jellies, solutions, or suspensions containing the instant compounds can be employed for topical use. Mouth washes and gargles are included within the scope of topical use for the purposes of this invention.

A formulation intended for the oral administration to humans may conveniently contain from about 0.5 mg to about 5 g of active agent, compounded with an appropriate and convenient amount of carrier material which may vary from about 5 to about 95 percent of the total composition. Unit dosage forms can generally contain between from about 1 mg to about 1000 mg of the active ingredient.

The conditions recited herein can be treated or prevented by the administration of from about 0.01 mg to about 140 mg of the instant compounds per kilogram of body weight per day.

It is understood, however, that the specific dose level for any particular patient will depend upon a variety of factors. Such factors include the age, body weight, general health, sex, and diet of the patient. Other factors include the time and route of administration, rate of excretion, drug combination, and the type and severity of the particular disease undergoing therapy. For example, inflammatory pain may be effectively treated by the administration of from about 0.01 mg to about 75 mg of the present compound per kilogram of body weight per day, or alternatively about 0.5 mg to about 3.5 g per patient per day. Neuropathic pain may be effectively treated by the administration of from about 0.01 mg to about 125 mg of the present compound per kilogram of body weight per day, or alternatively about 0.5 mg to about 5.5 g per patient per day.

The abbreviations used herein are as follows unless specified otherwise:
TEA Triethylamine
NaOAc Sodium Acetate
DMF Dimethylformamide
Lawesson's Rgt [2,4-Bis(4-methoxyphenyl)-1,3-dithia-2,4-diphosphetane-2,4-disulfide]
Burgess Reagent (Methoxycarbonylsulfamoyl)triethylammonium hydroxide
DIPEA Diisopropylethylamine
EtOAc Ethyl acetate
TFA Trifluoroacetic acid
TFAA Trifluoroacetic anhydride
THF Tetrahydrofuran
DMAP 4-Dimethylaminopyridine
RT Room temperature
H Hours
Min Minutes
DCM Dichloromethane
MeCN Acetonitrile
MeOH Methanol
EtOH Ethanol
iPrOH 2-Propanol
n-BuOH 1-Butanol
EDC 1-(3-Dimethylaminopropyl)-3-ethylcarbodiimide hydrochloride
HOBt 1-Hydroxy-benzotriazole
NMP N-Methyl-2-pyrrolidinone
BOC—ON (2Z)-{[(tert-butoxycarbonyl)oxy]imino}(phenyl)acetonitrile
DAST (Diethylamino)sulfur trifluoride

The compounds of the present invention can be prepared readily according to the following Schemes and specific examples, or modifications thereof, using readily available starting materials, reagents and conventional synthesis procedures. In these reactions, it is also possible to make use of variants which are themselves known to those of ordinary skill in this art but are not mentioned in greater detail. The general procedures for making the compounds claimed in this invention can be readily understood and appreciated by one skilled in the art from viewing the following Schemes.

The preparation of final compounds I proceeds through structures such as those of intermediate II.

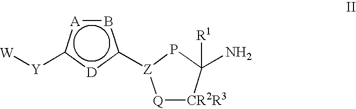

The synthesis of final compound I is accomplished through intermediate II as depicted in Scheme 1. In general, intermediate II is alkylated with 4-chloro-1H-pyrazolo[3,4-d]pyrimidine 1 under standard alkylation conditions (R. K. Robins, J. Amer. Chem. Soc. 78, 784-790 (1956)) such as hot alcoholic solvent, including isopropanol or 1-butanol, in the presence of base, including sodium carbonate or diisopropylethylamine. Intermediate II may also be alkylated with a suitably protected derivative of 1. Suitable protecting groups include alkoxymethyl derivatives such as N-tetrahydropyranyl, as illustrated by compound 2 and its isomer 3. Use of 2 and 3 in the alkylation reaction gives intermediates III and IV respectively. A suitable protecting group of this type is readily removed by treatment of intermediates III or IV with a protic acid, such as hydrochloric acid, and results in the formation of final compound I.

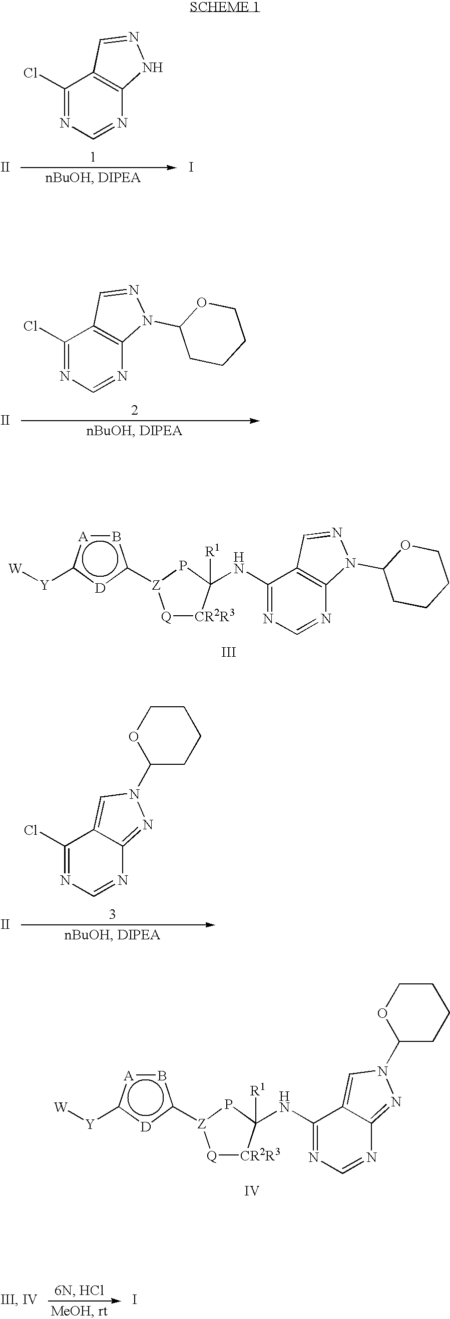

The synthesis of certain oxazole (exemplified by Example 5) containing compounds is depicted in Scheme 2. In the first step, a suitably protected amino-cycloalkyl-carboxylic acid 4 is converted to a primary amide 5 under standard conditions. The amide 5 can be converted into oxazole 6 by treatment with halo-ketone 7 followed by reprotection. Next, the nitrogen protecting group is removed under standard conditions such as acid hydrolysis. Intermediate 8 is converted to the final compound through the reaction sequence illustrated in Scheme 1.

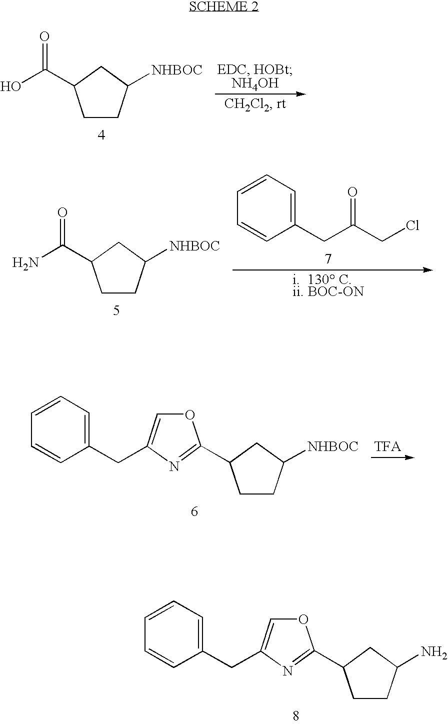

The synthesis of certain thiazole (exemplified by Example 1) containing compounds is depicted in Scheme 3. In the first step amide 5 is converted into thioamide 9 by treatment with Lawesson's reagent, and alkylated with halo-ketone 10 to yield thiazole 11. Intermediate 11 is converted to the final compound through the reaction sequences illustrated in Schemes 1 and 2.

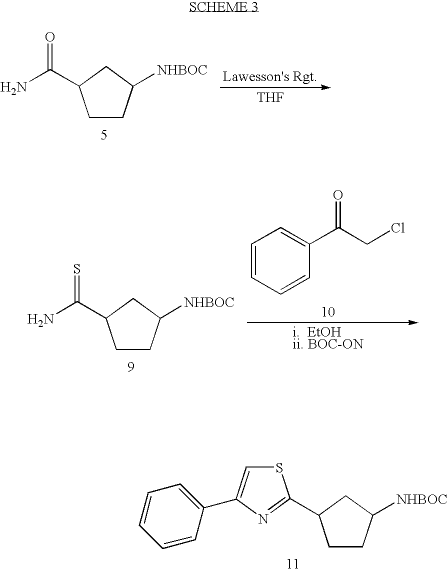

The synthesis of certain 1,2,4-oxadiazole containing compounds (exemplified by Example 18) is depicted in Scheme 4. In the first step, treatment of phenylacetonitrile 12 with hydroxylamine hydrochloride in the presence of base provides hydroxyamidine 13. The hydroxyamidine 13 is acylated with suitably protected amino acid 4 under standard conditions, such as EDC and HOBt, to give ester 14. Cyclodehydration of ester 14 occurs in the presence of base, such as sodium acetate or sodium carbonate, and yields oxadiazole 15. Intermediate 15 is converted to the final compound through the reaction sequences illustrated in Schemes 1 and 2.

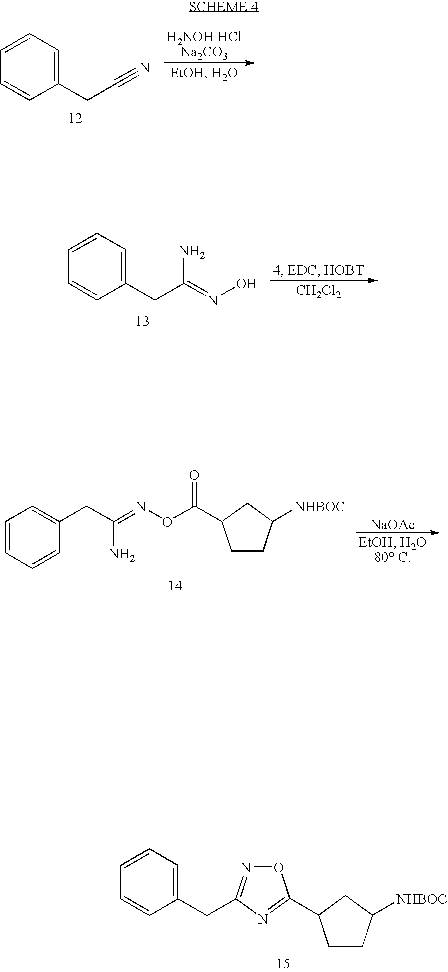

The synthesis of certain isomeric 1,2,4-oxadiazole containing compounds (exemplified by Example 43) is depicted in Scheme 5. Initially a suitably protected carboxamide 5 is dehydrated to nitrile 16 with a reagent such as Burgess reagent or trifluoroacetic anhydride in the presence of pyridine. Addition of hydroxylamine to 16 gives hydroxyamidine 17. Hydroxyamidine 17 is acylated with acid chloride 18 under standard conditions to give ester 19, which undergoes cyclodehydration in the presence of base to yield oxadiazole 20. Intermediate 20 is converted to the final compound through the reaction sequences illustrated in Schemes 1 and 2.

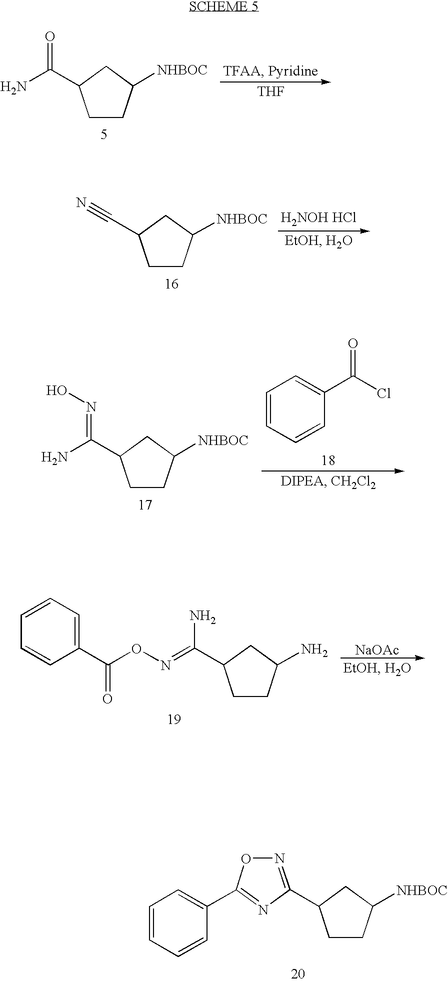

The synthesis of certain 1,3,4-oxadiazole containing compounds (exemplified by Example 47) is depicted in Scheme 6. In the first step, suitably protected carboxylic acid 4 is coupled under standard conditions to acylhydrazide 21, which is readily available by literature procedures. Cyclodehydration of 22 is accomplished with Burgess reagent to give oxadiazole 23. Intermediate 23 is converted to the final compound through the reaction sequences illustrated in Schemes 1 and 2.

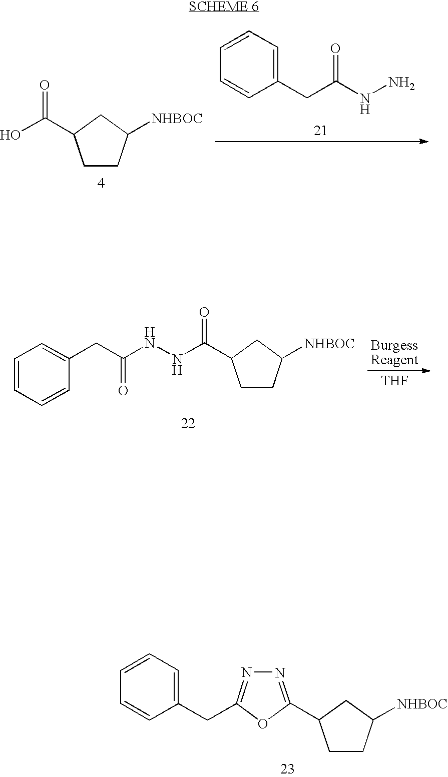

The synthesis of certain 1,3,4-thiadiazole containing compounds (exemplified by Example 48) is depicted in Scheme 7. In the first step, carboxylic acid 24 is coupled with hydrazine under standard conditions to give acylhydrazide 25. Carboxylic acid 4 is then coupled to 25 to give diacyhydrazide 26. Cyclodehydration of 26 is accomplished with Lawesson's reagent and the crude product treated with TFA to give thiadiazole 27. Intermediate 27 is converted to the final compound through the reaction sequence illustrated in Scheme 1.

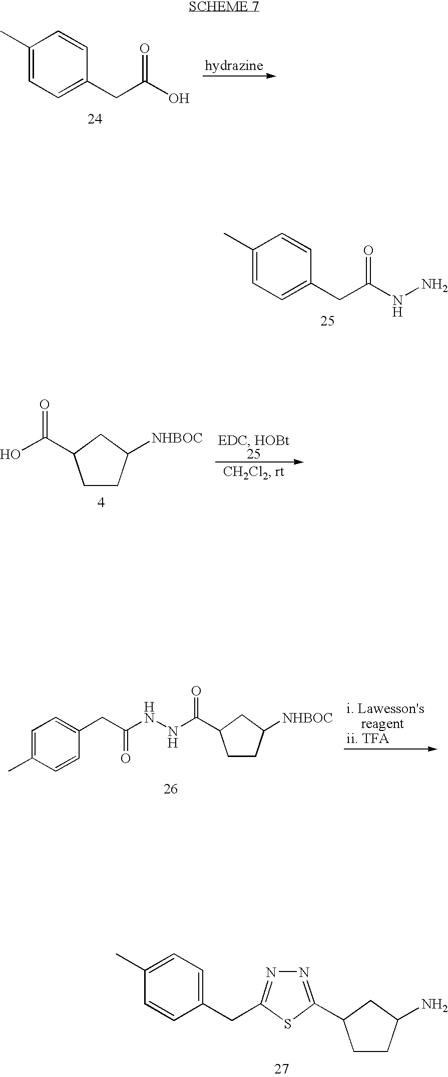

The synthesis of certain 1,2,4-triazole containing compounds (exemplified by Example 49) is depicted in Scheme 8. In the first step, carboxylic acid 4 is coupled to amine 28 to give amide 29. Treatment of 29 with Lawesson's reagent gives thioamide 30. Reaction of 30 with acylhydrazide 21 and mercuric acetate gives triazole 31. Intermediate 31 is converted to the final compound through the reaction sequences illustrated in Schemes 1 and 2.

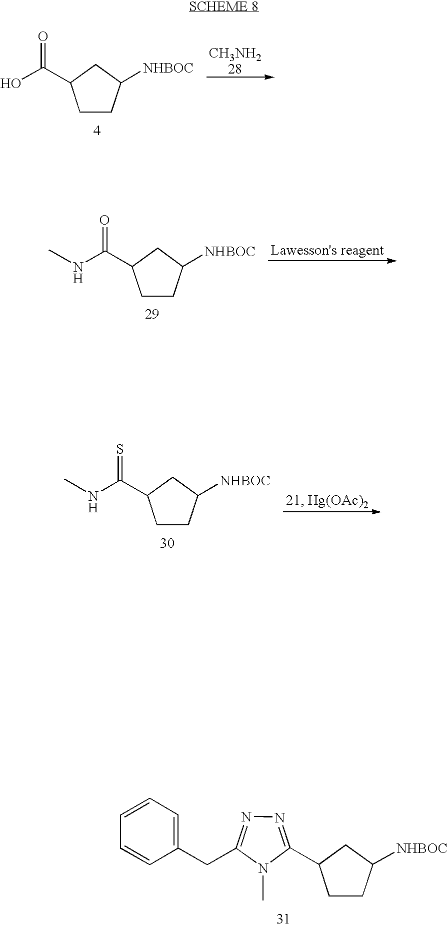

The synthesis of certain 5-amino-1,2,4-oxadiazole containing compounds (as exemplified by Example 51) is depicted in Scheme 9. In the first step, hydroxybenzamidine 32 is treated with trichloroacetic anhydride to give trichloromethyl-1,2,4-oxadiazole 33. Reaction of 33 with suitably protected aminopyrrolidine 34 gives 5-amino-1,2,4-oxadiazole 35. Intermediate 35 is converted to the final compound through the reaction sequences illustrated in Schemes 1 and 2.

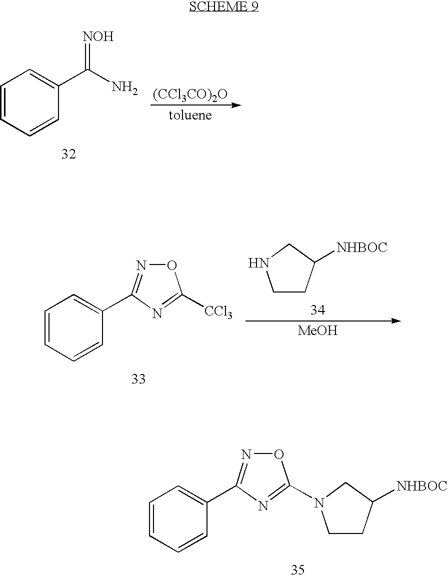

The synthesis of certain 2-amino-1,3,4-thiadiazole containing compounds (as exemplified by Example 57) is depicted in Scheme 10. In the first step, 4-methylphenylacetonitrile 36 is treated with thiosemicarbazide and TFA to give 2-amino-1,3,4-thiadiazole 37. Reaction of 37 under standard Sandmeyer conditions gives 2-chloro-1,3,4-thiadiazole 38. Reaction of 38 with pyrrolidine 34 in the presence of base gives the aminothiadiazole 39. Intermediate 39 is converted to the final compound through the reaction sequences illustrated in Schemes 1 and 2.

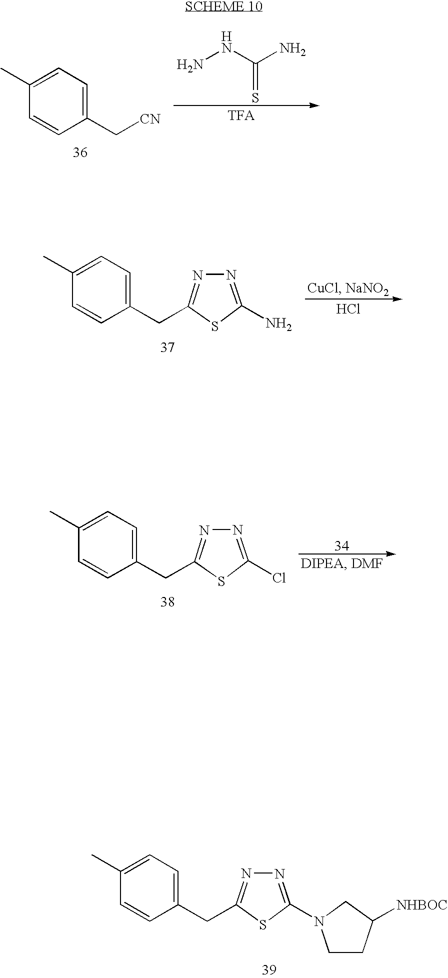

The synthesis of certain 3-amino-1,2,4-oxadiazole containing compounds (as exemplified by Example 58) is depicted in Scheme 11. In the first step, suitably protected amino-pyrrolidine 34 is converted to aminonitrile 40 by a reagent such as cyanogenbromide. Aminonitrile 40 is treated with hydroxylamine hydrochloride in the presence of base to give hydroxyamidine 41. Acylation of 41 with acid 42 under standard conditions, followed by cyclodehydration of ester 43 in the presence of base, yields amino-oxadiazole 44. Intermediate 44 is converted to the final compound through the reaction sequences illustrated in Schemes 1 and 2.

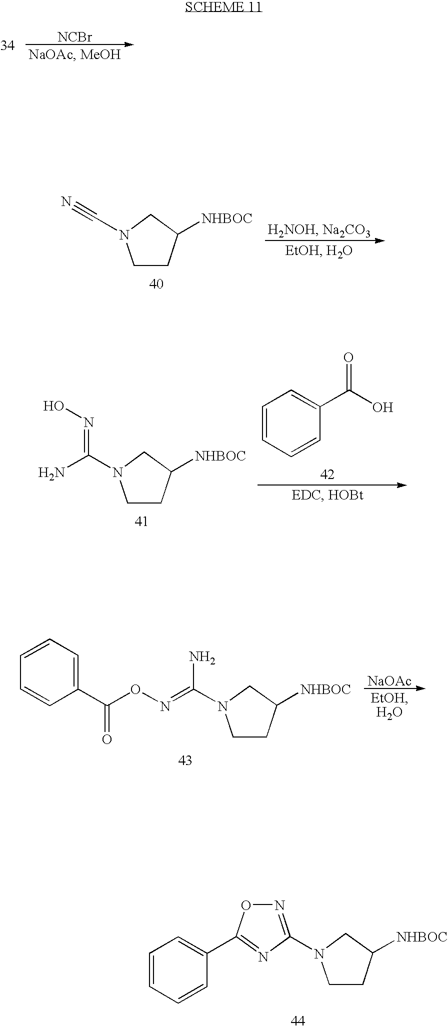

The synthesis of certain 3-amino-1,2,4-triazole containing compounds (as exemplified by Example 81) is depicted in Scheme 12. In the first step, benzotriazole 45 is reacted with cyanogen bromide in the presence of base to give compound 46. Compound 46 is then reacted with suitably protected amino-pyrrolidine 34 to give pyrrolidine derivative 47. Acylation of 47 with acid chloride 48 under standard conditions, followed by treatment with hydrazine 49 and then TFA yields triazole 50. Intermediate 50 is converted to the final compound through the reaction sequence illustrated in Scheme 1.

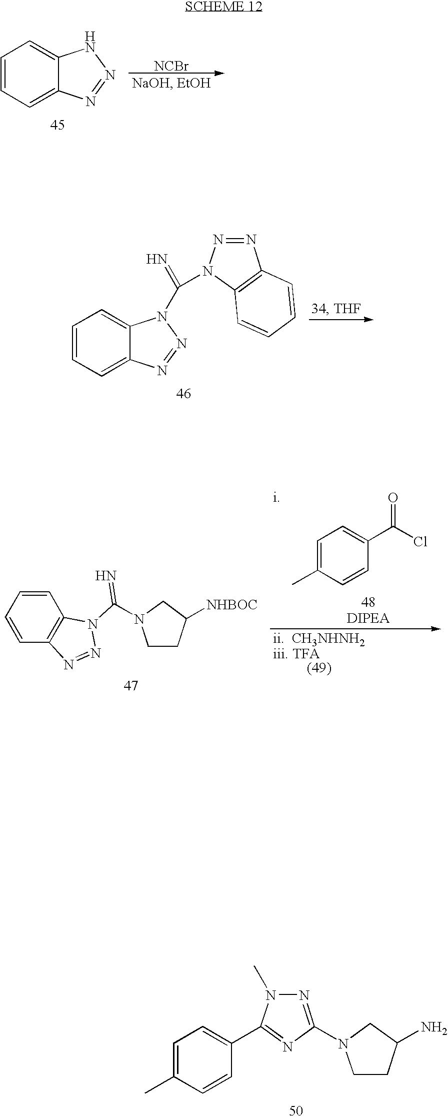

In some cases the final product may be further modified, for example, by manipulation of substituents. These manipulations may include, but are not limited to, reduction, oxidation, alkylation, acylation, halogenation and hydrolysis reactions which are commonly known to those skilled in the art.

In some cases the order of carrying out the foregoing reaction schemes may be varied to facilitate the reaction or to avoid unwanted reaction products. The following examples are provided so that the invention might be more fully understood. These examples are illustrative only and should not be construed as limiting the invention in any way.

### EXAMPLE 1

### N-[(1S,3R)-3-(4-phenyl-1,3-thiazol-2-yl)cyclopentyl]-1H-pyrazolo[3,4-d]pyrimidin-4-amine

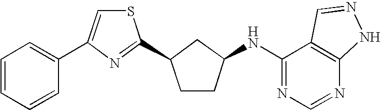

### Step A: tert-butyl [(1S,3R)-3-(aminocarbonyl)cyclopentyl]carbamate

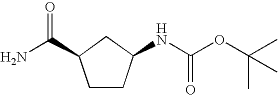

To a solution of (1R,3S)-3-[(tert-butoxycarbonyl)amino]cyclopentanecarboxylic acid (2.00 g, 8.72 mmol) and HOBt (3.54 g, 26.2 mmol) in DMF (25 mL) at room temperature was added EDC (5.02 g, 26.2 mmol). After 30 minutes, ammonium hydroxide (5.4 mL) was added to the reaction. After 96 h, the reaction was poured into ethyl acetate. The organic layer was washed with water and brine, dried over sodium sulfate, filtered and concentrated to give the title compound (1.64 g) as a white solid. This material was sufficiently pure for the next step. ¹H NMR (400 MHz, DMSO-d₆): 7.27 (s, 1H), 6.83 (d, J=7.6 Hz, 1H), 6.74 (s, 1H), 3.76-3.72 (m, 1H), 2.60-2.56 (m, 1H), 1.99-1.92 (m, 1H), 1.75-1.67 (m, 3H), 1.49-1.42 (m, 2H), 1.37 (s, 9H).

### Step B: tert-butyl [(1S,3R) and (1S,3S)-3-(aminocarbonothioyl)cyclopentyl]carbamate

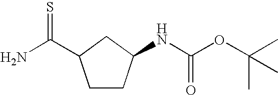

To a suspension of tert-butyl [(1S,3R)-3-(aminocarbonyl)cyclopentyl]carbamate (1.64 g, 7.18 mmol) in THF (30 mL) at room temperature was added Lawesson's reagent (1.89 g, 4.67 mmol). After 17 h, the reaction was poured into ethyl acetate. The organic layer was washed with saturated sodium bicarbonate and brine, dried over sodium sulfate, filtered and concentrated. Purification by silica gel chromatography (15% ethyl acetate/hexanes→90% ethyl acetate/hexanes) gave a mixture of diastereomers of the title compound (1.22 g) as a white solid. MS 245 (M+1) and 189 (M−55).

### Step C: tert-butyl [(1S,3R) and (1S,3S)-3-(4-phenyl-1,3-thiazol-2-yl)cyclopentyl]carbamate

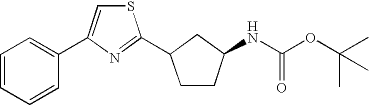

To a solution of tert-butyl [(1S,3R) and (1S,3S)-3-(aminocarbonothioyl)cyclopentyl]carbamate (1.22 g, 4.99 mmol) in ethanol (20 mL) was added 2-bromo-1-phenylethanone (994 mg, 4.99 mmol) and the resulting solution was heated to 85° C. After 3.5 hours, the reaction was cooled to room temperature and concentrated. To a suspension of the crude residue in triethylamine (10 mL) was added (2Z)-{[(tert-butoxycarbonyl)oxy]imino}(phenyl)acetonitrile (1.23 g, 4.99 mmol) and the reaction was maintained at room temperature for 96 hours. The reaction mixture was concentrated and dissolved in ethyl acetate. The organic layer was washed with 1M sodium hydroxide, water and brine, dried over sodium sulfate, filtered and concentrated. Purification by silica gel chromatography (1% ethyl acetate/hexanes→40% ethyl acetate/hexanes) gave the title compound as a mixture of diastereomers (1.51 g). MS 345 (M+1).

### Step D: (1S,3R) and (1S,3S)-3-(4-phenyl-1,3-thiazol-2-yl)cyclopentanamine

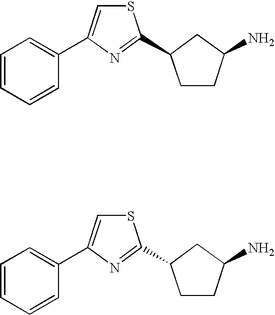

To a solution of tert-butyl [(1S,3R) and (1S,3S)-3-(4-phenyl-1,3-thiazol-2-yl)cyclopentyl]carbamate (1.51 g, 4.39 mmol) in methylene chloride (5 mL) was added trifluoroacetic acid (0.98 mL, 13.17 mmol) at room temperature. After 17 hours, the reaction was partially concentrated and trifluoroacetic acid (0.5 mL) was added at room temperature. After 6 hours, the reaction mixture was concentrated under reduced pressure. The crude material was loaded onto an SCX ion exchange resin as a solution in acetonitrile/water (1:1) and eluted with ammonia in ethanol solution to give an orange oil. Purification by chiral HPLC (Chiralcel OD, 30% isopropanol/hexanes→60% isopropanol/hexanes) and treatment with anhydrous hydrochloric acid gave the hydrochloride salt of the title compounds.

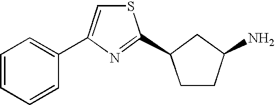

(1S,3R)-3-(4-phenyl-1,3-thiazol-2-yl)cyclopentanamine: 718 mg, HRMS (M+H⁺): calculated=245.1107, observed=245.1108; ¹H NMR (400 MHz, DMSO-d₆): 8.17 (br s, 2H), 8.00 (s, 1H), 7.96 (s, 1H), 7.94 (s, 1H), 7.44 (t, J=7.5 Hz, 2H), 7.34 (t, J=7.3 Hz, 1H), 3.67-3.58 (m, 2H), 2.59-2.55 (m, 1H), 2.20-1.90 (m, 4H), 1.84-1.80 (m, 1H).

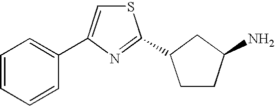

(1S,3S)-3-(4-phenyl-1,3-thiazol-2-yl)cyclopentanamine: 469 mg, HRMS (M+H⁺): calculated=245.1107, observed=245.1104; ¹H NMR (400 MHz, DMSO-d₆): 8.13 (br s, 2H), 8.00 (s, 1H), 7.95 (s, 1H), 7.93 (s, 1H), 7.44 (t, J=7.7 Hz, 2H), 7.34 (t, J=7.3 Hz, 1H), 3.86-3.76 (m, 2H), 2.35-2.17 (m, 4H), 1.91-1.82 (m, 1H), 1.79-1.72 (m, 1H).

### Step E: N-[(1S,3R)-3-(4-phenyl-1,3-thiazol-2-yl)cyclopentyl]-1H-pyrazolo[3,4-d]pyrimidin-4-amine

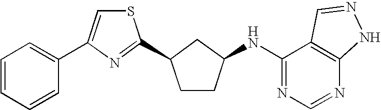

To a solution of (1S,3R)-3-(4-phenyl-1,3-thiazol-2-yl)cyclopentanamine (100 mg, 0.356 mmol) in isopropanol (5 mL) was added DIPEA (2 mL) and 4-chloro-1-(tetrahydro-pyran-2-yl)-1H-pyrazolo[3,4-d]pyrimidine (85 mg, 0.356 mmol) and the solution was heated at 85° C. for 4 hours. The mixture was cooled and concentrated under reduced pressure. The resulting residue was dissolved in methanol (3 mL) and 6N HCl (0.5 mL) and was heated at 60° C. for 2 hours, at which time the reaction was cooled, quenched with saturated sodium bicarbonate and concentrated under reduced pressure. The residue was treated with saturated sodium bicarbonate, extracted with ethyl acetate, and the organic layer was dried over sodium sulfate, filtered and concentrated to dryness under reduced pressure. Purification by silica gel chromatography (2% isopropanol/methylene chloride→30% isopropanol/methylene chloride) gave the title compound (95 mg) as a white solid. HRMS (M+H⁺): calculated=363.1387, observed=363.1380; ¹H NMR (400 MHz, DMSO-d₆): 13.37 (s, 1H), 8.24-8.22 (m, 2H), 8.12 (s, 1H), 7.97-7.94 (m, 3H), 7.45-7.35 (m, 2H), 7.35-7.32 (m, 1H), 4.70 (br s, 1H), 3.67-3.64 (m, 1H), 2.70-2.63 (m, 1H), 2.23 (m, 2H), 2.09-1.94 (m, 2H), 1.81 (m, 1H).

### EXAMPLE 2

### N-[(1S,3S)-3-(4-phenyl-1,3-thiazol-2-yl)cyclopentyl]-1H-pyrazolo[3,4-d]pyrimidin-4-amine

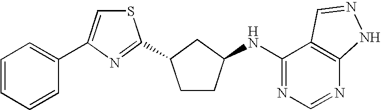

To a solution of (1S,3S)-3-(4-phenyl-1,3-thiazol-2-yl)cyclopentanamine (100 mg, 0.356 mmol; from Example 1, Step D) in isopropanol (5 mL) was added DIPEA (2 mL) and 4-chloro-1-(tetrahydro-pyran-2-yl)-1H-pyrazolo[3,4-d]pyrimidine (85 mg, 0.356 mmol) and the solution was heated at 85° C. for 4 hours. The mixture was cooled and concentrated under reduced pressure. The resulting residue was dissolved in methanol (3 mL) and 6N HCl (0.5 mL) and was heated at 60° C. for 2 hours, at which time the reaction was cooled, quenched with saturated sodium bicarbonate and concentrated under reduced pressure. The residue was treated with saturated sodium bicarbonate, extracted with ethyl acetate, and the organic layer was dried over sodium sulfate, filtered and concentrated to dryness under reduced pressure. Purification by silica gel chromatography (2% isopropanol/methylene chloride→30% isopropanol/methylene chloride) gave the title compound (78 mg) as a white solid. HRMS (M+H⁺): calculated=363.1387, observed=363.1379; ¹H NMR (400 MHz, DMSO-d₆): 13.38 (s, 1H), 8.22-8.16 (m, 3H), 7.99-7.95 (m, 3H), 7.44 (t, J=7.5 Hz, 2H), 7.34 (t, J=7.3 Hz, 1H), 4.76 (br s, 1H), 3.85-3.81 (m, 1H), 2.40-2.30 (m, 3H), 2.24-2.17 (m, 1H), 1.96-1.91 (m, 1H), 1.79 (m, 1H).

### EXAMPLE 3

### N-[(1S,3R)-3-(4-benzyl-1,3-thiazol-2-yl)cyclopentyl]-1H-pyrazolo[3,4-d]pyrimidin-4-amine

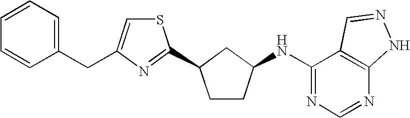

### Step A: tert-butyl [(1S,3R) and (1S,3R)-benzyl-1,3-thiazol-2-yl)cyclopentyl]carbamate

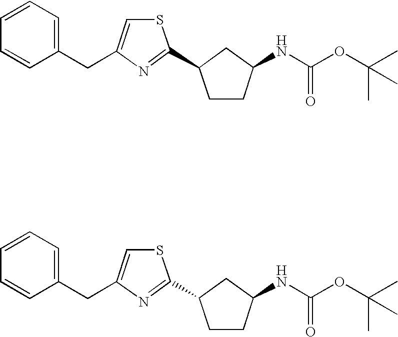

To a solution of tert-butyl [(1S,3R) and (1S,3R)-3-(aminocarbonothioyl)cyclopentyl]carbamate (262 mg, 1.07 mmol; from Example 1, Step B) in ethanol (5 mL) was added 1-chloro-3-phenylacetone (181 mg, 1.07 mmol; McPhee, W. D.; Klingsberg, E. Organic Syntheses 26, 13-15 (1946) and McPhee, W. D.; Klingsberg, E. Journal of the American Chemical Society 66, 1132-1136 (1944)) and the resulting solution was heated to 85° C. After 3 hours, the reaction was cooled to room temperature, concentrated and diluted with THF (1 mL). Triethylamine (2 mL) and BOC—ON (264 mg, 1.07 mmol) were added and the reaction was maintained at room temperature for 16 hours. The reaction mixture was concentrated and dissolved in ethyl acetate. The organic layer was washed with 1M sodium hydroxide, water, brine, dried over sodium sulfate, filtered and concentrated. Purification by silica gel chromatography (1% ethyl acetate/hexanes→50% ethyl acetate/hexanes) gave the title compounds.

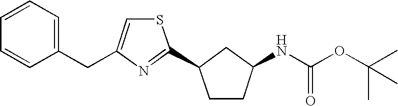

tert-butyl [(1S,3R)-3-(4-benzyl-1,3-thiazol-2-yl)cyclopentyl]carbamate: 108 mg, MS 359 (M+1); ¹H NMR (400 MHz, CDCl₃): 7.30-7.23 (m, 5H), 6.58 (s, 1H), 5.81 (s, 1H), 4.16 (s, 1H), 4.09 (s, 2H), 3.52 (m, 1H), 2.44-2.41 (m, 1H), 2.19-2.16 (m, 1H), 2.02-1.91 (m, 2H), 1.83-1.69 (m, 2H), 1.46 (s, 9H).

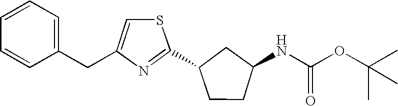

tert-butyl [(1S,3S)-3-(4-benzyl-1,3-thiazol-2-yl)cyclopentyl]carbamate: 115 mg, MS 359 (M+1); ¹H NMR (400 MHz, CDCl₃): 7.33-7.23 (m, 5H), 6.57 (s, 1H), 4.57 (s, 1H), 4.18 (s, 1H), 4.09 (s, 2H), 3.62-3.57 (m, 1H), 2.29-2.18 (m, 3H), 2.07 (m, 1H), 1.89-1.84 (m, 1H), 1.53 (m, 1H), 1.45 (s, 9H).

### Step B: [(1S,3R)-3-(4-benzyl-1,3-thiazol-2-yl)cyclopentyl]amine

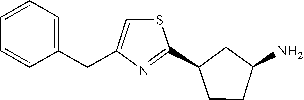

To tert-butyl [(1S,3R)-3-(4-benzyl-1,3-thiazol-2-yl)cyclopentyl]carbamate (108 mg, 0.301 mmol) was added trifluoroacetic acid (0.5 mL) and the resulting solution was stirred at room temperature. After 20 minutes, the reaction was concentrated and the residue was dissolved in ethyl acetate. The solution was treated with anhydrous hydrochloric acid and filtered to give the hydrochloride salt of the title compound (94 mg). HRMS (M+H⁺): calculated=259.1264, observed=259.1271; ¹H NMR (400 MHz, DMSO-d₆): 8.00 (s, 2H), 7.33-7.20 (m, 5H), 7.15 (s, 1H), 4.03 (s, 2H), 3.62 (m, 1H), 3.52-3.48 (m, 1H), 2.13-2.03 (m, 3H), 1.93-1.72 (m, 3H).

### Step C: N-[(1S,3R)-3-(4-benzyl-1,3-thiazol-2-yl)cyclopentyl]-1H-pyrazolo[3,4-d]pyrimidin-4-amine

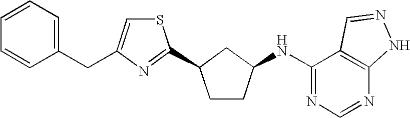

To a solution of [(1S,3R)-3-(4-benzyl-1,3-thiazol-2-yl)cyclopentyl]amine (81 mg, 0.24 mmol) in 1-butanol (0.5 mL) was added DIPEA (0.5 mL) and 4-chloro-1-(tetrahydro-pyran-2-yl)-1H-pyrazolo[3,4-d]pyrimidine (58 mg, 0.24 mmol) and the solution was heated at 150° C. for 10 minutes under microwave irradiation. The mixture was cooled and concentrated under reduced pressure. The resulting residue was dissolved in methanol (2 mL) and 6N HCl (1 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (65 mg) as a white solid. HRMS (M+H⁺): calculated=377.1543, observed=377.1554; ¹H NMR (400 MHz, DMSO-d₆): 13.36 (s, 1H), 8.22 (s, 2H), 8.12 (s, 1H), 7.32-7.19 (m, 5H), 7.10 (s, 1H), 4.66 (m, 1H), 4.03 (s, 2H), 3.54 (s, 1H), 2.58 (m, 1H), 2.21-2.14 (m, 2H), 1.97-1.84 (m, 2H), 1.74 (m 1H).

### EXAMPLE 4

### N-[(1S,3S)-3-(4-benzyl-1,3-thiazol-2-yl)cyclopentyl]-1H-pyrazolo[3,4-d]pyrimidin-4-amine

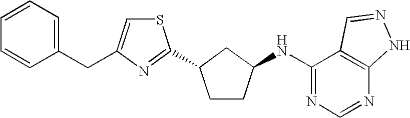

### Step A: [(1S,3S)-3-(4-benzyl-1,3-thiazol-2-yl)cyclopentyl]amine

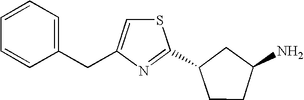

To tert-butyl [(1S,3S)-3-(4-benzyl-1,3-thiazol-2-yl)cyclopentyl]carbamate (118 mg, 0.321 mmol; from Example 3, Step A) was added trifluoroacetic acid (0.5 mL) and the resulting solution was stirred at room temperature. After 20 minutes, the reaction was concentrated and the residue was dissolved in ethyl acetate. The solution was treated with anhydrous hydrochloric acid and filtered to give the hydrochloride salt of the title compound (112 mg). HRMS (M+H⁺): calculated=259.1264, observed=259.1271; ¹H NMR (400 MHz, DMSO-d₆): 7.82 (s, 2H), 7.30-7.24 (m, 5H), 7.13 (s, 1H), 4.02 (s, 2H), 3.69 (m, 2H), 2.24-2.02 (m, 4H), 1.80-1.60 (m, 2H).

### Step B: N-[(1S,3S)-3-(4-benzyl-1,3-thiazol-2-yl)cyclopentyl]-1H-pyrazolo[3,4-d]pyrimidin-4-amine

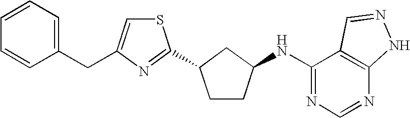

To a solution of [(1S,3S)-3-(4-benzyl-1,3-thiazol-2-yl)cyclopentyl]amine (95 mg, 0.287 mmol) in 1-butanol (0.5 mL) was added DIPEA (0.5 mL) and 4-chloro-1-(tetrahydro-pyran-2-yl)-1H-pyrazolo[3,4-d]pyrimidine (68 mg, 0.244 mmol) and the solution was heated at 150° C. for 10 minutes under microwave irradiation. The mixture was cooled and concentrated under reduced pressure. The resulting residue was dissolved in methanol (2 mL) and 6N HCl (1 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (71 mg) as a white solid. HRMS (M+H⁺): calculated=377.1543, observed=377.1556; ¹H NMR (400 MHz, DMSO-d₆): 13.36 (s, 1H), 8.21 (s, 1H), 8.13 (s, 2H), 7.32-7.18 (m, 5H), 7.10 (s, 1H), 4.69 (m, 1H), 4.03 (s, 2H), 3.71 (m, 1H), 2.32-2.13 (m, 4H), 1.85-1.73 (m, 2H).

### EXAMPLE 5

### N-[(1S,3S)-3-(4-benzyl-1,3-oxazol-2-yl)cyclopentyl]-1H-pyrazolo[3,4-d]pyrimidin-4-amine

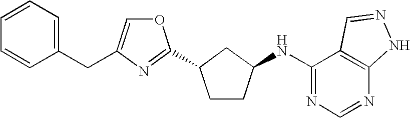

### Step A: tert-butyl [(1S,3S)-3-(aminocarbonyl)cyclopentyl]carbamate

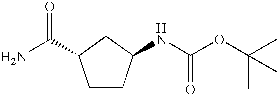

To a solution of (1S,3S)-3-[(tert-butoxycarbonyl)amino]cyclopentanecarboxylic acid (480 mg, 2.09 mmol) and HOBt (849 mg, 6.28 mmol) in DMF (6 mL) at room temperature was added EDC (1.20 g, 6.28 mmol). After 30 minutes, ammonium hydroxide (1.3 mL) was added to the reaction. After 72 h, the reaction was poured into ethyl acetate. The organic layer was washed with water, brine, dried over sodium sulfate, filtered and concentrated to give the title compound (306 mg) as a white solid. This material was sufficiently pure for the next step. ¹H NMR (400 MHz, DMSO-d6): 7.20 (s, 1H), 6.81 (d, J=8.0 Hz, 1H), 6.67 (s, 1H), 3.82-3.80 (m, 1H), 2.69-2.64 (m, 1H), 1.89-1.76 (m, 3H), 1.61-1.52 (m, 2H), 1.37 (s, 9H).

### Step B: tert-butyl [(1S,3S)-3-(4-benzyl-1,3-oxazol-2-yl)cyclopentyl]carbamate

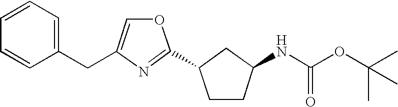

A mixture of tert-butyl [(1S,3S)-3-(aminocarbonyl)cyclopentyl]carbamate (200 mg, 0.876 mmol) and 1-chloro-3-phenylacetone (148 mg, 0.876 mmol) was heated to 130° C. After 2 hours, the reaction was cooled to room temperature, dissolved in 1M sodium hydroxide and extracted with ethyl acetate. The organic layer dried over sodium sulfate, filtered and concentrated. To a solution of the crude residue in triethylamine (3 mL) was added BOC—ON (216 mg, 0.876 mmol) at room temperature. After 3 hours, the reaction was concentrated and dissolved in ethyl acetate. The organic layer was washed with 1M sodium hydroxide, water, brine, dried over sodium sulfate, filtered and concentrated. Purification by silica gel chromatography (1% ethyl acetate/hexanes→45% ethyl acetate/hexanes) gave the title compound (28 mg) as an oil. MS 343 (M+1); ¹H NMR (400 MHz, DMSO-d₆): 7.64 (s, 1H), 7.31-7.18 (m, 5H), 6.95 (s, 1H), 3.92 (s, 1H), 3.76 (s, 2H), 2.08-1.66 (m, 5H), 1.55-1.46 (m, 1H), 1.37 (s, 9H).

### Step C: [(1S,3S)-3-(4-benzyl-1,3-oxazol-2-yl)cyclopentyl]amine

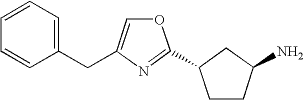

To tert-butyl [(1S,3S)-3-(4-benzyl-1,3-oxazol-2-yl)cyclopentyl]carbamate (28 mg, 0.082 mmol) was added trifluoroacetic acid (0.75 mL) and the resulting solution was stirred at room temperature. After 30 minutes, the reaction was concentrated and the residue was dissolved in ethyl acetate. The solution was treated with anhydrous hydrochloric acid and concentrated to give the hydrochloride salt of the title compound (40 mg) as a yellow oil. MS 243 (M+1).

### Step D: N-[(1S,3S)-3-(4-benzyl-1,3-oxazol-2-yl)cyclopentyl]-1H-pyrazolo[3,4-d]pyrimidin-4-amine

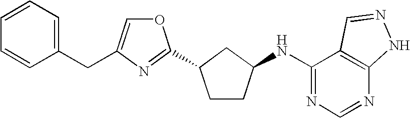

To a solution of [(1S,3S)-3-(4-benzyl-1,3-oxazol-2-yl)cyclopentyl]amine (23 mg, 0.083 mmol) in isopropanol (3 mL) was added DIPEA (1 mL) and 4-chloro-1-(tetrahydro-pyran-2-yl)-1H-pyrazolo[3,4-d]pyrimidine (20 mg, 0.083 mmol) and the solution was heated at 70° C. After 15 hours, the mixture was cooled and concentrated under reduced pressure. Purification by silica gel chromatography (1% isopropanol/methylene chloride→15% isopropanol/methylene chloride) gave the THP-protected compound (22 mg) as an oil; MS 445 (M+1). The resulting residue was dissolved in methanol (3 mL) and 6N HCl (0.5 mL) and was heated at 60° C. for 1 hour, at which time the reaction was cooled and quenched with saturated sodium bicarbonate. The solution was partially concentrated under reduced pressure and the resulting solid was filtered to give the title compound (9.4 mg) as a white solid. HRMS (M+H⁺): calculated=361.1772, observed=361.1769; ¹H NMR (400 MHz, DMSO-d₆): 13.4 (s, 1H), 8.20-8.11 (m, 3H), 7.69 (s, 1H), 7.32-7.19 (m, 5H), 4.64 (br s, 1H), 3.79 (s, 2H), 3.49 (m, 1H), 2.25-2.17 (m, 3H), 2.05-1.83 (m, 2H), 1.70 (m, 1H).

### EXAMPLE 6

### N-[(1S,3R)-3-(3-phenyl-1,2,4-oxadiazol-5-yl)cyclopentyl]-1H-pyrazolo[3,4-d]pyrimidin-4-amine

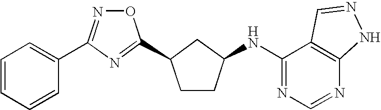

### Step A: tert-butyl {(1S,3R)-3-[({[(1Z)-amino(phenyl)methylene]amino}oxy)carbonyl]cyclopentyl}carbamate

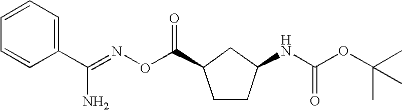

To a solution of (1R,3S)-3-[(tert-butoxycarbonyl)amino]cyclopentanecarboxylic acid (500 mg, 2.18 mmol) in methylene chloride (10 mL) at room temperature was added HOBt (334 mg, 2.18 mmol), EDC (472 mg, 2.29 mmol) and N′-hydroxybenzenecarboximidamide (327 mg, 2.40 mmol). After 21 h, the reaction was poured into water and extracted with methylene chloride. The organic layer was dried over sodium sulfate, filtered and concentrated. Purification by silica gel chromatography (50% ethyl acetate/hexanes→100% ethyl acetate/hexanes) gave the title compound (600 mg) as a white solid. MS 348 (M+H⁺); ¹H NMR (400 MHz, DMSO-d₆): 7.70 (d, J=7.4 Hz, 2H), 7.50-7.43 (m, 3H), 6.89 (d, J=7.2 Hz, 1H), 6.74 (s, 2H), 3.80 (m, 1H), 2.97-2.93 (m, 1H), 2.22-2.19 (m, 1H), 1.90-1.80 (m, 3H), 1.65-1.47 (m, 2H), 1.38 (s, 9H).

### Step B: tert-butyl [(1S,3R)-3-(3-phenyl-1,2,4-oxadiazol-5-yl)cyclopentyl]carbamate

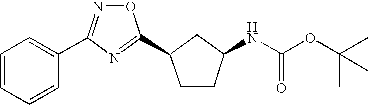

To a solution of tert-butyl {(1S,3R)-3-[({[(1Z)-amino(phenyl)methylene]amino}oxy)carbonyl]cyclopentyl}carbamate (600 mg, 1.73 mmol) in ethanol (20 mL) and water (6 mL) was added sodium acetate (142 mg, 1.73 mmol) and the resulting solution was heated to 85° C. After 3 hours, the reaction was concentrated. Purification by silica gel chromatography (5% ethyl acetate/hexanes→50% ethyl acetate/hexanes) gave the title compound (435 mg) as a white solid. HRMS (M+H⁺): calculated=352.1631, observed=352.1632; ¹H NMR (400 MHz, DMSO-d₆): 8.02-7.99 (m, 2H), 7.60-7.54 (m, 1H), 7.01 (d, J=9.6 Hz, 2H), 3.94-3.92 (m, 1H), 3.56-3.50 (m, 1H), 2.45-2.38 (m, 10H), 2.12-1.79 (m, 4H), 1.65-1.59 (m, 1H), 1.38 (s, 9H).

### Step C: [(1S,3R)-3-(3-phenyl-1,2,4-oxadiazol-5-yl)cyclopentyl]amine

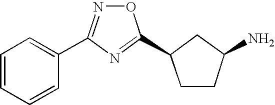

To tert-butyl [(1S,3R)-3-(3-phenyl-1,2,4-oxadiazol-5-yl)cyclopentyl]carbamate (400 mg, 1.21 mmol) was added trifluoroacetic acid (1.5 mL) and the resulting solution was stirred at room temperature. After 30 minutes, the reaction was concentrated and the residue was dissolved in ethyl acetate. The solution was treated with anhydrous hydrochloric acid and concentrated to give the hydrochloride salt of the title compound (347 mg) as a white solid. HRMS (M+H⁺): calculated=230.1288, observed=230.1294; ¹H NMR (400 MHz, DMSO-d₆): 8.12 (s, 3H), 8.03-8.00 (m, 2H), 7.61-7.56 (m, 3H), 3.66-3.59 (m, 2H), 2.62-2.58 (m, 1H), 2.50-1.99 (m, 4H), 1.82 (m, 1H).

### Step D: N-[(1S,3R)-3-(3-phenyl-1,2,4-oxadiazol-5-yl)cyclopentyl]-1H-pyrazolo[3,4-d]pyrimidin-4-amine

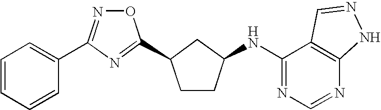

To a solution of [(1S,3R)-3-(3-phenyl-1,2,4-oxadiazol-5-yl)cyclopentyl]amine (70 mg, 0.263 mmol) in isopropanol (3 mL) was added DIPEA (1 mL) and 4-chloro-1-(tetrahydro-pyran-2-yl)-1H-pyrazolo[3,4-d]pyrimidine (66 mg, 0.277 mmol) and the solution was heated at 85° C. for 1 hour. The mixture was cooled and concentrated under reduced pressure. The resulting residue was dissolved in methanol (3 mL) and 6N HCl (0.5 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (63 mg) as an off-white solid. HRMS (M+H⁺): calculated=348.1567, observed=348.1556; ¹H NMR (400 MHz, DMSO-d₆): 13.39 (s, 1H), 8.25-8.21 (m, 2H), 8.12 (s, 1H), 8.01-7.99 (m, 2H), 7.60-7.55 (m, 3H), 4.71 (m, 1H), 3.69-3.65 (m, 1H), 2.71-2.63 (m, 1H), 2.27-2.13 (m, 3H), 2.08-2.00 (m, 1H), 1.82 (m, 1H).

### EXAMPLE 7

### N-[(1S,3S)-3-(3-phenyl-1,2,4-oxadiazol-5-yl)cyclopentyl]-1H-pyrazolo[3,4-d]pyrimidin-4-amine

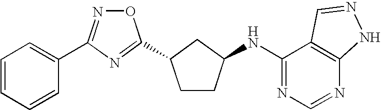

### Step A: tert-butyl {(1S,3S)-3-[({[(1Z)-amino(phenyl)methylene]amino}oxy)carbonyl]cyclopentyl}carbamate

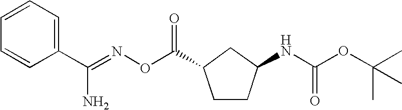

To a solution of (1S,3S)-3-[(tert-butoxycarbonyl)amino]cyclopentanecarboxylic acid (200 mg, 0.872 mmol) in methylene chloride (10 mL) at room temperature was added HOBt (134 mg, 0.872 mmol), EDC (189 mg, 0.916 mmol) and N′-hydroxybenzenecarboximidamide (131 mg, 0.960 mmol). After 3 h, the reaction was poured into water and extracted with methylene chloride. The organic layer was dried over sodium sulfate, filtered and concentrated. Purification by silica gel chromatography (30% ethyl acetate/hexanes→100% ethyl acetate/hexanes) gave the title compound (277 mg) as a white solid. MS 348 (M+1); ¹H NMR (300 MHz, DMSO-d₆): 7.71-7.68 (m, 2H), 7.50-7.41 (m, 3H), 6.91 (d, J=6.6 Hz, 1H), 6.75 (s, 2H), 3.88 (m, 1H), 3.12-3.07 (m, 1H), 2.05-1.43 (m, 6H), 1.32 (s, 9H).

### Step B: tert-butyl [(1S,3S)-3-(3-phenyl-1,2,4-oxadiazol-5-yl)cyclopentyl]carbamate

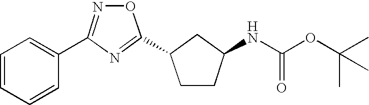

To a solution of tert-butyl {(1S,3S)-3-[({[(1Z)-amino(phenyl)methylene]amino}oxy)carbonyl]cyclopentyl}carbamate (277 mg, 0.797 mmol) in ethanol (10 mL) and water (2.5 mL) was added sodium acetate (65 mg, 0.797 mmol) and the resulting solution was heated to 85° C. After 5 hours, the reaction was cooled to room temperature, concentrated, suspended in water and extracted with ethyl acetate. The organic layer was dried over sodium sulfate, filtered and concentrated to give the title compound (262 mg) as a white solid. This material was sufficiently pure for the next step. MS 330 (M+1).

### Step C: [(1S,3S)-3-(3-phenyl-1,2,4-oxadiazol-5-yl)cyclopentyl]amine

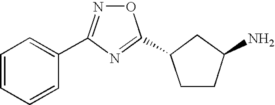

To tert-butyl [(1S,3S)-3-(3-phenyl-1,2,4-oxadiazol-5-yl)cyclopentyl]carbamate (262 mg, 0.795 mmol) was added trifluoroacetic acid (1.5 mL) and the resulting solution was stirred at room temperature. After 30 minutes, the reaction was concentrated and the residue was dissolved in ethyl acetate. The solution was treated with anhydrous hydrochloric acid and concentrated. The crude material was loaded onto an SCX ion exchange resin as a solution in acetonitrile/water (1:1) and eluted with ammonia in ethanol solution to give the title compound (80 mg) as a waxy white solid. MS 230 (M+1).

### Step D: N-[(1S,3S)-3-(3-phenyl-1,2,4-oxadiazol-5-yl)cyclopentyl]-1H-pyrazolo[3,4-d]pyrimidin-4-amine

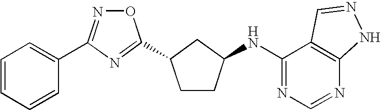

To a solution of [(1S,3S)-3-(3-phenyl-1,2,4-oxadiazol-5-yl)cyclopentyl]amine (40 mg, 0.174 mmol) in isopropanol (3 mL) was added DIPEA (1 mL) and 4-chloro-1-(tetrahydro-pyran-2-yl)-1H-pyrazolo[3,4-d]pyrimidine (44 mg, 0.183 mmol) and the solution was heated at 85° C. for 7 hours. The mixture was cooled and concentrated under reduced pressure. The resulting residue was dissolved in methanol (3 mL) and 6N HCl (0.5 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (18 mg) as a white solid. HRMS (M+1H): calculated=348.1568, observed=348.1569; ¹H NMR (400 MHz, DMSO-d₆): 13.39 (s, 1H), 8.23-8.15 (m, 3H), 8.02 (d, J=6.8 Hz, 2H), 7.58 (d, J=6.8 Hz, 3H), 4.74 (br s, 1H), 3.83-3.79 (m, 1H), 2.40-2.20 (m, 4H), 2.05-2.00 (m, 1H), 1.81 (m, 1H).

The Examples in Table 1 were prepared essentially following the procedures outlined for the preparations of Examples 6 and 7. For examples 9, 11, 12, and 16, commercially available phenyl-nitriles were converted to amidoximes in one step following the procedure of Bakunov, S. A.; Rukavishnikov, A. V.; Tkachev, A. V. Synthesis 8, 1148-1159 (2000).

TABLE 1

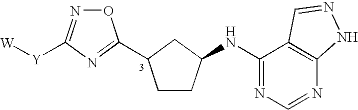

| | | | | HRMS (M + H⁺): |
|---|---|---|---|---|
| Exam- | | | | Observed |
| ples | C-3 | Y | W | [Calculated] |
| 8 | S | — | 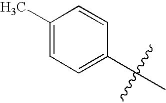 | 362.1701 [362.1724] |
| 9 | S | — | 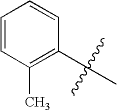 | 362.1728 [362.1724] |
| 10 | S | — | 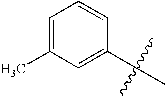 | 362.1729 [362.1724] |
| 11 | S | — | 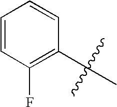 | 366.1459 [366.1473] |
| 12 | S | — | 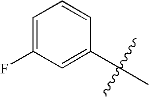 | 362.1474 [366.1473] |
| 13 | S | — | 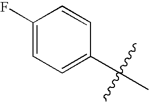 | 362.1475 [366.1473] |
| 14 | S | — | 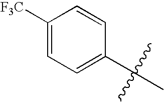 | 416.1457 [416.1441] |
| 15 | S | — | 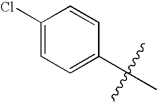 | 382.1161 [382.1178] |
| 16 | S | — | 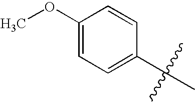 | 378.1627 [378.1673] |
| 17 | S | — | 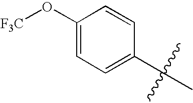 | 432.1372 [432.1391] |

### EXAMPLE 18

### N-[(1S,3S)-3-(3-benzyl-1,2,4-oxadiazol-5-yl)cyclopentyl]-1H-pyrazolo[3,4-d]pyrimidin-4-amine

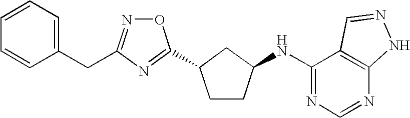

### Step A: (1Z)-N′-hydroxy-2-phenylethanimidamide

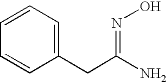

The experimental procedure is based on literature precedent (Bakunov, S. A.; Rukavishnikov, A. V.; Tkachev, A. V. Synthesis 2000, 8, 1148-1159). To a solution of phenylacetonitrile (10.0 g, 85.4 mmol) in 95% ethanol (200 mL) was added hydroxylamine hydrochloride (23.7 g, 341 mmol) and sodium carbonate (36.2 g, 341 mmol), and the resulting solution was heated to 85° C. After 5 hours, the reaction was cooled to room temperature, filtered, and concentrated. The residue was suspended in ether and extracted with 1M hydrochloric acid. The combined aqueous layers were basified with ammonium hydroxide, saturated with sodium chloride, and extracted with ethyl acetate. The organic layer was dried over sodium sulfate, filtered and concentrated to give the title compound (8.45 g) as a waxy solid. HRMS (M+H⁺): calculated=157.0866, observed=157.0862; ¹H NMR (400 MHz, DMSO-d₆): 8.87 (s, 1H), 7.28-7.18 (m, 5H), 5.37 (s, 2H), 3.25 (s, 2H).

### Step B: tert-butyl {(1S,3S)-3-[({[(1Z)-1-amino-2-phenylethylidene]amino}oxy)carbonyl]cyclopentyl}carbamate

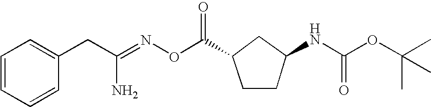

To a solution of (1S,3S)-3-[(tert-butoxycarbonyl)amino]cyclopentanecarboxylic acid (1.99 g, 8.68 mmol) in methylene chloride (20 mL) at room temperature was added HOBt (1.40 g, 9.11 mmol), and EDC (1.83 g, 9.55 mmol). After 40 minutes, (1Z)-N′-hydroxy-2-phenylethanimidamide (1.70 g, 11.3 mmol) was added to the reaction. After 8 hours, the reaction was poured into saturated sodium bicarbonate solution and extracted with methylene chloride. The combined organic layers were washed with 1M sodium hydroxide, dried over sodium sulfate, filtered and concentrated to give the title compound (3.97 g) as a waxy solid. MS 362 (M+1).

### Step C: tert-butyl [(1S,3S)-3-(3-benzyl-1,2,4-oxadiazol-5-yl)cyclopentyl]carbamate

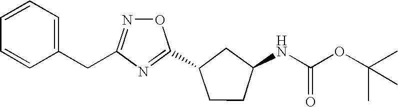

To a solution of tert-butyl {(1S,3S)-3-[({[(1Z)-1-amino-2-phenylethylidene]amino}oxy)carbonyl]cyclopentyl}carbamate (3.97 g, 11.0 mmol) in ethanol (50 mL, 95%) was added sodium carbonate (3.49 g, 33.0 mmol) and the resulting solution was heated to 85° C. After 14 hours, the reaction was cooled to room temperature, partially concentrated, poured into water, and extracted with methylene chloride. The combined organic layers were washed with brine, dried over sodium sulfate, filtered and concentrated. Purification by silica gel chromatography (1% ethyl acetate/hexanes→70% ethyl acetate/hexanes) gave the title compound (1.27 g) as a white solid. HRMS (M+H⁺): calculated=344.1969, observed=344.1975; ¹H NMR (400 MHz, DMSO-d₆): 7.34-7.23 (m, 5H), 7.00 (d, J=6.8 Hz, 1H), 4.05 (s, 2H), 3.93 (m, 1H), 3.56-3.52 (m, 1H), 2.17-1.88 (m, 4H), 1.77-1.72 (m, 1H), 1.55-1.50 (m, 1H), 1.38 (s, 9H).

### Step D: [(1S,3S)-3-(3-benzyl-1,2,4-oxadiazol-5-yl)cyclopentyl]amine

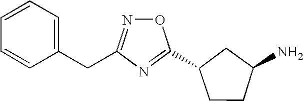

To tert-butyl [(1S,3S)-3-(3-benzyl-1,2,4-oxadiazol-5-yl)cyclopentyl]carbamate (1.10 g, 3.20 mmol) was added trifluoroacetic acid (5.0 mL) and the resulting solution was stirred at room temperature. After 15 minutes, the reaction was concentrated and the residue was dissolved in ethyl acetate. The solution was treated with anhydrous hydrochloric acid and filtered to give the hydrochloride salt of the title compound (882 mg) as a white solid. HRMS (M+H⁺): calculated=244.1445, observed=244.1447; ¹H NMR (400 MHz, CD₃OD): 7.32-7.22 (m, 5H), 4.04 (s, 2H), 3.81-3.78 (m, 1H), 3.68-3.64 (m, 1H), 2.48-2.27 (m, 3H), 2.17-2.10 (m, 1H), 2.03-1.97 (m, 1H), 1.77-1.72 (m, 1H).

### Step E: N-[(1S,3S)-3-(3-benzyl-1,2,4-oxadiazol-5-yl)cyclopentyl]-1H-pyrazolo[3,4-d]pyrimidin-4-amine

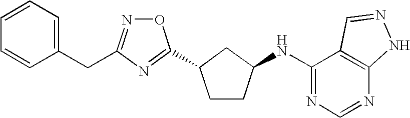

To a solution of [(1S,3S)-3-(3-benzyl-1,2,4-oxadiazol-5-yl)cyclopentyl]amine (350 mg, 1.25 mmol) in 1-butanol (2.0 mL) was added DIPEA (2.0 mL) and 4-chloro-1-(tetrahydro-pyran-2-yl)-1H-pyrazolo[3,4-d]pyrimidine (343 mg, 1.44 mmol) and the solution was heated at 150° C. for 10 minutes under microwave irradiation. The mixture was cooled and concentrated under reduced pressure. The reaction was repeated on the same scale and the resulting residues were combined. The crude material was dissolved in methanol (20 mL) and 6N HCl (2 mL) and was heated at 60° C. for 30 minutes. The reaction was cooled, quenched with saturated sodium bicarbonate and partially concentrated under reduced pressure. The solution was extracted with ethyl acetate, and the organic layer was dried over sodium sulfate, filtered and concentrated to dryness under reduced pressure. Purification by silica gel chromatography (3% isopropanol/methylene chloride→50% isopropanol/methylene chloride) and reverse phase chromatography (Waters XTerra MSC8, 5% acetonitrile/0.1% trifluoroacetic acid/water 95% acetonitrile/0.1% trifluoroacetic acid/water) gave the title compound (570 mg) as a white solid. HRMS (M+H): calculated=362.1724, observed=362.1724; ¹H NMR (400 MHz, CD₃OD): 8.51 (s, 1H), 8.48 (s, 1H), 7.31-7.23 (m, 5H), 4.86 (m, 1H), 4.05 (s, 2H), 3.74-3.70 (m, 1H), 2.55-2.48 (m, 1H), 2.43-2.36 (m, 2H), 2.32-2.08 (m, 1H), 2.08-1.89 (m, 2H).

### EXAMPLE 19

### N-{(1S,3S)-3-[3-(4-methylbenzyl)-1,2,4-oxadiazol-5-yl]cyclopentyl}-1H-pyrazolo[3,4-d]pyrimidin-4-amine

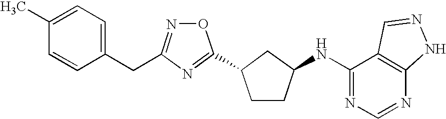

### Step A: (1Z)-N-hydroxy-2-(4-methylphenyl)ethanimidamide

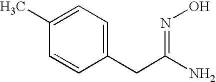

To a solution of (4-methylphenyl)acetonitrile (31.5 g, 240 mmol) in 95% ethanol (525 mL) was added hydroxylamine hydrochloride (67.8 g, 976 mmol) and sodium carbonate (103 g, 976 mmol), and the resulting solution was heated to 85° C. After 14 hours, the reaction was cooled to room temperature, filtered, and concentrated. The residue was suspended in ether and extracted with 1M hydrochloric acid. The combined aqueous layers were basified to pH=9 with ammonium hydroxide, saturated with sodium chloride, and extracted with ethyl acetate. The organic layer was dried over sodium sulfate, filtered and concentrated to give the title compound (30.86 g) as a waxy solid. MS 165.3 (M+1).

### Step B: tert-butyl {(1S,3S)-3-[({[(1Z)-1-amino-2-(4-methylphenyl)ethylidene]amino}oxy)carbonyl]cyclopentyl}carbamate

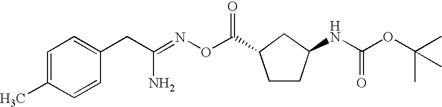

To a solution of (1S,3S)-3-[(tert-butoxycarbonyl)amino]cyclopentanecarboxylic acid (12.9 g, 56.1 mmol) in methylene chloride (150 mL) at room temperature was added HOBt (8.73 g, 57.0 mmol), and EDC (11.0 g, 57.2 mmol). After 20 minutes, (1Z)-N′-hydroxy-2-(4-methylphenyl)ethanimidamide (11.2 g, 68.4 mmol) was added to the reaction and the reaction was permitted to stir an addition 12 hours. The reaction was poured into saturated sodium bicarbonate solution and extracted with ethyl acetate. The combined organic layers were washed with 1M sodium hydroxide, dried over sodium sulfate, filtered and concentrated to give the title compound (19.1 g) as a waxy solid. MS 376.3 (M+1).

### Step C: tert-butyl {(1S,3S)-3-[3-(4-methylbenzyl)-1,2,4-oxadiazol-5-yl]cyclopentyl}carbamate

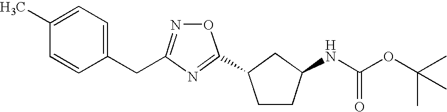

To a solution of tert-butyl {(1S,3S)-3-[({[(1Z)-1-amino-2-(4-methylphenyl)ethylidene]amino}oxy)carbonyl]cyclopentyl}carbamate (19.1 g, 50.9 mmol) in 80% aqueous ethanol (250 mL) was added sodium acetate trihydrate (15.8 g, 116 mmol) and the resulting solution was heated to 85° C. under N₂. After 14 hours, the reaction was cooled to room temperature, partially concentrated, poured into saturated citric acid, and extracted with ethyl acetate. The combined organic layers were washed with saturated sodium bicarbonate, water, brine, dried over sodium sulfate, filtered and concentrated to give the title compound (11.4 g) as a yellow solid. HRMS (M+H⁺): calculated=358.2125, observed=358.2135; ¹H NMR (400 MHz, DMSO-d₆): 7.21-7.09 (m, 4H), 7.00 (d, J=6.4 Hz, 1H), 3.99 (s, 2H), 3.96-3.88 (m, 1H), 3.59-3.48 (m, 1H), 2.27 (s, 3H), 2.20-2.09 (m, 1H), 2.07-1.98 (m, 1H), 1.98-1.85 (m, 2H), 1.79-1.68 (m, 1H), 1.57-1.47 (m, 1H), 1.38 (s, 9H).

### Step D: {(1S,3S)-3-[3-(4-methylbenzyl)-1,2,4-oxadiazol-5-yl]cyclopentyl}amine

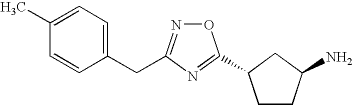

To tert-butyl {(1S,3S)-3-[3-(4-methylbenzyl)-1,2,4-oxadiazol-5-yl]cyclopentyl}carbamate (11.37 g, 6.87 mmol) was added anhydrous hydrochloric acid in ethyl acetate (30 mL) and the resulting solution was stirred at room temperature. After 30 minutes, the reaction was concentrated. The residue was dissolved in 1M hydrochloric acid and washed with ethyl acetate three times. The aqueous layer was basified to pH=8 with ammonium hydroxide, saturated with sodium chloride, and extracted with ethyl acetate. The combined organic layers were dried over sodium sulfate, filtered and concentrated. A solution of the residue in methanol was treated with anhydrous hydrochloric acid in ethyl acetate, and concentrated to yield the hydrochloride salt of title compound (6.87 g) as a white solid. HRMS (M+H⁺): calculated=258.1601, observed=258.1590; ¹H NMR (400 MHz, DMSO-d₆): 8.06 (s, 3H), 7.20-7.09 (m, 4H), 4.00 (s, 2H), 3.74-3.63 (m, 2H), 2.27 (s, 3H), 2.26-2.18 (m, 2H), 2.16-2.06 (m, 2H), 1.87-1.77 (m, 1H), 1.77-1.64 (m, 1H).

### Step E: N-{(1S,3S)-3-[3-(4-methylbenzyl)-1,2,4-oxadiazol-5-yl]cyclopentyl}-1H-pyrazolo[3,4-d]pyrimidin-4-amine

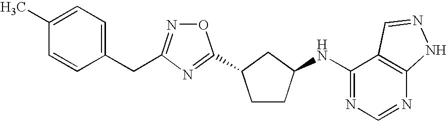

To a solution of {(1S,3S)-3-[3-(4-methylbenzyl)-1,2,4-oxadiazol-5-yl]cyclopentyl}amine (6.87 g, 23.4 mmol) in 1-butanol (50 mL) was added DIPEA (50 mL) and 4-chloro-1-(tetrahydro-pyran-2-yl)-1H-pyrazolo[3,4-d]pyrimidine (5.91 g, 24.8 mmol) and the solution was heated to 90° C. for 3 hours. The mixture was cooled and concentrated under reduced pressure. The resulting residue was dissolved in methanol (15 mL) and a solution of anhydrous hydrochloric acid in ethyl acetate (30 mL) was added. The mixture was stirred for 2 hours, at which time the reaction was quenched with saturated sodium bicarbonate. The solution was extracted with ethyl acetate, and the organic layer was dried over sodium sulfate, filtered and concentrated to dryness under reduced pressure. The compound was recrystallized from ethanol (40 mL) to yield the title compound (4.51 g) as a white solid. HRMS (M+H⁺): calculated=376.1808, observed=376.1881. Elemental Analysis: Calculated for C₂₀H₂₁N₇O: C, 63.98%; H, 5.64%; N, 26.12%. Observed: C, 63.64%; H, 5.42%; N, 25.82%. ¹H NMR (400 MHz, CD₃OD): 8.14 (s, 1H), 8.24 (s, 1H), 7.14-7.20 (m, 4H), 4.77 (m, 1H), 4.00 (s, 2H), 3.73-3.62 (m, 1H), 2.50-2.39 (m, 1H), 2.39-2.32 (m, 2H), 2.32-2.28 (s, 3H), 2.25-2.16 (m, 1H), 2.07-1.95 (m, 1H), 1.90-1.79 (m, 1H).

The Examples in Table 2 were prepared essentially following the procedures outlined for the preparation of Examples 18 and 19. For Example 28, the starting nitrile in Step A is known in the literature (Jin, R.-H.; Andou, Y. Macromolecules 29, 8010-8013 (1996).

TABLE 2

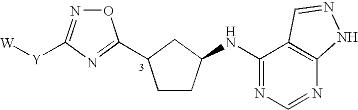

| | | | | HRMS (M + H⁺): |
|---|---|---|---|---|
| | | | | Observed |
| Examples | C-3 | Y | W | [Calculated] |
| 20 | S | CH₂ | 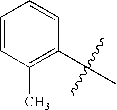 | 376.1901 [376.1881] |
| 21 | S | CH₂ | 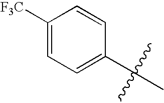 | 430.1600 [430.1598] |
| 22 | S | CH₂ | 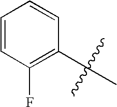 | 380.1621 [380.1630] |
| 23 | S | CH₂ | 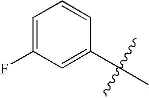 | 380.1631 [380.35] |
| 24 | S | CH₂ | 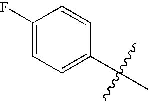 | 380.1620 [380.1630] |
| 25 | S | CH₂ | 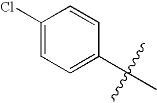 | 396.1357 [396.1334] |
| 26 | S | CH₂ | 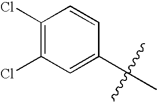 | 430.0946 [430.0945] |
| 27 | S | CH₂ | 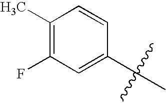 | 394.1786 [394.35] |
| 28 | S | CH₂ | 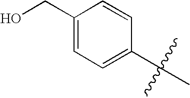 | 392.1836 [392.1830] |
| 29 | S | CH₂ | 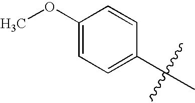 | 392.1820 [392.1830] |
| 30 | S | CH₂ | 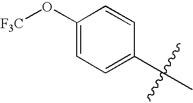 | 446.1534 [446.1547] |
| 31 | S | CH₂ | 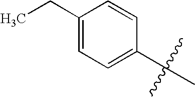 | 390.2042 [390.2037] |
| 32 | S | CH₂ | 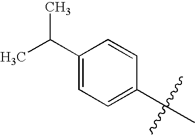 | 404.2182 [404.2194] |
| 33 | S | 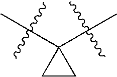 | 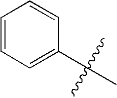 | 388.1918 [388.1881] |
| 34 | S | CH(CH₃) | 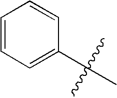 | 376.1919 [376.1881] |
| 35 | S | CH₂CH₂ | 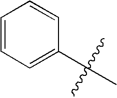 | 376.1884 [376.1881] |

### EXAMPLE 36

### Phenyl{5-[(1S,3S)-3-(1H-pyrazolo[3,4-d]pyrimidin-4-ylamino)cyclopentyl]-1,2,4-oxadiazol-3-yl}methanol

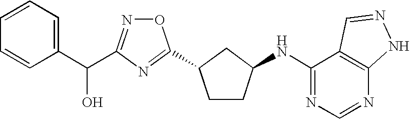

### Step A: Phenyl(tetrahydro-2H-pyran-2-yloxy)acetonitrile

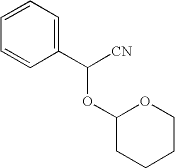

To a solution of hydroxyl(phenyl)acetonitrile (9.84 g, 73.9 mmol) in acetonitrile (200 mL) at 0° C. was added camphorsulfonic acid (0.40 g, 1.7 mmol), followed by the dropwise addition of dihydropyran (8.85 g, 105.2 mmol). The reaction mixture was warmed to room temperature and permitted to react for 24 hrs. The reaction mixture was then quenched by addition of saturated sodium bicarbonate solution and concentrated under reduced pressure. The reaction was diluted with ethyl acetate, washed with water, dried over sodium sulfate, filtered and concentrated to give the title compound (19.4 g) as a waxy solid.

### Step B: (1Z)-N′-Hydroxy-2-phenyl-2-(tetrahydro-2H-pyran-2-yloxy)ethanimidamide

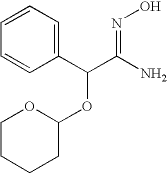

To a solution of phenyl(tetrahydro-2H-pyran-2-yloxy)acetonitrile (10.4 g, 47.9 mmol) in 95% ethanol (260 mL) was added hydroxylamine hydrochloride (13.3 g, 191 mmol) and sodium carbonate (20.3 g, 191 mmol), and the resulting solution was heated to 85° C. overnight. The reaction was cooled to room temperature, filtered, and concentrated. The residue was dissolved in ethyl acetate, washed with water, brine, dried over sodium sulfate, filtered and concentrated to give the title compound (12.0 g) as a waxy solid. LC/MS 251.3 (M+1).

### Step C: tert-Butyl {(1S,3S)-3-[({[(Z)-1-amino-2-hydroxy-2-phenyl-ethylidene]amino}oxy)carbonyl]cyclopentyl}carbamate

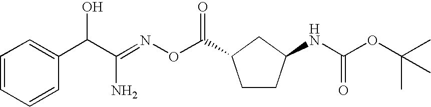

To a solution of (1S,3S)-3-[(tert-butoxycarbonyl)amino]cyclopentanecarboxylic acid (1.0 g, 4.4 mmol) in methylene chloride (50 mL) at room temperature was added HOBt (0.70 g, 4.6 mmol), and EDC (0.92 g, 4.8 mmol). After 20 minutes, (1Z)-N′-hydroxy-2-phenyl-2-(tetrahydro-2H-pyran-2-yloxy)ethanimidamide (1.8 g, 7.4 mmol) was added to the reaction. After 14 hours, the reaction was poured into saturated sodium bicarbonate solution and extracted with ethyl acetate. The combined organic layers were washed with brine, dried over sodium sulfate, filtered and concentrated to give the title compound (0.30 g) as a waxy solid. HRMS (M+H⁺): calculated=378.2024, observed=378.2042.

### Step D: tert-Butyl ((1S,3S)-3-{3-[hydroxy(phenyl)methyl]-1,2,4-oxadiazol-5-yl}cyclopentyl)carbamate

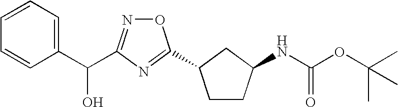

To a solution of tert-butyl {(1S,3S)-3-[({[(1Z)-1-amino-2-hydroxy-2-phenyl-ethylidene]amino}oxy)carbonyl]cyclopentyl}carbamate (0.28 g, 0.80 mmol) in ethanol (50 mL, 80%) was added sodium acetate trihydrate (0.50 g, 3.7 mmol) and the resulting solution was heated to 85° C. while stirring under N₂. After 14 hours, the reaction mixture was cooled to room temperature, partially concentrated under reduced pressure, poured into water, and extracted with ethyl acetate. The combined organic layers were washed with brine, dried over sodium sulfate, filtered and concentrated. HRMS (M+H⁺): calculated=390.1918, observed=390.1910.

### Step E: {5-[(1S,3S)-3-Aminocyclopentyl]-1,2,4-oxadiazol-3-yl}(phenyl)methanol

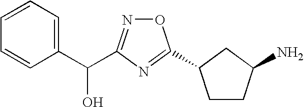

To tert-butyl ((1S,3S)-3-{3-[hydroxy(phenyl)methyl]-1,2,4-oxadiazol-5-yl}cyclopentyl)carbamate (0.14 g, 0.30 mmol) was added trifluoroacetic acid (5.0 mL) and the resulting solution was stirred at room temperature. After 15 minutes, the reaction was concentrated and the residue was dissolved in ethyl acetate. The solution was treated with anhydrous hydrochloric acid and filtered to give the hydrochloride salt of the title compound. This crude material was loaded onto an SCX ion exchange resin as a solution in acetonitrile/water (1:1) and eluted with ammonia in ethanol solution. This solution was then concentrated under reduced pressure, dissolved in methylene chloride, treated with anhydrous hydrochloric acid and concentrated to yield the hydrochloride salt of the title compound (91 mg). HRMS (M+H⁺): calculated=260.1394, observed=260.1377.

### Step F: Phenyl {5-[(1S,3S)-3-(1H-pyrazolo[3,4-d]pyrimidin-4-ylamino)cyclopentyl]-1,2,4-oxadiazol-3-yl}methanol

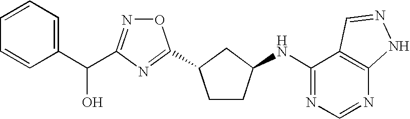

To a solution of {5-[(1S,3S)-3-aminocyclopentyl]-1,2,4-oxadiazol-3-yl}(phenyl)methanol (91 mg, 3.1 mmol) in 1-butanol (1.5 mL) was added DIPEA (1.5 mL) and 4-chloro-1-(tetrahydro-pyran-2-yl)-1H-pyrazolo[3,4-d]pyrimidine (84 mg, 3.5 mmol), and the solution was heated to 100° C. while stirring overnight. The mixture was cooled and concentrated under reduced pressure. The crude material was dissolved in methanol (3 mL) and 6N HCl (1.5 mL) was added at room temperature. The reaction mixture was permitted to stir for 30 minutes. The reaction was then quenched with saturated sodium bicarbonate, extracted with ethyl acetate, and the organic layer was washed with brine, dried over sodium sulfate, filtered and concentrated to dryness under reduced pressure. Purification by reverse phase chromatography (Waters XTerra MSC8, 5% acetonitrile/0.1% trifluoroacetic acid/water→95% acetonitrile/0.1% trifluoroacetic acid/water) gave the title compound (22 mg) as a white solid. LC/MS (M+H⁺): calculated=378.41, observed=378.3; ¹H NMR (400 MHz, CD₃OD): 8.23 (s, 1H), 8.14 (s, 1H), 7.52-7.46 (m, 2H), 7.39-7.27 (m, 3H), 5.89 (s, 1H), 4.76 (s, 1H), 3.74-3.63 (m, 1H), 2.49-2.40 (m, 1H), 2.40-2.29 (m, 2H), 2.26-2.16 (m, 1H), 2.07-1.96 (m, 1H), 1.89-1.78 (m, 1H).

### EXAMPLE 37

### Phenyl{5-[(1S,3S)-3-(1H-pyrazolo[3,4-d]pyrimidin-4-ylamino)cyclopentyl]-1,2,4-oxadiazol-3-yl}methanone

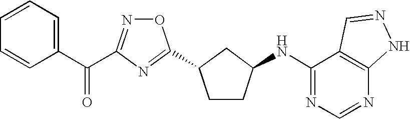

### Step A: tert-butyl [(1S,3S)-3-(3-benzoyl-1,2,4-oxadiazol-5-yl)cyclopentyl]carbamate

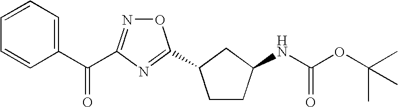

To a solution of tert-butyl ((1S,3S)-3-{3-[hydroxy(phenyl)methyl]-1,2,4-oxadiazol-5-yl}cyclopentyl)carbamate (0.21 g, 0.58 mmol) in methylene chloride (15 mL) was added Dess Martin periodinane (0.28 g, 0.67 mmol) at room temperature. After 3 hours, the reaction was poured into ethyl acetate and washed with 1:1 sat. sodium bicarbonate:sodium thiosulfate, washed with brine, dried over sodium sulfate, filtered and concentrated. Purification by silica gel chromatography (1% isopropanol/methylene chlrodice→6% isopropanol/methylene chloride) gave the title compound (138 mg) as a clear oil. HRMS (M+Na⁺): calculated=380.1581, observed=380.1589.

### Step B: {5-[(1S,3S)-3-aminocyclopentyl]-1,2,4-oxadiazol-3-yl}(phenyl)methanone

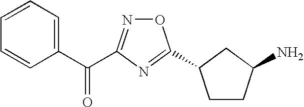

To tert-butyl [(1S,3S)-3-(3-benzoyl-1,2,4-oxadiazol-5-yl)cyclopentyl]carbamate (0.060 g, 0.17 mmol) was added trifluoroacetic acid (5.0 mL) and the resulting solution was stirred at room temperature. After 30 minutes, the reaction was concentrated and the residue was dissolved in ethyl acetate. The solution was treated with anhydrous hydrochloric acid and filtered to give the hydrochloride salt of the title compound. This crude material was loaded onto an SCX ion exchange resin as a solution in acetonitrile/water (1:1) and eluted with ammonia in ethanol solution. This solution was then concentrated under reduced pressure, dissolved in methylene chloride treated with anhydrous hydrochloric acid and concentrated to yield the hydrochloride salt of the title compound (47 mg). HRMS (M+H⁺): calculated=258.1237, observed=258.1238.

### Step C: Phenyl {5-[(1S,3S)-3-(1H-pyrazolo[3,4-d]pyrimidin-4-ylamino)cyclopentyl]-1,2,4-oxadiazol-3-yl}methanone

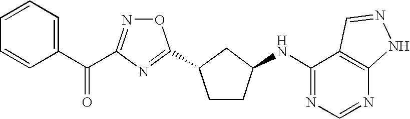

To a solution of {5-[(1S,3S)-3-aminocyclopentyl]-1,2,4-oxadiazol-3-yl}(phenyl)methanone (43 mg, 0.15 mmol) in 1-butanol (2.0 mL) was added DIPEA (2.0 mL) and 4-chloro-1-(tetrahydro-pyran-2-yl)-1H-pyrazolo[3,4-d]pyrimidine (44 mg, 0.18 mmol) and the solution was heated at 90° C. while stirring in a hot oil bath overnight. The mixture was cooled and concentrated under reduced pressure. The crude material was dissolved in methanol (2 mL) and anhydrous hydrochloric acid in ethyl acetate solution (3 mL) was added at room temperature. The reaction was permitted to stir for 30 minutes. The reaction was then quenched with saturated sodium bicarbonate and was extracted with ethyl acetate. The organic layer was washed with brine, dried over sodium sulfate, filtered and concentrated to dryness under reduced pressure. Purification by reverse phase chromatography (Waters XTerra MSC8, 5% acetonitrile/0.1% trifluoroacetic acid/water→95% acetonitrile/0.1% trifluoroacetic acid/water) gave the product, which was dissolved in methanol, treated with anhydrous hydrochloric acid in ethyl acetate solution, and concentrated to yield the hydrochloride salt of the title compound as a mixture of diastereomers (21 mg) HRMS (M+H⁺): calculated=376.1522, observed=376.1518; ¹H NMR (400 MHz, CD₃OD): 8.28-8.21 (m, 3H), 8.15 (s, 1H), 7.76-7.70 (m, 1H), 7.62-7.54 (m, 2H), 3.90-3.81 & 3.80-3.70 (m, 10H), 2.82-2.73 & 2.62-2.23 (m, 4H), 2.22-2.13 (m, 1H), 2.00-1.84 (m, 1H).

### EXAMPLE 38

### N-((1S,3S)-3-{3-[fluoro(phenyl)methyl]-1,2,4-oxadiazol-5-yl}cyclopentyl)-1H-pyrazolo[3,4-d]pyrimidin-4-amine

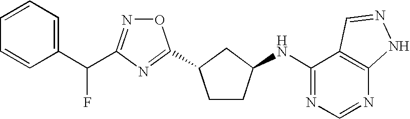

To a solution of phenyl{5-[(1S,3S)-3-(1H-pyrazolo[3,4-d]pyrimidin-4-ylamino)cyclopentyl]-1,2,4-oxadiazol-3-yl}methanol (0.02 g, 0.05 mmol; Example 36) in methylene chloride (4 mL) was added DAST (0.01 g, 0.06 mmol) at 0° C. After 2 hours, the reaction was warmed to room temperature, poured into ice water, and extracted with methylene chloride. The combined organic layers were washed with brine, dried over sodium sulfate, filtered and concentrated. Purification by reverse phase chromatography (Waters XTerra MSC8, 5% acetonitrile/0.1% trifluoroacetic acid/water→95% acetonitrile/0.1% trifluoroacetic acid/water) gave the product, which was dissolved in methanol, treated with anhydrous hydrochloric acid in ethyl acetate solution, and concentrated to yield the hydrochloride salt of the title compound (4.7 mg) as a white solid (mixture of diastereomers at the benzylic position). HRMS (M+H⁺): calculated=380.1630, observed=380.1631; ¹H NMR (400 MHz, CD₃OD): 8.57 (s, 1H), 8.49 (s, 1H), 7.56-7.47 (m, 2H), 7.47-7.37 (m, 3H), 6.71 (s, 0.5H), 6.59 (s, 0.5H), 4.85 (m, 1H (buried beneath H2O peak)), 3.84-3.73 (m, 1H), 2.60-2.49 (m, 1H), 2.49-2.37 (m, 2H), 2.37-2.26 (m, 1H), 2.13-2.01 (m, 1H), 2.01-1.87 (m, 1H).

### EXAMPLE 39

### N-((1S,3S)-3-{3-[difluoro(phenyl)methyl]-1,2,4-oxadiazol-5-yl}cyclopentyl)-1H-pyrazolo[3,4-d]pyrimidin-4-amine

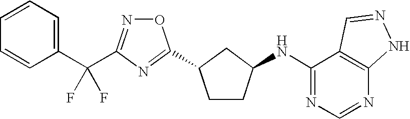

### Step A: 2,2-Difluoro-2-phenylacetamide

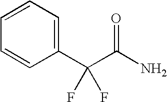

The compound was prepared by the procedures of Sanderson, P. E.; Lyle, T. A.; Dorsey, B. D. WO 9911267 (Mar. 11, 1999) and Middleton, W. J.; Bingham, E. M. J. Org. Chem. 45, 2883-2887 (1980).

Diethylaminosulfur trifluoride (3.82 mL, 31.0 mmol) was added to ethyl oxo(phenyl)acetate (2.46 mL, 15.5 mmol) in one portion and stirred neat at 60° C. After 1 hour the mixture was poured into ice water and extracted into methylene chloride. The organic layer was washed with saturated aqueous sodium bicarbonate and brine, dried over sodium sulfate, filtered and concentrated. The crude residue was dissolved in ethanol (25 mL) and anhydrous ammonia gas was bubbled through the solution for 30 minutes at room temperature at which time the flask was sealed. After 16 hours the solution was concentrated to a solid. The crude product was recrystallized from 7 mL warm EtOAc and 25 mL hot hexanes to afford the title compound (2.05 g) as a solid. LCMS 172 (M+1); ¹H NMR (400 MHz, CDCl₃): 7.63 (d, J=6.86 Hz, 2H), 7.49-7.46 (m, 3H), 6.38 (br s, 1H), 6.01 (br s, 1H).

### Step B: Difluoro(phenyl)acetonitrile

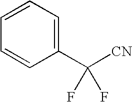

To a solution of 2,2-difluoro-2-phenylacetamide (2.00 g, 11.7 mmol) in THF (50 mL) at 0° C. was added pyridine (2.77 g, 35.1 mmol) and TFAA (2.95 g, 14.0 mmol). After 1 hour, the reaction was warmed to room temperature for 20 minutes, and then the reaction was quenched with water. The mixture was poured into sat. potassium carbonate and extracted with ethyl acetate. The combined organics were washed dried over sodium sulfate, filtered and concentrated to give the title compound (1.00 g) as an oil.

### Step C: (1Z)-2,2-Difluoro-N′-hydroxy-2-phenylethanimidamide

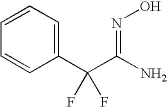

To a solution of difluoro(phenyl)acetonitrile (1.00 g, 6.53 mmol) in 95% ethanol (20 mL) was added hydroxylamine hydrochloride (1.36 g, 19.6 mmol) and sodium carbonate (2.08 g, 19.6 mmol), and the resulting solution was heated to 85° C. After 45 minutes, the reaction was cooled to room temperature, filtered, and concentrated. The residue was suspended in ether and extracted with 1M hydrochloric acid. The combined aqueous layers were basified with ammonium hydroxide, saturated with sodium chloride, and extracted with ethyl acetate. The organic layer was dried over sodium sulfate, filtered and concentrated to give the title compound (149 mg) as a solid. MS 187 (M+1).

### Step D: tert-butyl {(1S,3S)-3-[({[(1Z)-1-amino-2,2-difluoro-2-phenylethylidene]amino}oxy)carbonyl]cyclopentyl}carbamate

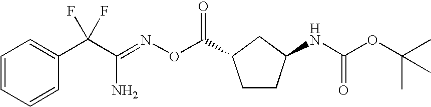

To a solution of (1S,3S)-3-[(tert-butoxycarbonyl)amino]cyclopentanecarboxylic acid (0.17 g, 0.76 mmol) in methylene chloride (20 mL) at room temperature was added HOBt (0.13 g, 0.86 mmol) and EDC (0.16 g, 0.82 mmol). After 20 minutes, (1Z)-2,2-difluoro-N′-hydroxy-2-phenylethanimidamide (0.14 g, 0.75 mmol) was added to the reaction. The reaction was permitted to stir at room temperature overnight. The reaction was poured into saturated sodium bicarbonate solution and extracted with ethyl acetate. The combined organic layers were washed with brine, dried over sodium sulfate, filtered and concentrated to give the title compound (0.274 g) as a solid. HRMS (M+H⁺): calculated=398.1886, observed=398.1886.

### Step E: tert-butyl ((1S,3S)-3-{3-[difluoro(phenyl)methyl]-1,2,4-oxadiazol-5-yl}cyclopentyl)carbamate

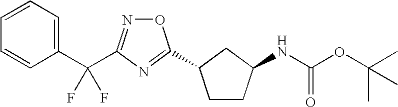

To a solution of tert-butyl {(1S,3S)-3-[({[(1Z)-1-amino-2,2-difluoro-2-phenylethylidene]amino}oxy)carbonyl]cyclopentyl}carbamate (0.26 g, 0.65 mmol) in aqueous ethanol (50 mL, 80%) was added sodium acetate trihydrate (0.31 g, 3.8 mmol) and the resulting solution was heated to 85° overnight while stirring under N₂. The reaction was cooled to room temperature, partially concentrated, poured into water, and extracted with ethyl acetate. The combined organic layers were washed with saturated citric acid, water, brine, dried over sodium sulfate, filtered and concentrated to yield the title compound (235 mg) as an oil. LC/MS (M−55): calculated=324.41, observed=324.3.

### Step F: ((1S,3S)-3-{3-[difluoro(phenyl)methyl]-1,2,4-oxadiazol-5-yl}cyclopentyl)amine

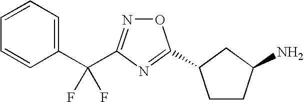

To tert-butyl ((1S,3S)-3-{3-[difluoro(phenyl)methyl]-1,2,4-oxadiazol-5-yl}cyclopentyl)carbamate (0.24 g, 0.62 mmol) was added anhydrous hydrochloric acid in ethyl acetate solution (5.0 mL) and the resulting solution was stirred at room temperature overnight. The reaction was concentrated and the residue was dissolved in ethyl acetate. The solution was treated with anhydrous hydrochloric acid and concentrated to give the hydrochloride salt of the title compound. This crude material was loaded onto an SCX ion exchange resin as a solution in acetonitrile/water (1:1) and eluted with ammonia in ethanol solution. This solution was then concentrated under reduced pressure, dissolved in methylene chloride, treated with anhydrous hydrochloric acid, and concentrated to yield the hydrochloride salt of the title compound (179 mg). HRMS (M+H⁺): calculated=280.1256, observed=280.1259.

### Step G: N-((1S,3S)-3-{3-[difluoro(phenyl)methyl]-1,2,4-oxadiazol-5-yl}cyclopentyl)-1H-pyrazolo[3,4-d]pyrimidin-4-amine

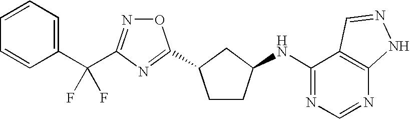

To a solution of ((1S,3S)-3-{3-[difluoro(phenyl)methyl]-1,2,4-oxadiazol-5-yl}cyclopentyl)amine (175 mg, 0.55 mmol) in 1-butanol (2.5 mL) was added DIPEA (2.5 mL) and 4-chloro-1-(tetrahydro-pyran-2-yl)-1H-pyrazolo[3,4-d]pyrimidine (143 mg, 0.60 mmol) and the solution was heated to 90° C. for 3 hours. The mixture was cooled and concentrated under reduced pressure. The crude material was dissolved in methanol (2 mL) and anhydrous hydrochloric acid in ethyl acetate solution (6 mL) was added while stirring at room temperature. After 30 minutes, the reaction mixture was quenched with saturated sodium bicarbonate. The solution was extracted with ethyl acetate, and the organic layer was washed with brine, dried over sodium sulfate, filtered and concentrated to dryness under reduced pressure. Purification by reverse phase chromatography (Waters XTerra MSC8, 5% acetonitrile/0.1% trifluoroacetic acid/water→95% acetonitrile/0.1% trifluoroacetic acid/water) gave the product, which was dissolved in methanol, treated with anhydrous hydrochloric acid in ethyl acetate, and concentrated to yield the hydrochloride salt of the title compound (124 mg) as a solid. HRMS (M+H⁺): calculated=398.1536, observed=398.1542; ¹H NMR (400 MHz, CD₃OD): 8.56 (s, 1H), 8.51 (s, 1H), 7.67-7.61 (m, 2H), 7.56-7.48 (m, 3H), 4.83 (m, 1H (buried beneath H₂O peak)), 3.87-3.78 (m, 1H), 2.62-2.53 (m, 1H), 2.51-2.39 (m, 2H), 2.39-2.29 (m, 1H), 2.16-2.04 (m, 1H), 2.01-1.90 (m, 1H).

### EXAMPLE 40

### (4-Methylphenyl) {5-[(1S,3S)-3-(1H-pyrazolo[3,4-d]pyrimidin-4-ylamino)cyclopentyl]-1,2,4-oxadiazol-3-yl}methanol

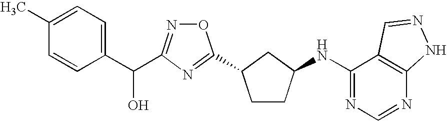

### Step A: Hydroxyl(4-methylphenyl)acetonitrile

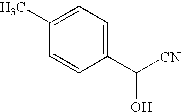

To a solution of 4-methylbenzaldehyde (1.00 g, 8.32 mmol) in methylene chloride (10 mL) was added trimethylsilyl cyanide (1.03 g, 10.4 mmol) and zinc(II) iodide (133 mg, 0.416 mmol) at room temperature. After 14 hours, the reaction was concentrated. The crude residue was dissolved in methanol (50 mL) and treated with 2M HCl (5 mL) at room temperature. After 1 hour, the reaction was concentrated. Purification by silica gel chromatography (1% isopropanol/methylene chloride→25% isopropanol/methylene chloride) gave the title compound (754 mg) as a solid. ¹H NMR (400 MHz, CDCl₃): 7.41 (d, J=8.0 Hz, 2H), 7.25 (d, J=8.0 Hz, 2H), 5.49 (s, 1H), 2.38 (s, 3H).

### Step B: (4-Methylphenyl(tetrahydro-2H-pyran-2-yloxy)acetonitrile

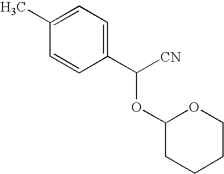

To a solution of hydroxyl(4-methylphenyl)acetonitrile (753 mg, 5.12 mmol) and camphorsulfonic acid (24 mg, 0.10 mmol) in acetonitrile (20 mL) at 0° C. was added dihydropyran (646 mg, 7.67 mmol). The reaction was slowly warmed to room temperature over 2 hours, at which time the reaction was quenched with sat. sodium bicarbonate and partially concentrated. The mixture was diluted with ethyl acetate, washed with sat. sodium bicarbonate, dried over sodium sulfate, filtered and concentrated. Purification by silica gel chromatography (0% isopropanol/methylene chloride→5% isopropanol/methylene chloride) gave the title compound (705 mg) as an oil. ¹H NMR (400 MHz, CD₃OD): 7.42-7.37 (m, 2H), 7.28-7.25 (m, 2H), 5.65 (s, 0.5H), 5.55 (s, 0.5H), 5.05 (m, 0.5H), 4.75 (m, 0.5H), 4.02-4.00 (m, 0.5H), 3.78-3.72 (m, 0.5H), 3.64-3.56 (m, 1H), 2.37 (s, 3H), 1.86-1.50 (m, 6H).

### Step C: (1Z)-N′-Hydroxy-2-(4-methylphenyl)-2-(tetrahydro-2H-pyran-2-yloxy)ethanimidamide

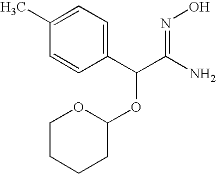

To a solution of (4-methylphenyl)(tetrahydro-2H-pyran-2-yloxy)acetonitrile (418 mg, 1.81 mmol) in 95% ethanol (10 mL) was added hydroxylamine hydrochloride (377 mg, 5.42 mmol) and sodium carbonate (575 mg, 5.42 mmol), and the resulting solution was heated to 85° C. After 2 hours, the reaction was cooled to room temperature, filtered, and concentrated. The residue was dissolved in water, basified with ammonium hydroxide, poured into brine, and extracted with ethyl acetate. The combined organic layers were dried over sodium sulfate, filtered and concentrated to give the title compound (400 mg) as a solid. MS 265 (M+1).

### Step D: tert-Butyl {(1S,3S)-3-[({[(1Z)-1-amino-2-(4-methylphenyl)-2-(tetrahydro-2H-pyran-2-yloxy)ethylidene]amino}oxy)carbonyl]cyclopentyl}carbamate

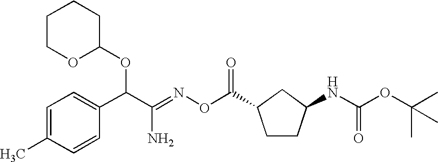

To a solution of (1S,3S)-3-[(tert-butoxycarbonyl)amino]cyclopentanecarboxylic acid (275 mg, 1.20 mmol) in methylene chloride (10 mL) at room temperature was added HOBt (193 mg, 1.26 mmol), and EDC (253 mg, 1.32 mmol). After 15 minutes, (1Z)-N′-hydroxy-2-(4-methylphenyl)-2-(tetrahydro-2H-pyran-2-yloxy)ethanimidamide (412 mg, 1.56 mmol) was added to the reaction. After 1.6 hours, the reaction was poured into saturated sodium bicarbonate solution and extracted with methylene chloride. The combined organic layers were washed with 1M sodium hydroxide, dried over sodium sulfate, filtered and concentrated to give the title compound (570 mg) as a solid. MS 476 (M+1).

### Step E: tert-butyl ((1S,3S)-3-{3-[(4-methylphenyl)(tetrahydro-2H-pyran-2-yloxy)methyl]-1,2,4-oxadiazol-5-yl}cyclopentyl)carbamate

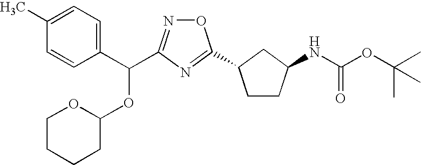

To a solution of tert-butyl {(1S,3S)-3-[({[(1Z)-1-amino-2-(4-methylphenyl)-2-(tetrahydro-2H-pyran-2-yloxy)ethylidene]amino}oxy)carbonyl]cyclopentyl}carbamate (570 mg, 1.20 mmol) in ethanol (10 mL, 95%) was added sodium carbonate (381 mg, 3.60 mmol) and the resulting solution was heated to 85° C. After 14 hours, the reaction was cooled to room temperature, partially concentrated, and poured into ethyl acetate. The organic layer was washed with water, dried over sodium sulfate, filtered and concentrated to give the title compound (600 mg) as an oil. MS 476 (M+1).

### Step F: {5-[(1S,3S)-3-Aminocyclopentyl]-1,2,4-oxadiazol-3-yl}(4-methylphenyl)methanol

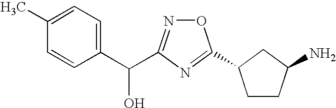

To tert-butyl ((1S,3S)-3-{3-[(4-methylphenyl)(tetrahydro-2H-pyran-2-yloxy)methyl]-1,2,4-oxadiazol-5-yl}cyclopentyl)carbamat (550 mg, 1.20 mmol) was added trifluoroacetic acid (3.0 mL) and the resulting solution was stirred at room temperature. After 30 minutes, the reaction was concentrated and the crude material was loaded onto an SCX ion exchange resin as a solution in acetonitrile/water (1:1) and eluted with ammonia in ethanol solution to give the title compound (323 mg) asanoil.

### Step G: (4-Methylphenyl) {5-[(1S,3S)-3-(1H-pyrazolo[3,4-d]pyrimidin-4-ylamino)cyclopentyl]-1,2,4-oxadiazol-3-yl}methanol

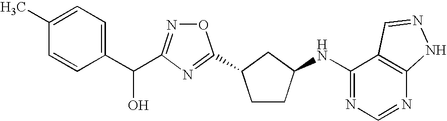

To a solution of {5-[(1S,3S)-3-aminocyclopentyl]-1,2,4-oxadiazol-3-yl}(4-methylphenyl)methanol (570 mg, 1.84 mmol) in 1-butanol (2.0 mL) was added DIPEA (2.0 mL) and 4-chloro-1-(tetrahydro-pyran-2-yl)-1H-pyrazolo[3,4-d]pyrimidine (439 mg, 1.84 mmol) and the solution was heated at 150° C. for 10 minutes under microwave irradiation. The mixture was cooled and concentrated under reduced pressure. The crude material was dissolved in methanol (20 mL) and 6N HCl (2 mL) and was heated at 60° C. for 30 minutes. The reaction was cooled, quenched with saturated sodium bicarbonate and partially concentrated under reduced pressure. The solution was extracted with ethyl acetate, and the organic layer was dried over sodium sulfate, filtered and concentrated to dryness under reduced pressure. Purification by silica gel chromatography (3% isopropanol/methylene chloride→50% isopropanol/methylene chloride) and recrystallization from ethyl acetate gave the title compound (570 mg) as a white solid. HRMS (M+H⁺): calculated=392.1830, observed=392.1836; ¹H NMR (400 MHz, CD₃OD): 8.25 (s, 1H), 8.15 (s, 1H), 7.40-7.15 (m, 4H), 5.85 (s, 1H), 4.8-4.70 (m, 1H), 2.5-2.3 (m, 5H), 2.25-2.15 (m, 1H), 2.05-1.95 (m, 1H), 1.85-1.8 (m, 1H).

### EXAMPLE 41

### N-((1S,3S)-3-{3-[fluoro(4-methylphenyl)methyl]-1,2,4-oxadiazol-5-yl}cyclopentyl)-1H-pyrazolo[3,4-d]pyrimidin-4-amine

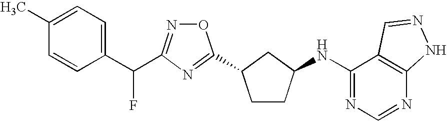

To a solution of (4-methylphenyl){5-[(1S,3S)-3-(1H-pyrazolo[3,4-d]pyrimidin-4-ylamino)cyclopentyl]-1,2,4-oxadiazol-3-yl}methanol (300 mg, 0.766 mmol; Example 36) in methylene chloride (3 mL) was added DAST (0.124 g, 0.766 mmol) at 0° C. After 2 hours, the reaction was cooled to room temperature, partially concentrated, poured into water, and extracted with methylene chloride. The combined organic layers were washed with brine, dried over sodium sulfate, filtered and concentrated. Purification by silica gel chromatography (1% isopropanol/dichloromethane→40% isopropanol/dichloromethane) gave the title compound (100 mg) as a white solid. HRMS (M+H⁺): calculated=394.1786, observed=394.1778; ¹H NMR (400 MHz, CD₃OD): 8.26-8.10 (m, 2H), 7.42-7.18 (m, 4H), 6.66-6.50 (m, 1H), 4.85-4.7 (m, 1H), 3.80-3.60 (m, 1H), 2.5-2.3 (m, 4H), 2.3-2.15 (m, 1H), 2.1-2.0 (m, 1H), 1.9-1.8 (m, 1H).

### EXAMPLE 42

### N-((1S,3S)-3-{3-[difluoro(phenyl)methyl]-1,2,4-oxadiazol-5-yl}cyclopentyl)-1H-pyrazolo[3,4-d]pyrimidin-4-amine

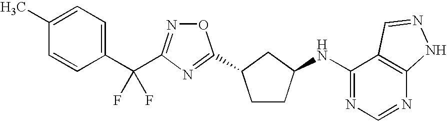

### Step A: 2,2-Difluoro-2-(4-methylphenyl)acetamide

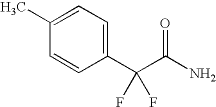

The compound was prepared by modification to the procedure of Sato, K.; Kawata, R.; Ama, F.; Omote, M.; Ando, A. Chemical & Pharmaceutical Bulletin 47(7), 1013-1016 (1999).

To a solution of 1-iodo-4-methylbenzene (25.1 g, 115 mmol) in DMSO (125 mL) was added ethyl bromo(difluoro)acetate (24.7 g, 122 mmol) and copper (16.8 g, 264 mmol), and the resulting solution was heated to 55° C. After 14 hours, the reaction was cooled to room temperature, diluted with isopropyl acetate, cooled to 0° C. and treated with an aqueous solution of potassium hydrogen phosphate (23.3 g in 250 mL water). The mixture was filtered through a pad of Celite and extracted with isopropyl acetate. The combined organics were washed with brine, dried over sodium sulfate, filtered and concentrated. Purification by silica gel chromatography (1% ethyl acetate/5% methylene chloride/hexanes→8% ethyl acetate/5% methylene chloride/hexanes) gave the intermediate ester, which was dissolved in anhydrous 10% ammonia in ethanol (30 mL) and the mixture was heated to 60° C. After 1 hour, the solution was concentrated and the crude material was recrystallized from ethanol to give the title compound as a solid. MS 186 (M+1).

### Step B: Difluoro(4-methylphenyl)acetonitrile

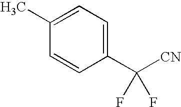

To a solution of 2,2-difluoro-2-(4-methylphenyl)acetamide (10.0 g, 85.4 mmol) in methylene chloride (200 mL) at 0° C. was added triethylamine (23.7 g, 341 mmol) and TFAA (36.2 g, 341 mmol). After 5 hours, the reaction was quenched with sat. sodium bicarbonate, washed with water, dried over sodium sulfate, filtered and concentrated to give the title compound (8.45 g) as a waxy solid. MS 168 (M+1).

### Step C: (1Z)-2,2-Difluoro-N′-hydroxy-2-(4-methylphenyl)ethanimidamide

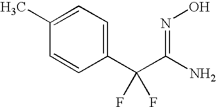

To a solution of difluoro(4-methylphenyl)acetonitrile (5.0 g, 30 mmol) in 95% ethanol (200 mL) was added hydroxylamine hydrochloride (24 g, 340 mmol) and sodium carbonate (36 g, 340 mmol), and the resulting solution was heated to 85° C. After 5 hours, the reaction was cooled to room temperature, filtered, and concentrated. The residue was suspended in ether and extracted with 1M hydrochloric acid. The combined aqueous layers were basified with ammonium hydroxide, saturated with sodium chloride, and extracted with ethyl acetate. The organic layer was dried over sodium sulfate, filtered and concentrated to give the title compound (4.5 g) as a waxy solid. MS 201 (M+1).

### Step D: tert-butyl {(1S,3S)-3-[({[(1Z)-1-amino-2,2-difluoro-2-(4-methylphenyl)ethylidene]amino}oxy)carbonyl]cyclopentyl}carbamate

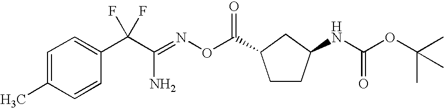

To a solution of (1S,3S)-3-[(tert-butoxycarbonyl)amino]cyclopentanecarboxylic acid (425 mg, 1.85 mmol) in methylene chloride (5 mL) at room temperature was added HOBt (250 mg, 1.85 mmol), and EDC (355 mg, 1.85 mmol). After 40 minutes, (1Z)-2,2-difluoro-N′-hydroxy-2-(4-methylphenyl)ethanimidamide (371 mg, 1.85 mmol) was added to the reaction. After 8 hours, the reaction was poured into saturated sodium bicarbonate solution and extracted with methylene chloride. The combined organic layers were washed with 1M sodium hydroxide, dried over sodium sulfate, filtered and concentrated to give the title compound (600 mg) as a waxy solid. MS 362 (M+1).

### Step E: tert-butyl ((1S,3S)-3-{3-[difluoro(4-methylphenyl)methyl]-1,2,4-oxadiazol-5-yl}cyclopentyl)carbamate

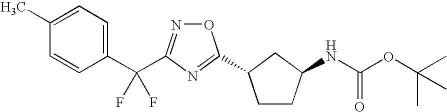

To a solution of tert-butyl {(1S,3S)-3-[({[(1Z)-1-amino-2,2-difluoro-2-(4-methylphenyl)ethylidene]amino}oxy)carbonyl]cyclopentyl}carbamate (500 mg, 1.22 mmol) in ethanol (25 mL, 95%) was added sodium carbonate (3.49 g, 33.0 mmol) and the resulting solution was heated to 85° C. After 14 hours, the reaction was cooled to room temperature, partially concentrated, poured into water, and extracted with methylene chloride. The combined organic layers were washed with brine, dried over sodium sulfate, filtered and concentrated. Purification by silica gel chromatography (1% ethyl acetate/hexanes→70% ethyl acetate/hexanes) gave the title compound (350 mg) as a white solid MS 394 (M+1).

### Step F: ((1S,3S)-3-{3-[difluoro(4-methylphenyl)methyl]-1,2,4-oxadiazol-5-yl}cyclopentyl)amine

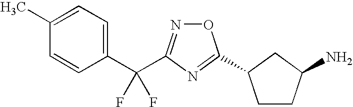

To tert-butyl ((1S,3S)-3-{3-[difluoro(4-methylphenyl)methyl]-1,2,4-oxadiazol-5-yl}cyclopentyl)carbamate (300 mg, 0.763 mmol) was added trifluoroacetic acid (5.0 mL) and the resulting solution was stirred at room temperature. After 15 minutes, the reaction was concentrated and the residue was dissolved in ethyl acetate. The solution was treated with anhydrous hydrochloric acid and filtered to give the hydrochloride salt of the title compound (258 mg) as a white solid. MS 294 (M+1).

### Step G: N-((1S,3S)-3-{3-[difluoro(4-methylphenyl)methyl]-1,2,4-oxadiazol-5-yl}cyclopentyl)-1H-pyrazolo[3,4-d]pyrimidin-4-amine

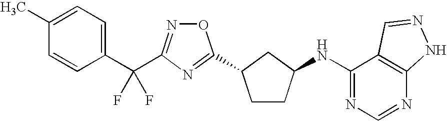

To a solution of ((1S,3S)-3-{3-[difluoro(phenyl)methyl]-1,2,4-oxadiazol-5-yl}cyclopentyl)amine (350 mg, 1.06 mmol) in 1-butanol (2.0 mL) was added DIPEA (2.0 mL) and 4-chloro-1-(tetrahydro-pyran-2-yl)-1H-pyrazolo[3,4-d]pyrimidine (253 mg, 1.06 mmol) and the solution was heated at 150° C. for 10 minutes under microwave irradiation. The mixture was cooled and concentrated under reduced pressure. The crude material was dissolved in methanol (2 mL) and anhydrous hydrochloric acid in ethyl acetate (5 ml) was added. After 1 hour, the reaction was concentrated under reduced pressure, dissolved in ethyl acetate, washed with sodium bicarbonate, dried over sodium sulfate, filtered and concentrated to dryness under reduced pressure. Purification by silica gel chromatography (3% isopropanol/methylene chloride→50% isopropanol/methylene chloride) and recrystallization from ethyl acetate and methanol gave the title compound (250 mg) as a white solid. HRMS (M+H⁺): calculated=412.1692, observed=412.1696; ¹H NMR (400 MHz, CD₃OD): 8.24 (s, 1H), 8.13 (s, 1H), 7.52-7.48 (m, 2H), 7.34-7.30 (m, 2H), 4.80-4.7 (m, 1H), 2.5-2.3 (m, 6H), 2.3-2.2 (m, 1H), 2.1-2.0 (m, 1H), 1.90-1.80 (m, 1H).

### EXAMPLE 43

### N-[(1S,3S)-3-(5-phenyl-1,2,4-oxadiazol-3-yl)cyclopentyl]-1H-pyrazolo[3,4-d]pyrimidin-4-amine

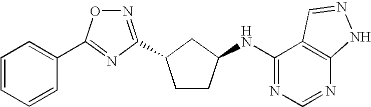

### Step A: tert-butyl [(1S,3S)-3-cyanocyclopentyl]carbamate

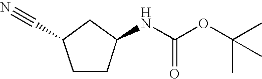

To a solution of tert-butyl [(1S,3S)-3-(aminocarbonyl)cyclopentyl]carbamate (200 mg, 0.876 mmol; from Example 5, Step A) in tetrahydrofuran (15 mL) at 0° C. was added pyridine (139 mg, 1.75 mmol) and trifluoroacetic anhydride (221 mg, 1.05 mmol). The reaction was slowly warmed to room temperature over a 2 hour period. The reaction was quenched with water, poured into saturated potassium carbonate, and extracted with ethyl acetate. The organic layer was washed with brine, dried over sodium sulfate, filtered and concentrated to give the title compound (230 mg) as a white solid. This material was sufficiently pure for the next step. ¹H NMR (400 MHz, CDCl₃): 4.43 (br s, 1H), 4.15-4.10 (m, 1H), 2.96-2.88 (m, 1H), 2.34 (m, 1H), 2.27-2.13 (m, 3H), 2.02-1.88 (m, 2H), 1.44 (s, 9H).

### Step B: tert-butyl {(1S,3S)-3-[(Z)-amino(hydroxyimino)methyl]cyclopentyl}carbamate

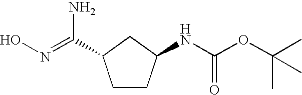

To a solution of tert-butyl [(1S,3S)-3-cyanocyclopentyl]carbamate (220 mg, 1.07 mmol) in 95% ethanol (10 mL) was added hydroxylamine hydrochloride (95 mg, 1.36 mmol) and sodium carbonate (144 mg, 1.36 mmol), and the resulting solution was heated to 85° C. After 3 hours, another batch of hydroxylamine hydrochloride (95 mg, 1.36 mmol) and sodium carbonate (144 mg, 1.36 mmol) was added, and the resulting solution was maintained at 85° C. After 72 hours, the reaction was cooled to room temperature, filtered, and concentrated. The residue was suspended in ether and extracted with 1M hydrochloric acid. The combined aqueous layers were basified with ammonium hydroxide, saturated with sodium chloride, and extracted with ethyl acetate. The organic layer was dried over sodium sulfate, filtered and concentrated to give the title compound (191 mg) as a white solid. This material was sufficiently pure for the next step. MS 244 (M+1); ¹H NMR (400 MHz, DMSO-d₆): 6.80 (s, 1H), 5.22 (s, 2H), 3.83 (m, 1H), 2.62-2.57 (m, 1H), 1.90-1.75 (m, 4H), 1.61-1.56 (m, 2H), 1.37 (s, 9H).

### Step C: tert-butyl ((1S,3S)-3-{(Z)-amino[(benzoyloxy)imino]methyl}cyclopentyl)carbamate

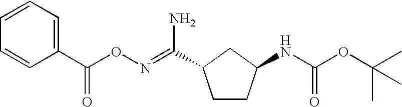

To a solution of tert-butyl {(1S,3S)-3-[(Z)-amino(hydroxyimino)methyl]cyclopentyl}carbamate (100 mg, 0.411 mmol) in methylene chloride (10 mL) at room temperature was added DIPEA (127 mg, 0.986 mmol) and benzoyl chloride (61 mg, 0.432). After 15 hours, the reaction was quenched with saturated potassium carbonate and extracted with methylene chloride. The organic layer was dried over sodium sulfate, filtered and concentrated to give the title compound (128 mg) as a white solid. This material was sufficiently pure for the next step.

### Step D: tert-butyl [(1S,3S)-3-(5-phenyl-1,2,4-oxadiazol-3-yl)cyclopentyl]carbamate

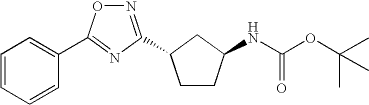

To a solution of tert-butyl {(1S,3S)-3-[(Z)-amino(hydroxyimino)methyl]cyclopentyl}carbamate (128 mg, 0.368 mmol) in ethanol (5 mL) and water (1.0 ml) was added sodium acetate (60 mg, 0.737 mmol) and the resulting solution was heated to 85° C. After 15 hours, the reaction was cooled to room temperature, concentrated and dissolved in ethyl acetate. The organic layer was washed with water, saturated potassium carbonate, dried over sodium sulfate, filtered and concentrated. Purification by silica gel chromatography (10% ethyl acetate/5% methylene chloride/hexanes→50% ethyl acetate/5% methylene chloride/hexanes) gave the title compound (74 mg) as a white solid. MS 330 (M+1) and 274 (M−55).

### Step E: [(1S,3S)-3-(5-phenyl-1,2,4-oxadiazol-3-yl)cyclopentyl]amine

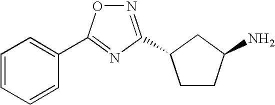

To tert-butyl [(1S,3S)-3-(5-phenyl-1,2,4-oxadiazol-3-yl)cyclopentyl]carbamate (74 mg, 0.225 mmol) was added trifluoroacetic acid (1.5 mL) and the resulting solution was stirred at room temperature. After 1 hour, the reaction was concentrated and the residue was dissolved in ethyl acetate. The solution was treated with anhydrous hydrochloric acid and filtered to give the hydrochloride salt of the title compound (80 mg). MS 230 (M+1).

### Step F: N-[(1S,3S)-3-(5-phenyl-1,2,4-oxadiazol-3-yl)cyclopentyl]-1H-pyrazolo[3,4-d]pyrimidin-4-amine

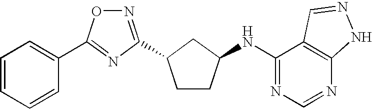

To a solution of [(1S,3S)-3-(5-phenyl-1,2,4-oxadiazol-3-yl)cyclopentyl]amine (60 mg, 0.23 mmol) in 1-butanol (1.1 mL) was added DIPEA (1.1 mL) and 4-chloro-1-(tetrahydro-pyran-2-yl)-1H-pyrazolo[3,4-d]pyrimidine (70 mg, 0.29 mmol) and the solution was heated at 150° C. for 10 minutes under microwave irradiation. The mixture was cooled and concentrated under reduced pressure. The resulting residue was dissolved in methanol (2 mL) and 6N HCl (0.5 mL) and was stirred at room temperature for 1 hour, at which time the reaction was quenched with saturated sodium bicarbonate. The solution was extracted with ethyl acetate, and the organic layer was dried over sodium sulfate, filtered and concentrated to dryness under reduced pressure. Purification by silica gel chromatography (1%/isopropanol/methylene chloride→20% isopropanol/methylene chloride) gave the title compound (54 mg) as a white solid. HRMS (M+H⁺): calculated=348.1568, observed=348.1553; ¹H NMR (400 MHz, DMSO-d₆): 13.38 (s, 1H), 8.23-8.10 (m, 5H), 7.74-7.62 (m, 3H), 4.75 (br s, 1H), 3.65-3.61 (m, 1H), 2.31-2.27 (m, 3H), 2.16-2.11 (m, 1H), 1.96-1.91 (m, 1H), 1.78 (m, 1H).

### EXAMPLE 44

### N-{(1S,3S)-3-[5-(4-methylbenzyl)-1,2,4-oxadiazol-3-yl]cyclopentyl}-1H-pyrazolo[3,4-d]pyrimidin-4-amine

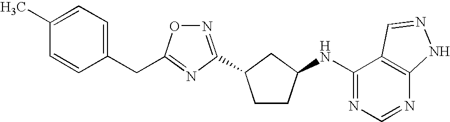

### Step A: tert-butyl {((1S,3S)-3-[(Z)-amino({[(4-methylphenyl)acetyl]oxy}imino)methyl]cyclopentyl}carbamate

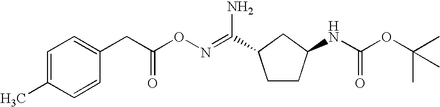

To a solution of [4-(methyl)phenyl]acetic acid (62 mg, 0.41 mmol) in DMF (1.5 mL) at room temperature was added O-benzotriazol-1-yl-N,N,N¹,N¹, tetramethyluronium hexafluorophosphate (132 mg, 0.411 mmol), DIPEA (266 mg, 2.06 mmol) and HOBt (11 mg, 0.082 mmol). After 5 minutes, tert-butyl {(1S,3S)-3-[(Z)-amino(hydroxyimino)methyl]cyclopentyl}carbamate (100 mg, 0.411 mmol) was added to the reaction. After 1.5 hours, the reaction was poured into ethyl acetate. The organic layer was washed with water, brine, dried over sodium sulfate, filtered and concentrated to give the title compound (132 mg) as a yellow solid. This material was sufficiently pure for the next step. MS 376 (M+1).

### Step B: tert-butyl {(1S,3S)-3-[5-(4-methylbenzyl)-1,2,4-oxadiazol-3-yl]cyclopentyl}carbamate

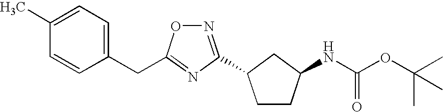

To a solution of tert-butyl {(1S,3S)-3-[(Z)-amino({[(4-methylphenyl)acetyl]oxy}imino)methyl]cyclopentyl}carbamate (132 mg, 0.352 mmol) in ethanol (5 mL) and water (1.0 mL) was added sodium acetate (87 mg, 1.1 mmol) and the resulting solution was heated to 85° C. After 20 hours, the reaction was cooled to room temperature, concentrated and dissolved in ethyl acetate. The organic layer was washed with water, saturated potassium carbonate, dried over sodium sulfate, filtered and concentrated. Purification by silica gel chromatography (1% isopropanol/hexanes→9% isopropanol/hexanes) gave the title compound (68 mg) as a colorless oil. MS 358 (M+1) and 302 (M−55).

### Step C: {(1S,3S)-3-[5-(4-methylbenzyl)-1,2,4-oxadiazol-3-yl]cyclopentyl}amine

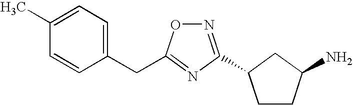

To tert-butyl {(1S,3S)-3-[5-(4-methylbenzyl)-1,2,4-oxadiazol-3-yl]cyclopentyl}carbamate (68 mg, 0.19 mmol) was added trifluoroacetic acid (1.0 mL) and the resulting solution was stirred at room temperature. After 20 minutes, the reaction was concentrated and the residue was dissolved in ethyl acetate. The solution was treated with anhydrous hydrochloric acid and filtered to give the title compound (54 mg). HRMS (M+H⁺): calculated=258.1601, observed=258.1592; ¹H NMR (400 MHz, CD₃OD): 7.19 (d, J=8.0 Hz, 2H), 7.15 (d, J=8.0 Hz, 2H), 4.20 (s, 2H), 3.81-3.78 (m, 1H), 3.53-3.49 (m, 1H), 2.40-2.24 (m, 6H), 2.09-2.02 (m, 1H), 1.95-1.90 (m, 1H), 1.77-1.70 (m, 1H).

### Step D: N-{(1S,3S)-3-[5-(4-methylbenzyl)-1,2,4-oxadiazol-3-yl]cyclopentyl}-1H-pyrazolo[3,4-d]pyrimidin-4-amine

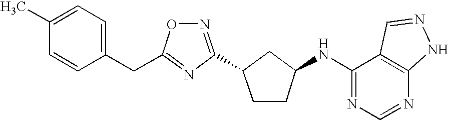

To a solution of {(1S,3S)-3-[5-(4-methylbenzyl)-1,2,4-oxadiazol-3-yl]cyclopentyl}amine (54 mg, 0.19 mmol) in 1-butanol (1.0 mL) was added DIPEA (1.0 mL) and 4-chloro-1-(tetrahydro-pyran-2-yl)-1H-pyrazolo[3,4-d]pyrimidine (48 mg, 0.20 mmol) and the solution was heated at 120° C. for 3 hours. The mixture was cooled and concentrated under reduced pressure. The resulting residue was dissolved in methanol (3 mL) and 6N HCl (0.5 mL) was added at room temperature. After 2 hours, the reaction was quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→35% isopropanol/methylene chloride) gave the title compound, which was dissolved in ethyl acetate and treated with anhydrous hydrochloric acid and filtered to give the hydrochloride salt of the title compound (53 mg) as a white solid. HRMS (M+H⁺): calculated=376.1881, observed=376.1862; ¹H NMR (400 MHz, CD₃OD): 8.50-8.46 (m, 2H), 7.21 (d, J=8.0 Hz, 2H), 7.16 (d, J=8.0 Hz, 2H), 4.85 (m, 1H), 4.22 (s, 2H), 3.59-3.55 (m, 1H), 2.46-2.36 (m, 3H), 2.32 (s, 3H), 2.21 (m, 1H), 2.02-1.89 (m, 2H).

### EXAMPLE 45

### N-((1S,3S)-3-{5-[4-(trifluoromethyl)benzyl]-1,2,4-oxadiazol-3-yl}cyclopentyl)-1H-pyrazolo[3,4-d]pyrimidin-4-amine

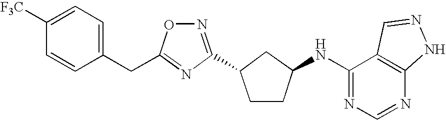

From tert-butyl {(1S,3S)-3-[(Z)-amino(hydroxyimino)methyl]cyclopentyl}carbamate and [4-(trifluoromethyl)phenyl]acetic acid using the procedure described above for Example 44 gave the product as a white crystalline solid: HRMS (M+H): calculated=430.1598, observed=430.1599; ¹H NMR (400 MHz, CD₃OD): 8.26-8.18 (m, 2H), 7.68-7.56 (m, 4H), 4.75 (m, 1H), 4.40 (s, 2H), 3.55 (m, 1H), 2.40-2.30 (m, 3H), 2.15 (m, 1H), 2.0 (m, 1H), 1.80 (m, 1H).

### EXAMPLE 46

### N-{(1S,3S)-3-[5-(4-chlorobenzyl)-1,2,4-oxadiazol-3-yl]cyclopentyl}-1H-pyrazolo[3,4-d]pyrimidin-4-amine

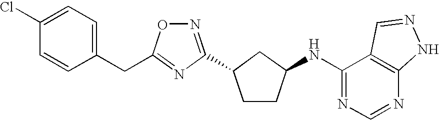

From tert-butyl {(1S,3S)-3-[(Z)-amino(hydroxyimino)methyl]cyclopentyl}carbamate and (4-chlorophenyl)acetic acid using the procedure described above for Example 44 gave the product as a white crystalline solid: HRMS (M+H⁺): calculated=396.1334, observed=396.1335; ¹H NMR (400 MHz, CD₃OD): 8.58 (s, 1H), 8.48 (s, 1H), 7.38-7.32 (m, 4H), 4.30-4.25 (m, 2H), 3.63-3.55 (m, 1H), 2.50-2.30 (m, 3H), 2.28-2.20 (m, 1H), 2.05-1.85 (m, 2H).

### EXAMPLE 47

### N-[(1S,3S)-3-(5-benzyl-1,3,4-oxadiazol-2-yl)cyclopentyl]-1H-pyrazolo[3,4-d]pyrimidin-4-amine

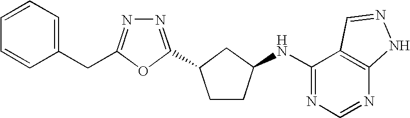

### Step A: tert-butyl ((1S,3S)-3-{[2-(phenylacetyl)hydrazino]carbonyl}cyclopentyl)carbamate

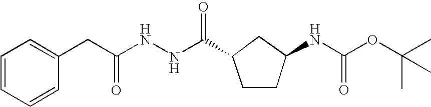

To a solution of (1S,3S)-3-[(tert-butoxycarbonyl)amino]cyclopentanecarboxylic acid (0.83 g, 3.6 mmol) in methylene chloride (15 mL) was added O-benzotriazol-1-yl-N,N,N¹,N¹, tetramethyluronium hexafluorophosphate (2.6 g, 5.9 mmol) at room temperature under N₂. After 30 minutes, 2-phenylacetohydrazide (0.55 g, 3.7 mmol) was added and the reaction was stirred for 14 hours. The gel-like mixture was poured into ethyl acetate and the organic layer was washed with 1 N sodium hydroxide, water, brine, dried over sodium sulfate, filtered and concentrated. Purification by silica gel chromatography (1% isopropanol/methylene chloride→15% isopropanol/methylene chloride) gave the title compound (0.89 g) as a brown solid. This material was sufficiently pure for the next step. HRMS (M+H⁺): calculated=362.2075, observed=362.2073.

### Step B: tert-butyl [(1S,3S)-3-(5-benzyl-1,3,4-oxadiazol-2-yl)cyclopentyl]carbamate

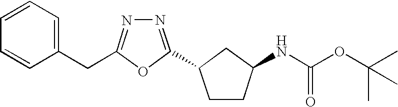

A mixture of tert-butyl ((1S,3S)-3-{[2-(phenylacetyl)hydrazino]carbonyl}cyclopentyl)carbamate (0.20 g, 0.56 mmol) and Burgess reagent (0.56 g, 2.4 mmol) in anhydrous tetrahydrofuran (4 mL) was heated in a sealed tube at 120° C. for 10 minutes under microwave irradiation. The reaction was cooled to room temperature and concentrated under reduced pressure. Purification by reverse phase chromatography (Xterra MS C8; 5-95% acetonitrile/0.1% trifluoroacetic acid/water) yielded the title compound (0.06 g) as a white solid. HRMS (M+H⁺): calculated=344.1969, observed=344.1959.

### Step C: [(1S,3S)-3-(5-benzyl-1,3,4-oxadiazol-2-yl)cyclopentyl]amine

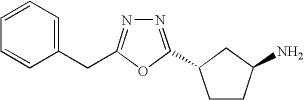

To tert-butyl [(1S,3S)-3-(5-benzyl-1,3,4-oxadiazol-2-yl)cyclopentyl]carbamate (0.060 g 0.16 mmol) was added anhydrous hydrochloric acid in ethyl acetate (6 mL) and the resulting solution was stirred at room temperature. After 20 minutes, the reaction was concentrated. Purification by reverse phase chromatography (Xterra MS C8; 5-95% acetonitrile/0.1% trifluoroacetic acid/water) yielded the title compound which was dissolved in methanol, treated with anhydrous hydrochloric acid in ethyl acetate, and concentrated to yield the hydrochloride salt of the title compound (0.04 g) as a white solid. HRMS (M+H⁺): calculated=244.1445, observed=244.1436.

### Step D: N-[(1S,3S)-3-(5-benzyl-1,3,4-oxadiazol-2-yl)cyclopentyl]-1H-pyrazolo[3,4-d]pyrimidin-4-amine

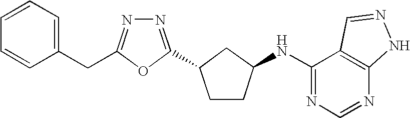

To a solution of [(1S,3S)-3-(5-benzyl-1,3,4-oxadiazol-2-yl)cyclopentyl]amine (0.040 g 0.16 mmol) in 1-butanol (1.5 mL) was added DIPEA (1.5 mL) and 4-chloro-1-(tetrahydro-pyran-2-yl)-1H-pyrazolo[3,4-d]pyrimidine (0.050 g, 0.20 mmol) and the solution was heated at 150° C. for 10 minutes under microwave irradiation. The mixture was cooled and concentrated under reduced pressure. The resulting residue was dissolved in methanol (2 mL) and anhydrous hydrochloric acid in ethyl acetate (3 mL) was added. After 1 hour, the reaction was quenched with saturated sodium bicarbonate. The solution was extracted with ethyl acetate, and the organic layer was dried over sodium sulfate, filtered and concentrated to dryness under reduced pressure. Purification by reverse phase chromatography (Xterra MS C8; 5-95% acetonitrile/0.1% trifluoroacetic acid/water) yielded the title compound which was dissolved in methanol, treated with anhydrous hydrochloric acid in ethyl acetate, and concentrated to yield the hydrochloride salt of the title compound (0.01 g) as a white solid. HRMS (M+H⁺): calculated=380.1380, observed=380.1825; ¹H NMR (400 MHz, CD₃OD): 8.53 (s, 1H), 8.46 (s, 1H), 7.38-7.19 (m, 5H), 4.80-4.59 (m, 1H), 3.58 (s, 2H), 3.11-3.00 (s, 1H), 2.50-2.39 (m, 1H), 2.39-2.27 (m, 1H), 2.26-2.14 (m, 1H), 2.06-1.91 (m, 2H), 2.23 (m, 2H), 1.88-1.77 (m, 1H).

### EXAMPLE 48

### N-{(1S,3S) and (1S,3R)-3-[5-(4-methylbenzyl)-1,3,4-thiadiazol-2-yl]cyclopentyl}-1H-pyrazolo[3,4-d]pyrimidin-4-amine

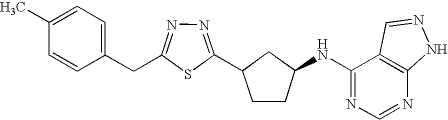

### Step A: 2-(4-methylphenyl)acetohydrazide

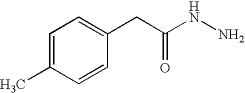

To a solution of (4-methylphenyl)acetic acid (2.05 g, 13.6 mmol) in anhydrous acetonitrile (50 mL) at room temperature was added EDC (4.52 g, 23.6 mmol). After 30 minutes, 98% anhydrous hydrazine (1.49 mL, 47.3 mmol) was added to the reaction. The reaction was permitted to stir overnight under N₂, at which point the reaction was poured into ethyl acetate. The organic layer was washed with water, brine, dried over sodium sulfate, filtered and concentrated. Purification by silica gel chromatography (10% isopropanol/methylene chloride→60% isopropanol/methylene chloride) gave the title compound (0.90 g) as a white solid. HRMS (M+H⁺): calculated=165.1072, observed=165.1067.

### Step B: tert-butyl [(1S,3S)-3-({2-[(4-methylphenyl)acetyl]hydrazino}carbonyl)cyclopentyl]carbamate

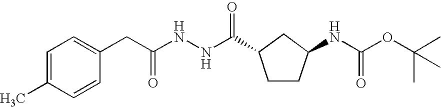

A mixture of (1S,3S)-3-[(tert-butoxycarbonyl)amino]cyclopentanecarboxylic acid (1.10 g, 4.80 mmol), HOBt (0.78 g, 5.07 mmol), and EDC (0.97 g, 5.07 mmol) in methylene chloride (220 mL) and DMF (25 mL) was stirred at room temperature under N₂. After 30 minutes, 2-(4-methylphenyl)acetohydrazide (0.80 g, 4.87 mmol) was added to the reaction. After 14 hours, the gel-like mixture was poured into ethyl acetate. The organic layer was washed with 1 N sodium hydroxide, water, brine, dried over sodium sulfate, filtered and concentrated to yield the title compound (1.82 g) as a white solid. HRMS (M+Na⁺): calculated=398.2050, observed=398.2059.

### Step C: {(1S,3S) and (1S,3R)-3-[5-(4-methylbenzyl)-1,3,4-thiadiazol-2-yl]cyclopentyl}amine

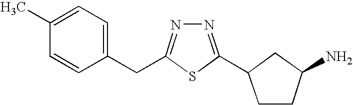

The reaction mixture was prepared by addition of tert-butyl [(1S,3S)-3-({2-[(4-methylphenyl)acetyl]hydrazino}carbonyl)cyclopentyl]carbamate (0.50 g, 1.3 mmol) and Lawesson's reagent (1.2 g, 2.9 mmol) in anhydrous toluene (5 mL). The reaction mixture was heated to 150° C. for 10 minutes under microwave irradiation. The reaction was cooled to room temperature, concentrated and dissolved in ethyl acetate. The organic layer was washed with 1M sodium hydroxide, water, brine, dried over sodium sulfate, filtered and concentrated. Then trifluoroacetic acid (5.0 mL) was added and the resulting solution was stirred at room temperature. After 20 minutes, the reaction was concentrated and the residue was dissolved in ethyl acetate. The solution was treated with anhydrous hydrochloric acid and concentrated. Purification by reverse phase chromatography (Xterra MS C8; 5-95% acetonitrile/0.1% trifluoroacetic acid/water) gave the title compound which was dissolved in methanol, treated with anhydrous hydrochloric acid in ethyl acetate, and concentrated to yield the hydrochloride salt of the title compound (0.24 g) as a tan solid. HRMS (M+H⁺): calculated=274.1313, observed=274.1365.

### Step D: N-{(1S,3S) and (1S,3R)-3-[5-(4-methylbenzyl)-1,3,4-thiadiazol-2-yl]cyclopentyl}-1H-pyrazolo[3,4-d]pyrimidin-4-amine

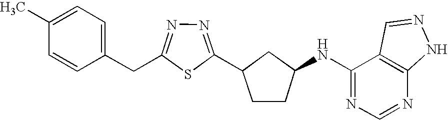

To a solution of {(1S,3S) and (1S,3R)-3-[5-(4-methylbenzyl)-1,3,4-thiadiazol-2-yl]cyclopentyl}amine (0.15 g, 0.50 mmol) in 1-butanol (1.5 mL) was added DIPEA (1.5 mL) and 4-chloro-1-(tetrahydro-pyran-2-yl)-1H-pyrazolo[3,4-d]pyrimidine (0.14 g, 0.58 mmol) and the solution was heated at 150° C. for 10 minutes under microwave irradiation. The mixture was cooled and concentrated under reduced pressure. The resulting residue was dissolved in methanol (3 mL) and anhydrous hydrochloric acid in ethyl acetate (5 mL) was added. The reaction was stirred for 1 hour, at which time the reaction was quenched with saturated sodium bicarbonate. The solution was extracted with ethyl acetate, and the organic layer was dried over sodium sulfate, filtered and concentrated to dryness under reduced pressure. Purification by reverse phase chromatography (ChiralPak AD; 60% isopropanol/40% hexane) gave the title compounds which were dissolved in methanol, treated with anhydrous hydrochloric acid in ethyl acetate, and concentrated to yield the hydrochloride salt of the title compounds.

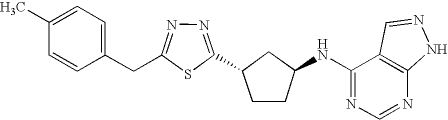

### N-{(1S,3S)-3-[5-(4-methylbenzyl)-1,3,4-thiadiazol-2-yl]cyclopentyl}-1H-pyrazolo[3,4-d]pyrimidin-4-amine: HRMS (M+H⁺): calculated=392.1692, observed=392.1693. ¹H NMR (400 MHz, CD₃OD): 8.52 (s, 1H), 8.48 (s, 1H), 7.23-7.13 (m, 4H), 4.82-4.78 (m, 1H), 4.36 (s, 2H), 3.92-3.82 (m, 1H), 2.52-2.38 (m, 3H), 2.37-2.32 (m, 1H), 2.31 (s, 3H), 2.03-1.86 (m, 2H).

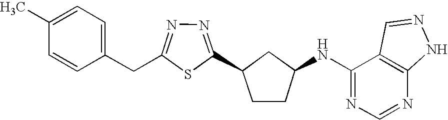

### N-{(1S,3R)-3-[5-(4-methylbenzyl)-1,3,4-thiadiazol-2-yl]cyclopentyl)}-1H-pyrazolo[3,4-d]pyrimidin-4-amine: HRMS (M+H⁺): calculated=392.1692, observed=392.1659. ¹H NMR (400 MHz, CD₃OD): 8.52 (s, 1H), 8.50 (s, 1H), 7.22-7.12 (m, 4H), 4.82-4.77 (m, 1H), 4.36 (s, 2H), 3.80-3.70 (m, 1H), 2.82-2.73 (m, 1H), 2.40-2.32 (m, 2H), 2.31 (s, 3H), 2.14-2.03 (m, 2H), 2.03-1.93 (m, 1H).

### EXAMPLE 49

### N-[(1S,3R)-3-(5-benzyl-4-methyl-4H-1,2,4-triazol-3-yl)cyclopentyl]-1H-pyrazolo[3,4-d]pyrimidin-4-amine

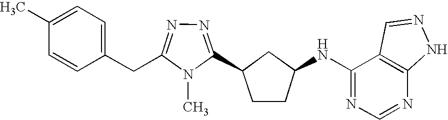

### Step A: tert-butyl {(1S,3R)-3-[(methylamino)carbonyl]cyclopentyl}carbamate

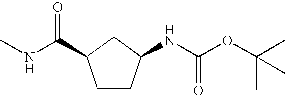

To a solution of (1R,3S)-3-[(tert-butoxycarbonyl)amino]cyclopentanecarboxylic acid (1.50 g, 6.54 mmol) and HOBt (1.77 g, 13.1 mmol) in DMF (20 mL) at room temperature was added EDC (2.51 g, 13.1 mmol). After 60 minutes, methylamine (610 mg, 19.6 mmol) was added to the reaction. After 17 hours, the reaction was poured into ethyl acetate. The organic layer was washed with water, brine, dried over sodium sulfate, filtered and concentrated to give the title compound (1.45 g) as a white solid. This material was sufficiently pure for the next step. ¹H NMR (400 MHz, CDCl₃): 5.70 (s, 1H), 5.51 (s, 1H), 4.09 (s, 1H), 2.82 (d, J=4.8 Hz, 3H), 2.63-2.57 (m, 1H), 2.11-2.04 (m, 1H), 1.94-1.88 (m, 2H), 1.84-1.72 (m, 3H), 1.44 (s, 9H).

### Step B: tert-butyl [(1S,3R)-3-(methylaminocarbonothioyl)cyclopentyl]carbamate

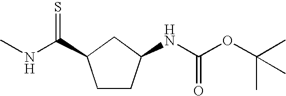

To a suspension of tert-butyl {(1S,3R)-3-[(methylamino)carbonyl]cyclopentyl}carbamate (1.45 g, 5.99 mmol) in THF (30 mL) at room temperature was added Lawesson's reagent (1.58 g, 3.90 mmol). After 24 hours, the reaction was concentrated and dissolved in ethyl acetate. The organic layer was washed with saturated sodium bicarbonate, brine, dried over sodium sulfate, filtered and concentrated. Purification by silica gel chromatography (15% ethyl acetate/hexanes→90% ethyl acetate/hexanes) gave the title compound (1.03 g) as a white solid. ¹H NMR (400 MHz, CDCl₃): 7.48 (s, 1H), 5.34 (s, 1H), 4.05 (m, 1H), 3.19 (d, J=4.8 Hz, 3H), 3.00-2.94 (m, 1H), 2.30-2.23 (m, 1H), 2.13-1.94 (m, 3H), 1.88-1.79 (m, 2H), 1.44 (s, 9H).

### Step C: tert-butyl [(1S,3R)-3-(5-benzyl-4-methyl-4H-1,2,4-triazol-3-yl)cyclopentyl]carbamate

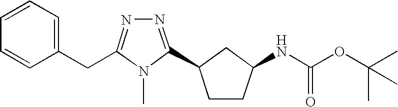

To a solution of tert-butyl [(1S,3R)-3-(methylaminocarbonothioyl)cyclopentyl]carbamate (200 mg, 0.774 mmol) in THF (4.0 mL) was added 2-phenylacetohydrazide (128 mg, 0.851 mmol) and mercuric(II) acetate (271 mg, 0.851 mmol) at room temperature. After 72 hours, the reaction was concentrated and suspended in ether. The mixture was filtered, and the filtrate was concentrated to give the title compound (270 mg) as a colorless oil. MS 357 (M+1).

### Step D: [(1S,3R)-3-(5-benzyl-4-methyl-4H-1,2,4-triazol-3-yl)cyclopentyl]amine

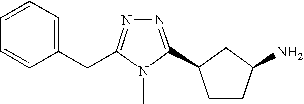

To tert-butyl [(1S,3R)-3-(5-benzyl-4-methyl-4H-1,2,4-triazol-3-yl)cyclopentyl]carbamate (276 mg, 0.774 mmol) was added trifluoroacetic acid (1.5 mL) and the resulting solution was stirred at room temperature. After 10 minutes, the reaction was concentrated and the residue was loaded onto an SCX ion exchange resin as a solution in acetonitrile/water (1:1) and eluted with ammonia in ethanol solution to give the title compound (118 mg) as a colorless oil. MS 257 (M+1).

### Step E: N-[(1S,3R)-3-(5-benzyl-4-methyl-4H-1,2,4-triazol-3-yl)cyclopentyl]-1H-pyrazolo[3,4-d]pyrimidin-4-amine

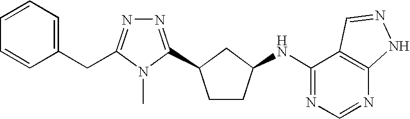

To solution of [(1S,3R)-3-(5-benzyl-4-methyl-4H-1,2,4-triazol-3-yl)cyclopentyl]amine (118 mg, 0.460 mmol) in 1-butanol (1.0 mL) was added DIPEA (1.0 mL) and 4-chloro-1-(tetrahydro-pyran-2-yl)-1H-pyrazolo[3,4-d]pyrimidine (110 mg, 0.460 mmol) and the solution was heated at 120° C. for 1 hour. The mixture was cooled and concentrated under reduced pressure. The resulting residue was dissolved in methanol (3.0 mL) and 6N HCl (1.0 mL) and was stirred at room temperature for 1 hour, at which time the reaction was quenched with saturated sodium bicarbonate. The solution was extracted with tetrahydrofuran, and the organic layer was washed with brine, dried over sodium sulfate, filtered and concentrated to dryness under reduced pressure. Purification by reverse phase chromatography (Waters XTerra MSC8, 5% acetonitrile/0.1% trifluoroacetice acid/water→95% acetonitrile/0.1% trifluoroacetice acid/water) gave the title compound (76 mg) as a white solid. HRMS (M+H⁺): calculated=375.2040, observed=375.2049; ¹H NMR (400 MHz, CD₃OD): 8.64 (s, 1H), 8.55 (s, 1H), 7.44-7.33 (m, 5H), 4.9 (m, 1H), 4.43 (s, 2H), 3.77 (s, 3H), 3.68-3.64 (m, 1H), 2.83-2.80 (m, 1H), 2.40-2.34 (m, 2H), 2.24-2.16 (m, 2H), 2.05-2.01 (m, 1H).

### EXAMPLE 50

### N-[(1S,3S)-3-(5-benzyl-4-methyl-4H-1,2,4-triazol-3-yl)cyclopentyl]-1H-pyrazolo[3,4-d]pyrimidin-4-amine

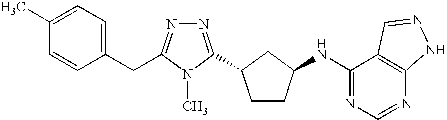

### Step A: tert-butyl {(1S,3S)-3-[(methylamino)carbonyl]cyclopentyl}carbamate

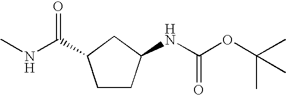

To a solution of (1S,3S)-3-[(tert-butoxycarbonyl)amino]cyclopentanecarboxylic acid (0.910 g, 3.95 mmol) and HOBt (1.21 g, 7.90 mmol) in anhydrous DMF (20 mL) at room temperature under N₂ was added EDC (1.52 g, 7.90 mmol). After 60 minutes, 2.0 M methylamine in THF (0.430 mL, 11.9 mmol) was added to the reaction. After 14 hours, the reaction was poured into ethyl acetate. The organic layer was washed with water, brine, dried over sodium sulfate, filtered and concentrated to give the title compound (0.95 g) as a white solid. This material was sufficiently pure for the next step. MS 485.4 (2M+1) and 187.3 (M−55).

### Step B: tert-butyl [(1S,3S)-3-(methylaminocarbonothioyl)cyclopentyl]carbamate

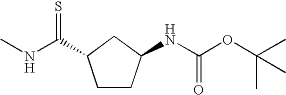

To a suspension of tert-butyl {(1S,3S)-3-[(methylamino)carbonyl]cyclopentyl}carbamate (0.950 g, 3.91 mmol) in anhydrous THF (30 mL) at room temperature was added Lawesson's reagent (1.09 g, 2.69 mmol), while stirring under N₂. After 6 days, the reaction was concentrated and dissolved in ethyl acetate. The organic layer was washed with saturated sodium bicarbonate, brine, dried over sodium sulfate, filtered and concentrated. Purification by silica gel chromatography (25% ethyl acetate/hexanes→90% ethyl acetate/hexanes) gave the title compound (0.66 g) as a white solid. HRMS (M+H⁺): calculated=259.1478, observed=259.1480.

### Step C: tert-butyl [(1S,3S)-3-(5-benzyl-4-methyl-4H-1,2,4-triazol-3-yl)cyclopentyl]carbamate

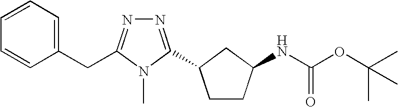

To a solution of tert-butyl [(1S,3S)-3-(methylaminocarbonothioyl)cyclopentyl]carbamate (0.20 g, 0.78 mmol) in anhydrous THF (4.0 mL) was added 2-phenylacetohydrazide (0.13 g, 0.86 mmol) and mercuric(II) acetate (0.27 g, 0.86 mmol) at room temperature under N₂. After 14 hours, the reaction mixture was filtered and concentrated under reduced pressure. The residue was suspended in diethyl ether, filtered, and concentrated under reduced pressure to yield the title compound (0.32 g) as a colorless oil. MS 357.4 (M+1).

### Step D: [(1S,3S)-3-(5-benzyl-4-methyl-4H-1,2,4-triazol-3-yl)cyclopentyl]amine

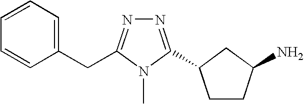

To tert-butyl [(1S,3S)-3-(5-benzyl-4-methyl-4H-1,2,4-triazol-3-yl)cyclopentyl]carbamate (0.28 g, 0.78 mmol) was added trifluoroacetic acid (5.0 mL) and the resulting solution was stirred at room temperature. After 10 minutes, the reaction was concentrated and the residue was loaded onto an SCX ion exchange resin as a solution in acetonitrile/water (1:1) and eluted with ammonia in ethanol solution. The solution was concentrated to an oil, dissolved in ethyl acetate, treated with anhydrous hydrochloric acid in ethyl acetate, and concentrated to yield the hydrochloride salt of the title compound (0.20 g) as a tan solid. HRMS (M+H⁺): calculated=257.1761, observed=257.1765.

### Step E: N-[(1S,3S)-3-(5-benzyl-4-methyl-4H-1,2,4-triazol-3-yl)cyclopentyl]-1H-pyrazolo[3,4-d]pyrimidin-4-amine

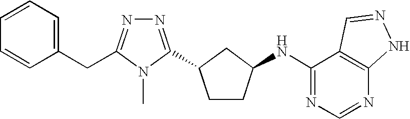

To a solution of [(1S,3S)-3-(5-benzyl-4-methyl-4H-1,2,4-triazol-3-yl)cyclopentyl]amine (0.14 g, 0.49 mmol) in 1-butanol (1.5 mL) was added DIPEA (1.5 mL) and 4-chloro-1-(tetrahydro-pyran-2-yl)-1H-pyrazolo[3,4-d]pyrimidine (0.13 g, 0.54 mmol) and the solution was heated at 150° C. for 15 minutes under microwave irradiation. The mixture was cooled and concentrated under reduced pressure. The resulting residue was dissolved in methanol (3.0 mL) and 6N HCl (1.5 mL) and was stirred at room temperature for 1 hour, at which time the reaction was quenched with saturated sodium bicarbonate. The solution was extracted with ethyl acetate, and the organic layer was washed with brine, dried over sodium sulfate, filtered and concentrated to dryness under reduced pressure. Purification by reverse phase chromatography (Waters XTerra MSC8, 5% acetonitrile/0.1% trifluoroacetic acid/water→95% acetonitrile/0.1% trifluoroacetice acid/water) gave the title compound which was dissolved in ethyl acetate, treated with anhydrous hydrochloric acid in ethyl acetate, and concentrated to yield the hydrochloride salt of the title compound (0.04 g) as a solid. HRMS (M+H⁺): calculated=375.2040, observed=375.2026; ¹H NMR (400 MHz, CD₃OD): 8.71 (s, 1H), 8.53 (s, 1H), 7.46-7.27 (m, 5H), 5.02-4.90 (m, 1H), 4.43 (s, 2H), 3.78 (s, 3H), 2.61-2.27 (m, 4H), 2.13-1.90 (m, 2H).

### EXAMPLE 51

### N-[(3S)-1-(3-phenyl-1,2,4-oxadiazol-5-yl)pyrrolidin-3-yl]-1H-pyrazolo[3,4-d]pyrimidin-4-amine

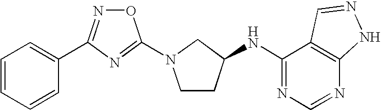

### Step A: 3-phenyl-5-(trichloromethyl)-1,2,4-oxadiazole

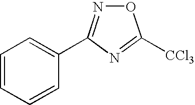

To a solution of N′-hydroxybenzenecarboximidamide (5.00 g, 36.7 mmol) in toluene (100 mL) was added trichloroacetic anhydride (11.3 g, 36.7 mmol) and the solution was heated to 120° C. After 2.5 hours, the reaction was cooled to room temperature and poured into water. The aqueous layer was extracted with ethyl acetate and the combined organics were washed with saturated bicarbonate, dried over sodium sulfate, filtered and concentrated. Purification by silica gel chromatography (0.5% ethyl acetate/hexanes→25% ethyl acetate/hexanes) gave the title compound (7.80 g) as a colorless oil. ¹H NMR (400 MHz, DMSO-d₆): 8.06 (d, J=7.2 Hz, 2H), 7.70-7.61 (m, 3H).

### Step B: tert-butyl [(3S)-1-(3-phenyl-1,2,4-oxadiazol-5-yl)pyrrolidin-3-yl]carbamate

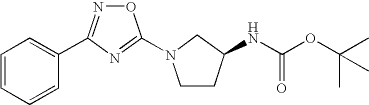

To a solution of tert-butyl (3S)-pyrrolidin-3-ylcarbamate (1.00 g, 5.37 mmol) in methanol (10 mL) was added 3-phenyl-5-(trichloromethyl)-1,2,4-oxadiazole (1.41 g, 5.37 mmol) at room temperature. After 10 days, the reaction was concentrated and dissolved in ethyl acetate. The organic layer was washed with water, 1M citric acid, saturated sodium bicarbonate, brine, dried over sodium sulfate, filtered and concentrated. Purification by silica gel chromatography (15% ethyl acetate/5% methylene chloride/hexanes→95% ethyl acetate/5% methylene chloride/hexanes) gave the title compound (233 mg) as a white solid. HRMS (M+H): calculated=331.1765, observed=331.1755; ¹H NMR (400 MHz, DMSO-d₆): 7.91 (d, J=8.0 Hz, 2H), 7.54-7.49 (m, 3H), 7.31 (m, 1H), 4.14 (m, 1H), 3.75-3.58 (m, 3H), 3.41-3.38 (m, 1H), 2.18-2.14 (m, 1H), 1.94-1.89 (m, 1H), 1.40 (s, 9H).

### Step C: (3S)-1-(3-phenyl-1,2,4-oxadiazol-5-yl)pyrrolidin-3-amine

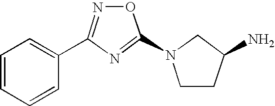

To tert-butyl [(3S)-1-(3-phenyl-1,2,4-oxadiazol-5-yl)pyrrolidin-3-yl]carbamate (215 mg, 0.651 mmol) was added trifluoroacetic acid (1.5 mL) and the resulting solution was stirred at room temperature. After 1 hour, the reaction was concentrated and the residue was dissolved in ethyl acetate, treated with anhydrous hydrochloric acid and concentrated. The solid was suspended in ether/hexane and filtered to give the title compound (173 mg) as a white solid. HRMS (M+H⁺): calculated=231.1241, observed=231.1242; ¹H NMR (400 MHz, DMSO-d₆): 8.31 (br s, 3H), 7.92 (dd, J=1.6, 8.0 Hz, 2H), 7.55-7.49 (m, 3H), 3.97 (m, 1H), 3.87-3.65 (m, 4H), 2.36-2.33 (m, 1H), 2.19-2.17 (m, 1H).

### Step D: N-[(3S)-1-(3-phenyl-1,2,4-oxadiazol-5-yl)pyrrolidin-3-yl]-1H-pyrazolo[3,4-d]pyrimidin-4-amine

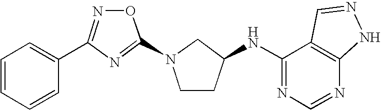

To a solution of (3S)-1-(3-phenyl-1,2,4-oxadiazol-5-yl)pyrrolidin-3-amine (60 mg, 0.22 mmol) in 1-butanol (1.1 mL) was added DIPEA (1.1 mL) and 4-chloro-1-(tetrahydro-pyran-2-yl)-1H-pyrazolo[3,4-d]pyrimidine (54 mg, 0.22 mmol) and the solution was heated at 150° C. for 15 minutes under microwave irradiation. The mixture was cooled and concentrated under reduced pressure. The resulting residue was dissolved in methanol (3 mL) and 6N HCl (0.5 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (3% isopropanol/methylene chloride→35% isopropanol/methylene chloride) gave the title compound (21 mg) as a white solid. HRMS (M+H⁺): calculated=349.1520, observed=349.1560; ¹H NMR (400 MHz, DMSO-d₆): 13.46 (s, 1H), 8.38 (d, J=5.2 Hz, 1H), 8.29 (s, 1H), 8.13 (s, 1H), 7.92 (d, J=8.0 Hz, 2H), 7.54-7.51 (m, 3H), 4.86 (m, 1H), 4.00-3.96 (m, 1H), 3.79-3.73 (m, 2H), 3.64-3.61 (m, 1H), 2.39-2.36 (m, 1H), 2.18-2.16 (m, 1H).

### EXAMPLE 52

### N-{(3S)-1-[3-(4-methylphenyl)-1,2,4-oxadiazol-5-yl]pyrrolidin-3-yl}-1H-pyrazolo[3,4-d]pyrimidin-4-amine

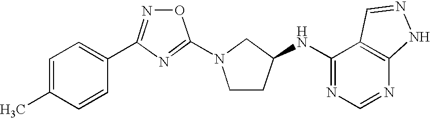

### Step A: 3-(4-methylphenyl)-5-(trichloromethyl)-1,2,4-oxadiazole

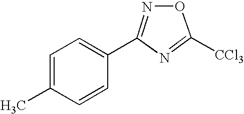

To a solution of N′-hydroxy-4-methylbenzenecarboximidamide (4.29 g, 28.5 mmol) in toluene (100 mL) was added trichloroacetic anhydride (8.81 g, 28.5 mmol) and the solution was heated to 120° C. After 2.5 hours, the reaction was cooled to room temperature and poured into water. The aqueous layer was extracted with ethyl acetate and the combined organics were washed with saturated bicarbonate, dried over sodium sulfate, filtered and concentrated. Purification by silica gel chromatography (0.5% ethyl acetate/hexanes→25% ethyl acetate/hexanes) gave the title compound as a colorless oil. MS 279.1 (M+2).

### Step B: tert-butyl {(3S)-1-[3-(4-methylphenyl)-1,2,4-oxadiazol-5-yl]pyrrolidin-3-yl}carbamate

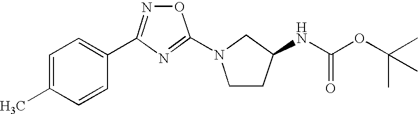

To a solution of 3-(4-methylphenyl)-5-(trichloromethyl)-1,2,4-oxadiazole (0.390 g, 1.42 mmol) in N-methylpyrrolidinone (2 mL) was added tert-butyl (3S)-pyrrolidin-3-ylcarbamate (0.590 g, 3.17 mmol) and the resulting solution was heated to 150° C. under microwave irradiation. The reaction was cooled to room temperature and extracted with ether. The organic layer was washed with 1M citric acid, water, brine, dried over sodium sulfate, filtered and concentrated. Purification by silica gel chromatography (1% ethyl acetate/hexanes→50% ethyl acetate/hexanes) gave the title compound as a white solid. HRMS (M+H⁺): calculated=345.1921, observed=345.1931.

### Step C: (3S)-1-[3-(4-methylphenyl)-1,2,4-oxadiazol-5-yl]pyrrolidin-3-amine

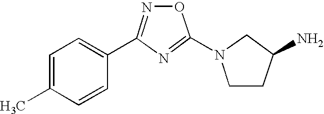

To tert-butyl {(3S)-1-[3-(4-methylphenyl)-1,2,4-oxadiazol-5-yl]pyrrolidin-3-yl}carbamate (296 mg, 0.859 mmol) was added trifluoroacetic acid (0.5 mL) and the resulting solution was stirred at room temperature. After 20 minutes, the reaction was concentrated and the residue was dissolved in ethyl acetate. The solution was treated with anhydrous hydrochloric acid and filtered to give the hydrochloride salt of the title compound (239 mg) as a white solid. HRMS (M+H⁺): calculated=245.1397, observed=245.1381.

### Step D: N-{(3S)-1-[3-(4-methylphenyl)-1,2,4-oxadiazol-5-yl]pyrrolidin-3-yl}-1H-pyrazolo[3,4-d]pyrimidin-4-amine

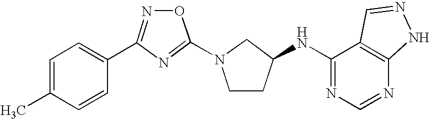

To solution of (3S)-1-[3-(4-methylphenyl)-1,2,4-oxadiazol-5-yl]pyrrolidin-3-amine (139 mg, 0.500 mmol) in 1-butanol (2.0 mL) was added DIPEA (2.0 mL) and 4-chloro-1-(tetrahydro-pyran-2-yl)-1H-pyrazolo[3,4-d]pyrimidine (126 mg, 0.530 mmol) and the solution was heated at 100° C. for 4 hours. The mixture was cooled and concentrated under reduced pressure. The resulting residue was dissolved in methanol (2 mL) and 6N HCl (1 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound as a white solid. HRMS (M+H⁺): calculated=363.1677, observed=363.1680.

The Examples in Table 3 were prepared essentially following the procedures outlined for the preparation of Examples 51 and 52.

TABLE 3

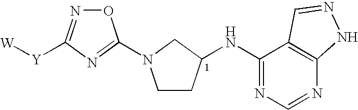

| | | | | HRMS (M + H⁺): |
|---|---|---|---|---|
| | | | | Observed |
| Examples | C-1 | Y | W | [Calculated] |
| 53 | R | — | 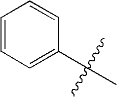 | 349.1524 [349.1520] |
| 54 | R | — | 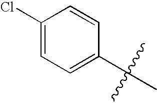 | 383.1124 [383.1130] |
| 55 | S | — | 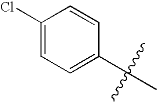 | 383.1118 [383.1130] |
| 56 | S | CH₂ | 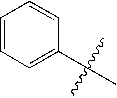 | 363.1675 [363.1677] |

### EXAMPLE 57

### N-{(3S)-1-[5-(4-methylbenzyl)-1,3,4-thiadiazol-2-yl]pyrrolidin-3-yl}-1H-pyrazolo[3,4-d]pyrimidin-4-amine

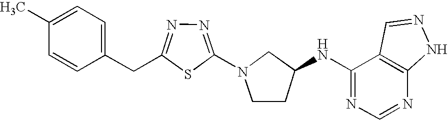

### Step A: 5-(4-methylbenzyl)-1,3,4-thiadiazol-2-amine

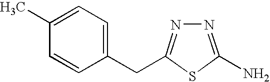

To a solution of (4-methylphenyl)acetonitrile (19.0 g, 144 mmol) in trifluoroacetic acid (225 mL) was added hydrazinecarbothioamide (14.9 g, 163 mmol), and the mixture was stirred at 110° C. for 3 hours. The reaction was cooled to room temperature, poured into water, and allowed to stir until solid formed. The suspension was filtered to give the title compound (10.0 g). This material was sufficiently pure for the next step. MS 206 (M+H⁺).

### Step B: 2-chloro-5-(4-methylbenzyl)-1,3,4-thiadiazole

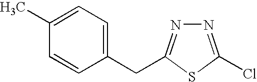

To a stirred solution of 5-(4-methylbenzyl)-1,3,4-thiadiazol-2-amine (5.02 g, 24.5 mmol) in concentrated hydrochloric acid at 0° C. was added CuCl (242 mg, 2.45 mmol) and a solution of sodium nitrate (42.2 g, 611 mmol) in water (250 mL) dropwise. Following completion of the addition, the reaction was heated to 90° C. for 1 hour. The reaction was cooled to room temperature and extracted with ethyl acetate. The combined organic layers were dried over sodium sulfate, filtered, and concentrated under reduced pressure to give the title compound as a red oil (2.10 g). This material was sufficiently pure for the next step. MS 205 (M+1).

### Step C: tert-butyl {(3S)-1-[5-(4-methylbenzyl)-1,3,4-thiadiazol-2-yl]pyrrolidin-3-yl}carbamate

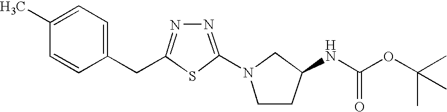

To a solution of 2-chloro-5-(4-methylbenzyl)-1,3,4-thiadiazole (1.06 g, 4.73 mmol) in DMF (10 mL) was added tert-butyl (3S)-pyrrolidin-3-ylcarbamate (0.880 g, 4.73 mmol) and DIPEA (10 mL) and the reaction was heated to 100° C. for 1 hour. The reaction was concentrated under reduced pressure and dissolved in ethyl acetate. The organic layer was washed with saturated sodium bicarbonate, dried over sodium sulfate, filtered, and concentrated under reduced pressure. Purification by silica gel chromatography (1% ethyl acetate/hexane→50% ethyl acetate/hexane) gave the title compound (1.05 g) as a white solid. MS=375 (M+1).

### Step D: (3S)-1-[5-(4-methylbenzyl)-1,3,4-thiadiazol-2-yl]pyrrolidin-3-amine

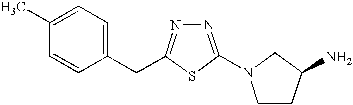

To tert-butyl {(3S)-1-[5-(4-methylbenzyl)-1,3,4-thiadiazol-2-yl]pyrrolidin-3-yl}carbamate (1.05 g, 2.80 mmol) was added trifluoroacetic acid (5.0 mL) and the resulting solution was stirred at room temperature. After 20 minutes, the reaction was concentrated and the residue was dissolved in ethyl acetate. The solution was treated with anhydrous hydrochloric acid and filtered to give the title compound (500 mg). MS=275 (M+1).

### Step E: N-{(3S)-1-[5-(4-methylbenzyl)-1,3,4-thiadiazol-2-yl]pyrrolidin-3-yl}-1H-pyrazolo[3,4-d]pyrimidin-4-amine

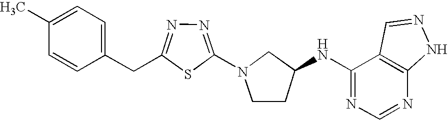

To solution of (3S)-1-[5-(4-methylbenzyl)-1,3,4-thiadiazol-2-yl]pyrrolidin-3-amine (81 mg, 0.24 mmol) in 1-butanol (0.5 mL) was added DIPEA (0.5 mL) and 4-chloro-1-(tetrahydro-pyran-2-yl)-1H-pyrazolo[3,4-d]pyrimidine (58 mg, 0.24 mmol) and the solution was heated at 150° C. for 10 minutes under microwave irradiation. The mixture was cooled and concentrated under reduced pressure. The resulting residue was dissolved in methanol (2 mL) and 6N HCl (1 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (50 mg) as a white solid. HRMS (M+H⁺): calculated=393.1592, observed=393.1605.

### EXAMPLE 58

### N-[(3S)-1-(5-phenyl-1,2,4-oxadiazol-3-yl)pyrrolidin-3-yl]-1H-pyrazolo[3,4-d]pyrimidin-4-amine

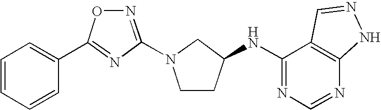

### Step A: tert-butyl [(3S)-1-cyanopyrrolidin-3-yl]carbamate

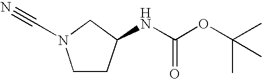

To a solution of tert-butyl (3S)-pyrrolidin-3-ylcarbamate (8.02 g, 43.0 mmol) in methanol (100 ml) and sodium acetate (7.05 g, 86 mmol) was added cyanogen bromide (4.56 g, 43.0 mmol) at 0° C. The reaction was allowed to slowly warm to room temperature over 5 hours. The reaction was concentrated, and the resulting residue was dissolved in ethyl acetate. The organic layer was washed with saturated sodium bicarbonate, dried over sodium sulfate, filtered and concentrated to a thick oil which solidified upon standing to give the title compound (6.0 g). This material was sufficiently pure for the next step. MS 212 (M+1).

### Step B: tert-butyl {(3S)-1-[(E)-amino(hydroxyimino)methyl]pyrrolidin-3-yl}carbamate

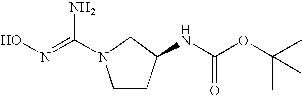

To a solution of tert-butyl [(3S)-1-cyanopyrrolidin-3-yl]carbamate (262 mg, 1.07 mmol) in ethanol (5 mL) and water (1 mL) was added hydroxylamine hydrochloride (181 mg, 1.07 mmol) and sodium carbonate (1.13 g, 10.7 mmol), and the resulting solution was heated to 85° C. After 3 hours, the reaction was cooled to room temperature, concentrated and dissolved in ethyl acetate. The organic layer was washed with 1M sodium hydroxide, water, brine, dried over sodium sulfate, filtered and concentrated to give title compound (0.500 g). This material was sufficiently pure for the next step. MS 245 (M+1).

### Step C: tert-butyl [(3S)-1-(5-phenyl-1,2,4-oxadiazol-3-yl)pyrrolidin-3-yl]carbamate

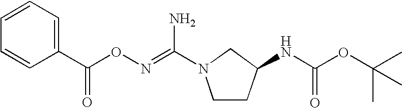

To a solution of benzoic acid (128 mg, 1.05 mmol) in methylene chloride (10 mL) was added HOBT (142 mg, 1.05 mmol) and EDC (201 mg, 1.05 mmol) and the reaction was stirred for 1 hour, at which time tert-butyl {(3S)-1-[(E)-amino(hydroxyimino)methyl]pyrrolidin-3-yl}carbamate (256 mg, 1.05 mmol) was added. After 14 hours, the reaction was poured into saturated sodium bicarbonate and extracted with methylene chloride. The organic layer dried over sodium sulfate, filtered and concentrated to give the title compound (280 mg). This material was sufficiently pure for the next step. MS 349 (M+1).

### Step D: tert-butyl [(3S)-1-(5-phenyl-1,2,4-oxadiazol-3-yl)pyrrolidin-3-yl]carbamate

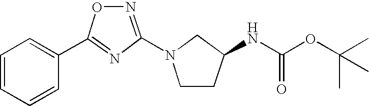

To a solution of tert-butyl [(3S)-1-(5-phenyl-1,2,4-oxadiazol-3-yl)pyrrolidin-3-yl]carbamate (280 mg, 0.804 mmol) in ethanol (5 mL) and water (1 mL) was added sodium acetate (659 mg, 8.04 mmol) and the resulting solution was heated to 85° C. After 3 hours, the reaction was cooled to room temperature, concentrated and dissolved in ethyl acetate. The organic layer was washed with 1M sodium hydroxide, water, brine, dried over sodium sulfate, filtered and concentrated. Purification by silica gel chromatography (1% ethyl acetate/hexanes→50% ethyl acetate/hexanes) gave the title compound (200 mg) as a white solid. MS 331 (M+1).

### Step E: (3S)-1-(5-phenyl-1,2,4-oxadiazol-3-yl)pyrrolidin-3-amine

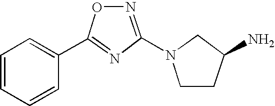

To tert-butyl [(3S)-1-(5-phenyl-1,2,4-oxadiazol-3-yl)pyrrolidin-3-yl]carbamate (200 mg, 0.605 mmol) was added trifluoroacetic acid (0.5 mL) and the resulting solution was stirred at room temperature. After 20 minutes, the reaction was concentrated and the residue was dissolved in ethyl acetate. The solution was treated with anhydrous hydrochloric acid and filtered to give the title compound (100 mg). MS 267 (M+1).

### Step F: N-[(3S)-1-(5-phenyl-1,2,4-oxadiazol-3-yl)pyrrolidin-3-yl]-1H-pyrazolo[3,4-d]pyrimidin-4-amine

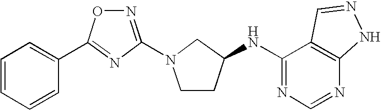

To a solution of (3S)-1-(5-phenyl-1,2,4-oxadiazol-3-yl)pyrrolidin-3-amine (300 mg, 1.12 mmol) in 1-butanol (0.5 mL) was added DIPEA (0.5 mL) and 4-chloro-1-(tetrahydro-pyran-2-yl)-1H-pyrazolo[3,4-d]pyrimidine (268 mg, 1.12 mmol) and the solution was heated at 150° C. for 10 minutes under microwave irradiation. The mixture was cooled and concentrated under reduced pressure. The resulting residue was dissolved in methanol (10 mL) and 6N HCl (3 mL) and was heated at 60° C. for 1 hour, at which time the reaction was cooled and quenched with saturated sodium bicarbonate. The solution was extracted with ethyl acetate, and the organic layer was dried over sodium sulfate, filtered and concentrated to dryness under reduced pressure. Purification by silica gel chromatography (1% isopropanol/methylene chloride→25% isopropanol/methylene chloride) gave the title compound (150 mg) as a white solid. HRMS (M+H⁺): calculated=349.1520, observed=349.1518; ¹H NMR (400 MHz, DMSO-d₆): 8.70-8.55 (m, 2H), 8.06-8.02 (m, 2H), 7.72-7.65 (m, 1H), 7.64-7.58 (m, 2H), 4.90 (m, 1H), 3.90-3.85 (m, 1H), 3.75-3.65 (m, 1H), 3.65-3.55 (m, 1H), 2.45-2.40 (m, 1H), 2.30-2.20 (m, 1H).

The Examples in Table 4 were prepared essentially following the procedure outlined for the preparation of Example 58.

TABLE 4

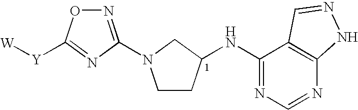

| | | | | HRMS (M + H⁺): |
|---|---|---|---|---|
| | | | | Observed |
| Examples | C-1 | Y | W | [Calculated] |
| 59 | R | — | 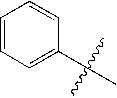 | 349.1518 [349.1520] |
| 60 | S | — | 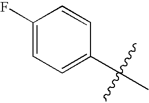 | 367.1416 [367.1426] |
| 61 | S | — | 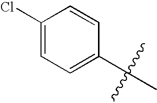 | 383.1116 [383.1130] |
| 62 | S | — | 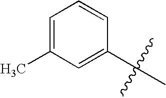 | 363.1683 [363.1677] |
| 63 | S | — | 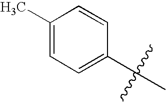 | 363.1676 [363.1677] |
| 64 | S | — | 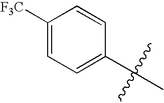 | 417.1383 [417.1394] |
| 65 | S | — | 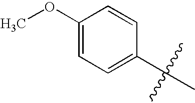 | 379.1625 [379.1629] |
| 66 | R | CH₂ | 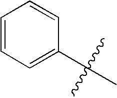 | 363.1686 [363.1677] |
| 67 | S | CH₂ | 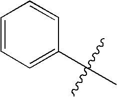 | 363.1670 [363.1677] |
| 68 | S | CH₂ | 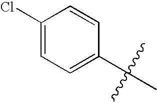 | 397.1280 [397.1287] |
| 69 | S | CH₂ | 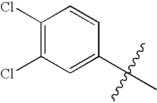 | 431.0895 [431.0897] |
| 70 | S | CH₂ | 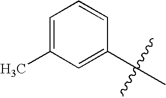 | 377.1819 [377.1833] |
| 71 | S | CH₂ | 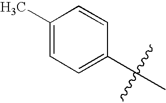 | 377.1816 [377.1833] |
| 72 | S | CH₂ | 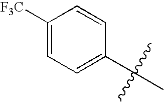 | 431.1551 |
| 73 | S | CH₂ | 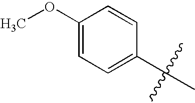 | 393.1769 [393.1782] |
| 74 | S | 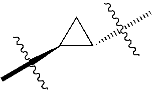 | 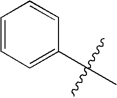 | 389.1835 [389.1833] |
| 75 | S | CH₂CH₂ | 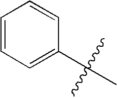 | 377.1832 [377.1833] |
| 76 | S | — | 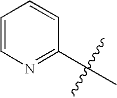 | 350.1485 [350.1473] |
| 77 | S | — | 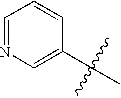 | 350.1481 [350.1473] |
| 78 | S | — | 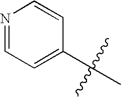 | 350.1471 [350.1473] |
| 79 | S | — | 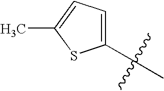 | 369.1240 [369.1241] |
| 80 | S | — | 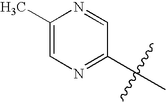 | 365.1564 [365.1582] |

### EXAMPLE 81

### N-{(3R)-1-[1-methyl-5-(4-methylphenyl)-1H-1,2,4-triazol-3-yl]pyrrolidin-3-yl}-1H-pyrazolo[3,4-d]pyrimidin-4-amine

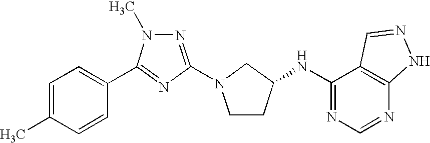

### Step A: 1,1-di(1H-1,2,3-benzotriazol-1-yl)methanimine

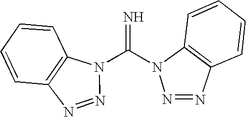

To a stirred solution of benzotriazole (11.2 g, 94.4 mmol) in ethanol (150 mL) at 0° C. was added a solution of cyanogen bromide (5.00 g, 47.2 mmol) in acetone (20 mL) dropwise, followed by aqueous sodium hydroxide (0.05 mmol of NaOH in 20 ml of H₂O). The reaction was stirred for 5 hours and the resulting white precipitate was collected by filtration. The solid was washed with cold ethanol to give title compound (10.0 g). This material was sufficiently pure for the next step. MS 264 (M+1).

### Step B: tert-butyl {(3R)-1-[1H-1,2,3-benzotriazol-1-yl(imino)methyl]pyrrolidin-3-yl}carbamate

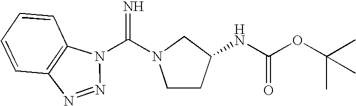

To a stirred solution of 1,1-di(1H-1,2,3-benzotriazol-1-yl)methanimine (1.0 g, 3.8 mmol) in dry THF (75 mL) was added (3S)-(−)-3-(tert-butoxycarbonylamino)pyrrolidine (0.71 g, 3.8 mmol). After 12 hours, the reaction was concentrated and the residue was dissolved in methylene chloride. The organic layer was washed with saturated sodium carbonate, dried over sodium sulfate, filtered and concentrated to give the title compound (850 mg) as a white solid. This material was sufficiently pure for the next step. MS 331 (M+1).

### Step C: (3R)-1-[1-methyl-5-(4-methylphenyl)-1H-1,2,4-triazol-3-yl]pyrrolidin-3-amine

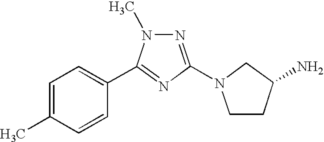

To a stirred solution of tert-butyl {(3R)-1-1H-1,2,3-benzotriazol-1-yl(imino)methyl]pyrrolidin-3-yl}carbamate (0.500 g, 1.51 mmol) in methylene chloride (25 mL) was added DIPEA (0.293 g, 2.27 mmol) and p-toluoyl chloride (0.234 g, 1.51 mmol) at room temperature. After 1 hour, methylhydrazine (0.087 g, 1.89 mmole) was added. After 1 hour, the reaction was concentrated under reduced pressure and the resulting residue was dissolved in trifluoroacetic acid (5 mL) at room temperature. The solution was concentrated, dissolved in ethyl acetate, and treated with anhydrous hydrochloric acid. The solution was concentrated and purified by recrystallization (ethyl acetate/methanol) to give the title compound (150 mg) as a white solid. HRMS (M+H⁺): calculated=258.1713, observed=258.1719.

### Step D: N-{(3R)-1-[1-methyl-5-(4-methylphenyl)-1H-1,2,4-triazol-3-yl]pyrrolidin-3-yl}-1H-pyrazolo[3,4-d]pyrimidin-4-amine

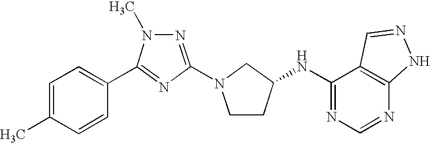

To a stirred solution of (3R)-1-[1-methyl-5-(4-methylphenyl)-1H-1,2,4-triazol-3-yl]pyrrolidin-3-amine (150 mg, 0.551 mmol) in DIPEA (3 mL) and 1-butanol (3 mL) was added 4-chloro-1-tetrahydro-2H-pyran-2-yl-1H-pyrazolo[3,4-d]pyrimidine (122 mg, 0.551 mmol) and the reaction was stirred at 80° C. for 2 hours. The reaction was concentrated under vacuum, dissolved in ethyl acetate, and treated with anhydrous hydrochloric acid in ethyl acetate for 1 hour at room temperature. The reaction was poured into saturated sodium bicarbonate and extracted with ethyl acetate. The organic layer was dried over sodium sulfate, filtered and concentrated. Purification by reverse phase chromatography (Waters XTerra, MS C8, 5-95% acetonitrile/0.1% trifluoroacetic acid/water) gave the title compound (100 mg) as a white solid. HRMS (M+H⁺): calculated=376.1993, observed=376.2001; ¹H NMR (400 MHz, CD₃OD): 8.82-8.52 (m, 2H), 7.64-7.6 (m, 2H), 7.38-7.36 (m, 2H), 4.9-4.85 (m, 1H), 3.70-3.60 (m, 1H), 3.58-3.50 (m, 2H), 2.4 (s, 3H), 2.25-2.18 (m, 1H).

1. A compound of Formula I: 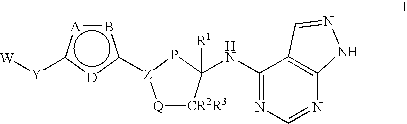
wherein:
W is aryl or heteroaryl, unsubstituted or substituted with 1-5 substituents independently selected from halogen, C₁₋₆ alkyl optionally substituted with one or more substituents selected from halogen, hydroxyl and C₁₋₄ alkoxy, C₁₋₄ alkoxy, C₃₋₆ cycloalkyl, cyano and N(R⁴)₂;
Y is absent or is selected from C₁₋₃ alkyl optionally substituted with one or more substituents selected from halogen, hydroxyl and C₁₋₄ alkoxy, cyclopropyl and C(O);
Z is N and P and Q are C(R⁴)₂, or Z is CR⁵ and P and Q are C(R⁵)₂;
A, B and D are each independently selected from O, CR⁶, S and NR⁶;
R¹, R⁴ and R⁶ are each independently selected from hydrogen and C₁₋₄ alkyl optionally substituted with one or more of halogen and hydroxyl; and
R², R³, and R⁵ are each independently selected from hydrogen, halogen, hydroxyl, C₁₋₄ alkyl optionally substituted with one or more substituents selected from halogen and hydroxyl, C₁₋₄ alkoxy, cyano and N(R⁴)₂;
and pharmaceutically acceptable salts and individual enantiomers and stereoisomers thereof.

2. A compound of claim 1 having Formula Ia: 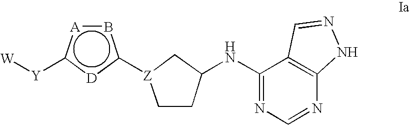
wherein:
W is aryl or heteroaryl, unsubstituted or substituted with 1-5 substituents independently selected from halogen, C₁₋₆ alkyl optionally substituted with one or more substituents selected from halogen, hydroxyl and C₁₋₄ alkoxy, C₁₋₄ alkoxy, C₃₋₆ cycloalkyl, cyano and N(R⁴)₂;
Y is absent or is selected from C₁₋₃ alkyl optionally substituted with one or more substituents selected from halogen, hydroxyl and C₁₋₄ alkoxy, cyclopropyl and C(O);
Z is N or Z is CR⁵;
A, B and D are each independently selected from O, CR⁶, S and NR⁶;
R⁴ and R⁶ are each independently selected from hydrogen and C₁₋₄ alkyl, where said alkyl is unsubstituted or is substituted with halogen, hydroxyl or C₁₋₄ alkoxy; and
R⁵ is independently selected from hydrogen, halogen, hydroxyl, C₁₋₄alkyl optionally substituted with one or more substituents selected from halogen and hydroxyl, C₁₋₄ alkoxy, cyano and N(R⁴)₂;
and pharmaceutically acceptable salts and individual enantiomers and stereoisomers thereof.

3. A compound of claim 2 having Formula Ib: 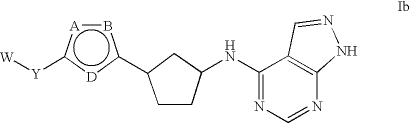
wherein:
W is aryl or heteroaryl, unsubstituted or substituted with 1-5 substituents independently selected from halogen, C₁₋₆ alkyl optionally substituted with one or more of halogen, hydroxyl and C₁₋₄ alkoxy, C₁₋₄ alkoxy, C₃₋₆ cycloalkyl, cyano, and N(R⁴)₂;
Y is absent or is selected from C₁₋₃ alkyl unsubstituted or substituted with one or more substituents selected from halogen, hydroxyl and C₁₋₄ alkoxy, cyclopropyl and C(O);
A, B and D are each independently selected from O, CR⁶, S, and NR⁶;
R⁴ and R⁶ are each independently hydrogen, C₁₋₄ alkyl unsubstituted or substituted with one or more substituents selected from halogen and hydroxyl;
and pharmaceutically acceptable salts and individual enantiomers and stereoisomers thereof.

4. A compound of claim 2 having Formula Ic: 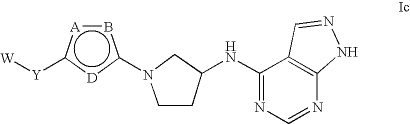
wherein:
W is aryl or heteroaryl, unsubstituted or substituted with 1-5 substituents independently selected from, halogen, C₁₋₆ alkyl unsubstituted or substituted with one or more of halogen, hydroxyl or C₁₋₄ alkoxy, C₁₋₄ alkoxy, C₃₋₆ cycloalkyl, cyano and N(R⁴)₂;
Y is absent or is selected from C₁₋₃ alkyl unsubstituted or substituted with one or more substituent selected from halogen, hydroxyl and C₁₋₄ alkoxy, cyclopropyl and C(O);
A, B and D are each independently selected from O, CR⁶, S, and NR⁶, and
R⁴ and R⁶ are each independently hydrogen, C₁₋₄ alkyl unsubstituted or substituted with one or more substituent selected from halogen and hydroxyl;
and pharmaceutically acceptable salts and individual enantiomers and stereoisomers thereof.

5. A compound of claim 1 selected from: 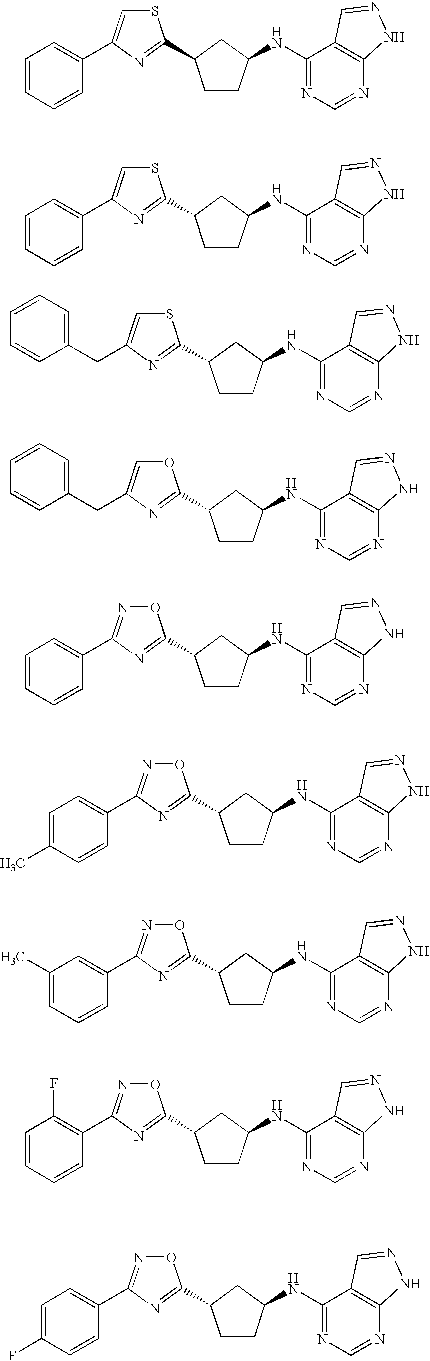 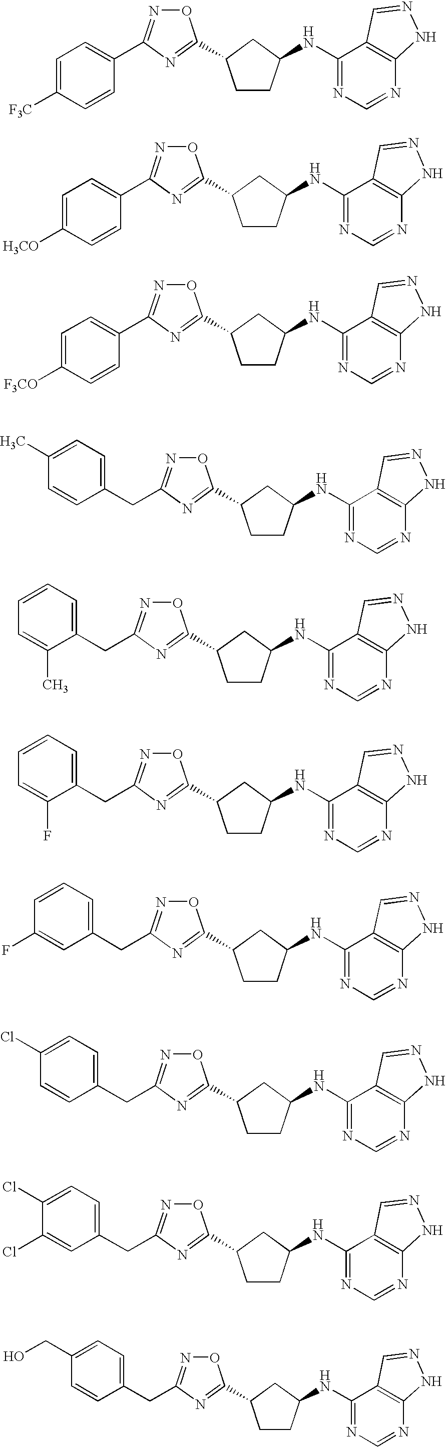 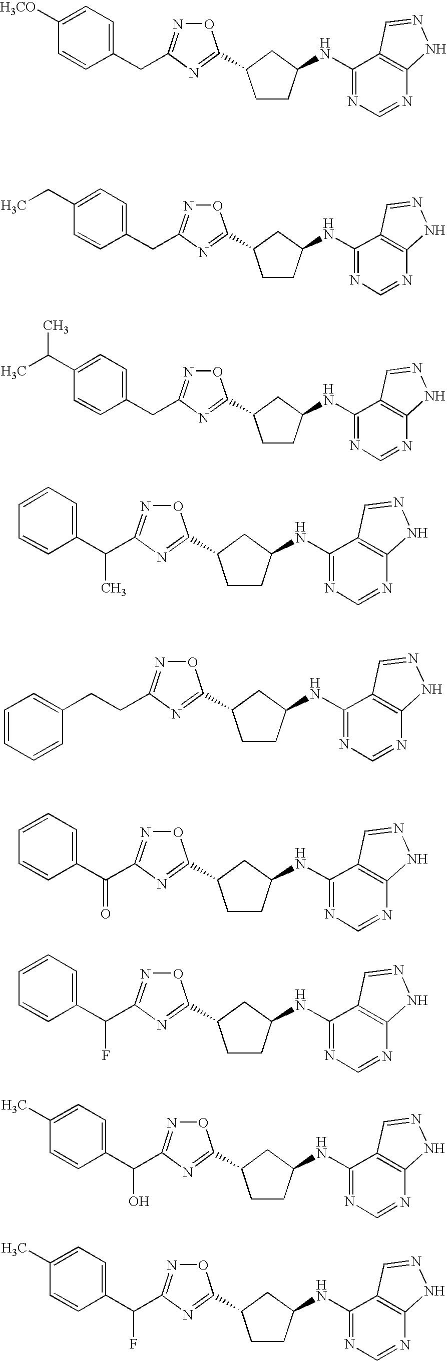 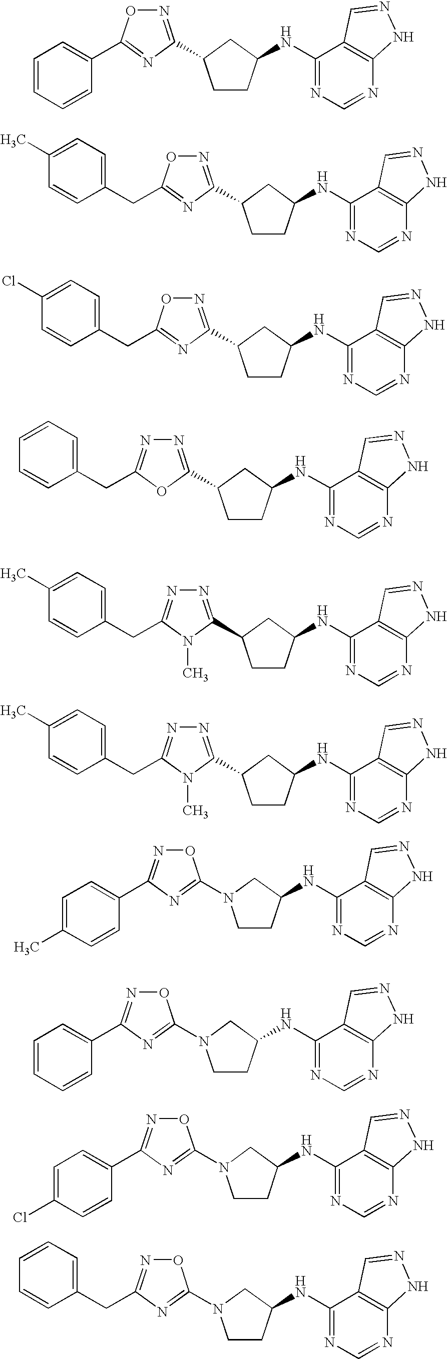 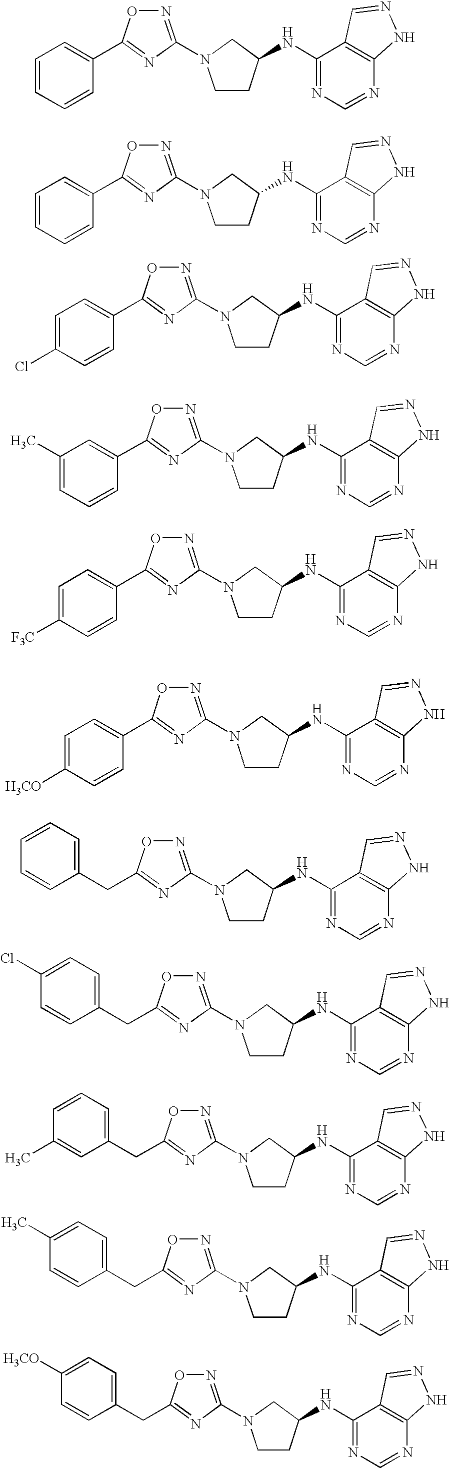 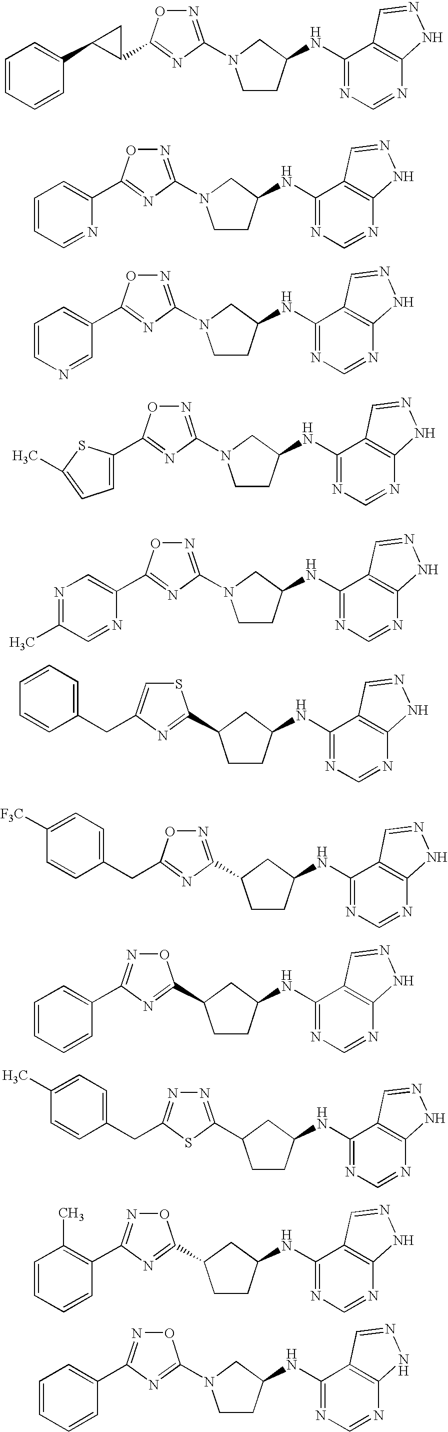 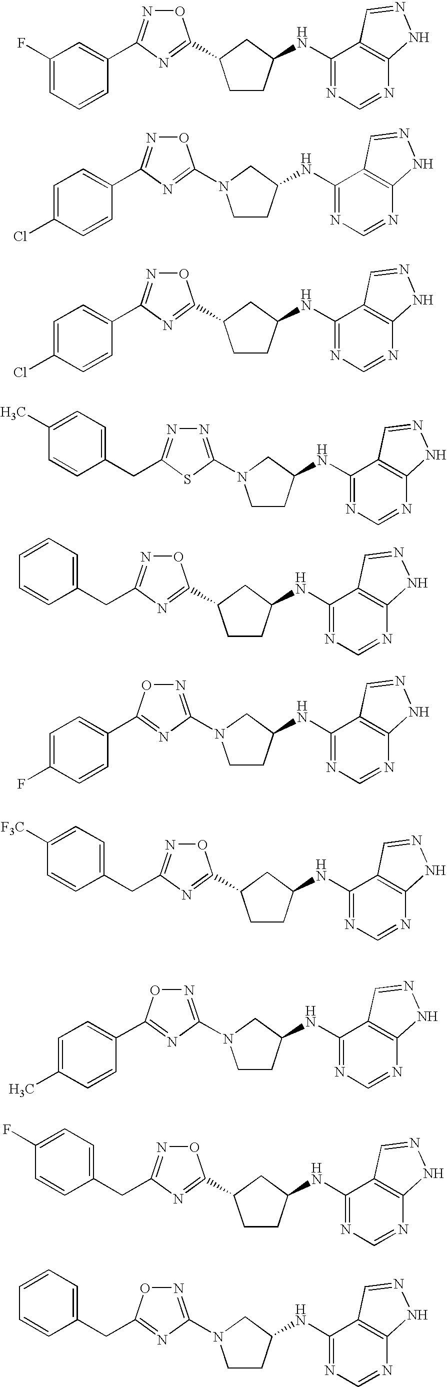 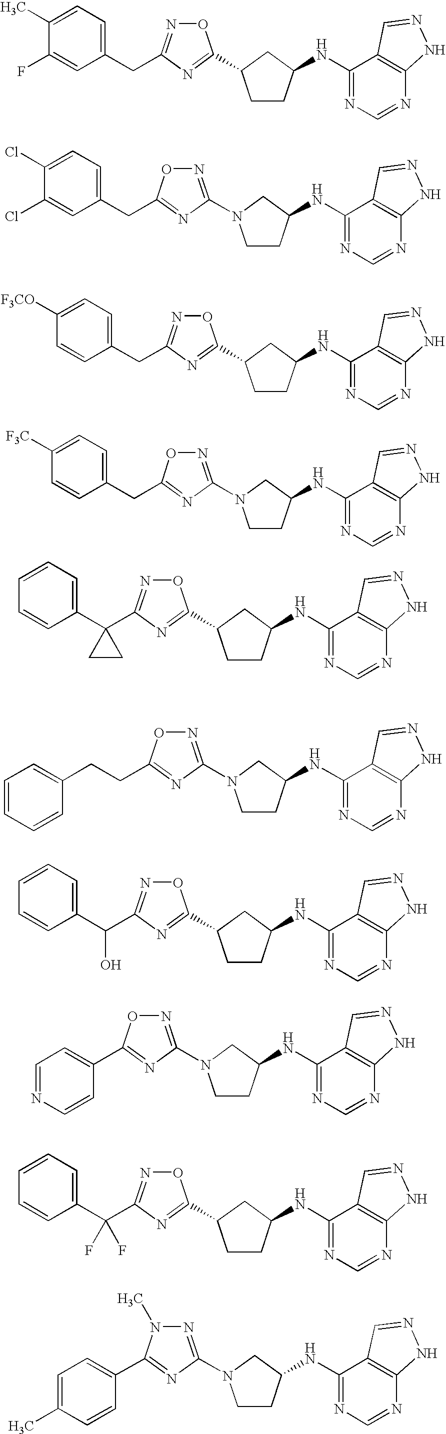 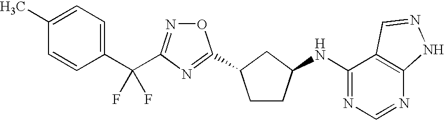
and pharmaceutically acceptable salts and individual enantiomers and stereoisomers thereof.

6. A pharmaceutical composition comprising an inert carrier and a therapeutically effective amount of a compound according to claim 1.

7. The pharmaceutical composition according to claim 6, further comprising a second therapeutic agent selected from the group consisting of: non-steroidal anti-inflammatory agents; COX-2 inhibitors; bradykinin B1 receptor antagonists; sodium channel blockers and antagonists; nitric oxide synthase (NOS) inhibitors; glycine site antagonists; potassium channel openers; AMPA/kainate receptor antagonists; calcium channel antagonists; GABA-A receptor modulators (e.g., a GABA-A receptor agonist); matrix metalloprotease (MMP) inhibitors; thrombolytic agents; opioids such as morphine; neutrophil inhibitory factor (NIF); L-Dopa; carbidopa; levodopa/carbidopa; dopamine agonists such as bromocriptine, pergolide, pramipexole, ropinirole; anticholinergics; amantadine; carbidopa; catechol O-methyltransferase (“COMT”) inhibitors such as entacapone and tolcapone; Monoamine oxidase B (“MAO-B”) inhibitors; opiate agonists or antagonists; 5HT receptor agonists or antagonists; NMDA receptor agonists or antagonists; NK1 antagonists; selective serotonin reuptake inhibitors (“SSRI”) and/or selective serotonin and norepinephrine reuptake inhibitors (“SSNRI”); tricyclic antidepressant drugs; norepinephrine modulators; lithium; valproate; and (33) neurontin (gabapentin).

8. A method for the treatment or prophylaxis of diseases and disorders mediated through the NR2B receptor which comprises administering to a subject in need thereof a therapeutically effective amount of a compound of claim 1.

9. The method of claim 8 wherein said diseases and disorders mediated through the NR2B receptor is pain.

10. The method according to claim 9, wherein said pain includes neuropathic pain, central pain syndromes, postsurgical pain syndromes, pain resulting from osteoarthritis, repetitive motion pain, dental pain, cancer pain, myofascial pain, perioperative pain, chronic pain, dysmennorhea, pain associated with angina, inflammatory pain, headache, migraine, cluster headache, non-vascular headache, primary hyperalgesia, secondary hyperalgesia, primary allodynia, secondary allodynia, other pain caused by central sensitization, HIV treatment-induced neuropathy, chronic pelvic pain, neuroma pain, complex regional pain syndrome, chronic arthritic pain and related neuralgias, chronic lower back pain, pain resulting from or associated with traumatic nerve injury, nerve compression or entrapment, postherpetic neuralgia, trigeminal neuralgia, diabetic neuropathy, cancer and chemotherapy.

11. The method of claim 8 wherein said diseases and disorders mediated through the NR2B receptor is selected from Parkinson's disease, depression, anxiety, schizophrenia, stroke, traumatic brain injury, Alzheimer's disease, cerebral ischemia, amyotrophic lateral sclerosis, Huntington's disease, sensorineural hearing loss, tinnitus, glaucoma, neurological damage caused by epileptic seizures, neurotoxin poisoning or impairment of glucose or oxygen to the brain, vision loss caused by neurodegeneration of the visual pathway, Restless Leg Syndrome, multi-system atrophy and dyskinesias.
